(19) United States
(12) Patent Application Publication
(10) Pub. No.: US 2004/0067506 A1
(43) Pub. Date: Apr. 8, 2004

(54) Novel root specific promoter driving the expression of a novel lrr receptor-like kinase

(76) Inventors: Ben Scheres, Ac Utrecht (NL); Renze Heidstra, Lh Utrecht (NL)

Correspondence Address: Ann R Pokalsky, Dilworth & Barrese, 333 Earle Ovington Boulevard, Uniondale, NY, 11553, US

(21) Appl. No.: 10/433,731

(22) PCT Filed: Dec. 4, 2001
(86) PCT No.: PCT/EP01/14154

(30) Foreign Application Priority Data
Dec. 4, 2000 (EP) 00870288.8

(51) Int. Cl. C12Q 1/68; C07H 21/04; C07K 14/705; C12P 21/02

(52) U.S. Cl. 435/6; 435/69.1; 435/320.1; 435/325; 530/350; 536/23.5

(57) ABSTRACT

The present invention relates to the field of plant molecular biology, more particularly to the root-specific gene expression in plants. The invention provides nucleic acids for a novel transcriptional regulatory root-specific promoter and nucleic acid and protein sequences coding for a new LRR receptor-kinase protein, further specified as a root clavata 1 homolog (RCH1). Further provided are compositions comprising nucleic acids, polypeptides, antibodies and vectors. The invention further provides for methods for modifying cell fate and/or plant development and/or plant morphology and/or plant biochemistry and/or plant physiology comprising the modification of expression in particular cells, tissues or organs of a plant of the novel LRR receptor-like kinase or comprising the expressing of a gene of interest under the control of the novel transcriptional regulatory root-specific promoter. Further are provided compounds interacting with the new polypeptides for use as herbicides or growth regulators.

### FIELD OF THE INVENTION

The present invention relates to the field of plant molecular biology, more particularly to the root-specific gene expression in plants. The isolation of a root specific operon, comprising a transcriptional regulatory promoter that contributes to root-specific gene expression and its operably linked gene encoding a novel Leucin Rich Repeat (LRR) receptor-like kinase, is disclosed. Said transcriptional regulatory promoter may be used for driving root-specific expression of at least one gene of interest in a transgenic plant and said encoded LRR receptor-like kinase gene may be used to alter the features and/or to confer a selective advantage to transgenic plants.

### BACKGROUND TO THE INVENTION

Plant Promoters

Initiation of transcription is generally understood to be the predominant controlling factor in determining expression of a gene. The transcriptional control elements, which may interact with DNA binding proteins, are generally embedded in the sequence 5′-flanking or upstream of the transcribed gene. These DNA sequence elements promote the formation of transcriptional regulatory complexes that either activate or repress the expression of the gene 3′ downstream of the promoter.

The sequences of the promoter vary in length and base pair composition from gene to gene. Within the sequence of the promoter, many regulatory sequence elements can be embedded that control the promoter activity. These specific elements, called DNA-boxes, contribute to the promoter a defined feature or a defined activation pattern. Many of these boxes are recognition sites to which regulatory transcription factors can bind and are part of a tight control mechanism of the promoter. Numerous plant promoter DNA boxes have been described, such as tissue-specific boxes (Chaubet et al. 1996), pyrimidin boxes (Gubler & Jacobsen 1992), which Influence the level of expression or G-boxes (Dolferus et al. 1994) which reduce promoter activity by cold or dehydratation.

Constitutive promoters are promoters that drive the expression of genes in a continuous manner, whereas ubiquitous promoters drive the expression of the gene throughout the entire plant.

Expression of heterologous DNA sequences in a host is dependent upon the presence of an operably linked promoter that is also present in that host, whereby the choice of promoter will determine when, where or how strong the heterologous DNA sequence will be expressed in the host organism. Frequently those skilled in the art desire to limit the expression of the transfected DNA sequence to a certain time-period (e.g. to a particular phase of plant development), to a defined tissue of the host (e.g. leaves), or to a certain expression level. Controlling the expression of the heterologous genes in transgenic plants is considered to provide several advantages to the plant over ubiquitous and constitutive expression. There is a broad need in many industrial applications for transcriptional control elements capable of driving tissue specific gene-expression in plants. It is therefore important that such control elements are continuously provided and that new control elements with their specific transcriptional features are isolated and characterized.

Promoter Regulation

Promoters from both monocots and dicots that have a root root-preferred expression pattern have already been described (Fowler, Bernhardt, & Tierney 1999; Hwang & Goodman 1995; Lagarde et al. 1996; Okada et al. 2000; Okumura et al. 1994; Pysh et al. 1999; Schnelder et al. 1998; Vidmar et al. 2000; Wanapu & Shinmyo 1996; Winicov 2000) WO9007001(Baszczynski et al. 2000; Borg et al. 1997; Masuda, Sakuta, & Satoh 1999; Webb et al. 2000) (de Pater & Schilperoort 1992; Ely, Evans, & Schuch 2000; Goddemeier, Wulff, & Feix 1998; Kikuchi et al. 1999; Suzuki, Fowler, & Tierney 1993; Uribe et al. 1998; Woo & Hawes 1997; Xu et al. 1995) (Yamamoto et al. 1991) (Giritch et al. 1997; Keller & Lamb 1989; Lauter 1996; Olson, Oetiker, & Yang 1995; van der Zaal et al. 1991). Many of them are root-preferred rather then root-specific and are expressed at low levels in tissues other than the root. These promoters are not suitable for industrial applications where it us of the utmost importance that the heterologous product is expressed in the root only. As an example, ectopic expression of cell cycle control elements to obtain growth arrest in some parts of the root (e.g. lateral roots), should be strictly regulated to that tissue in order not to interfere with the overall growth of the plant. In contrast, other promoters are strictly and exclusively confined to root-specific expression and can therefore be valuable for some applications demanding tight control of the expression. In this group of root-specific promoter subgroups can be distinguished based on the root-tissue they are expressed in. The HRGPnt3 gene of tobacco for instance is only expressed in the pericycle and the endodermis of the root, more specifically in the discrete subset of cells, involved in the initiation of the lateral roots (Keller et al. 1989). This makes the promoter of HRGPnt3 highly specific. Another example is the XSP30 sequence, which is only activated in the xylem of the root.

For persons skilled in the art it is of importance to have at their disposal a wide variety of root-specific promoters with different root tissue-specificity from which they can choose to confer a specific feature to the transgenic plant.

Many of these root-preferred promoters are not only limited to tissue specific expression, but are also subjected to a tight control mechanism deciding when the promoter should be active. This decision depends on the function and the effects of the gene product that is switched on/off by the related promoter. Some gene products are switched on/off at a certain developmental stage of the plant, at a certain moment of the cell cycle, in stress situations originating from weather factors, upon injury caused by pathogens or upon a change in the environmental resources. A typical example is the ARSK1 gene of Arabidopsis thaliana, that is activated when the roots are exposed to air during growth (dehydration) or by treatment of roots with abeisic acid or salt (Hwang et al. 1995).

However, for many industrial and agronomic applications, where the aim is to confer a special feature at any time of the developmental, environmental or physical state of the plant, a constitutive promoter will be needed. For example when the gene product of the transgene is to be isolated and purified from the plant for commercial purposes.

It is important for the persons skilled in the art to have at their disposal a wide variety of promoters with different time-control features from which they can chose to confer a specific feature to the transgenic plant.

Plant Root Development

Root development is an essential determinant of plant growth and crop yield since the root is the main channel to extract nutrients from the environment. Any changes in the root that contribute to more functional organ tissue will result in higher crop yield. Also any regulatory element that is involved in the root development and whose control is subjected to developmental events can contribute better yield properties to a transgenic plant.

Cells of the shoot and root meristems are mitotically active and they extend the undifferentiated tissue pattern of the embryo as a pool of cells from which cells can differentiate into several tissues of the adult plant. This process continues throughout the life span of the plant. It is clear that the root meristem plays a pivotal role in the formation of strong roots and therefor also in affecting the growth and crop yield.

In root meristem, the emerging picture is that the integrity of the root meristem is kept by balancing cell proliferation and cell differentiation, and that differentiation-inhibiting signals originating from the quiescent center are involved (van den Berg et al. 1997). Inhibition of differentiation only occurs in cells that contact the quiescent center, which reveals that short-range or contact-dependent signals are involved in this process. However which compounds play a role in this process and whether positional cues guiding root cell fate are targeted via plasmodesmata or via the cell wall remains unknown (van den Berg et al. 1997).

In the shoot meristem more progress has been made in characterizing molecular components involved in meristem maintenance. Clavata (clv) mutants accumulate massive pools of undifferentiated cells in the central zone of shoot and floral meristem. The CLAVATA genes show a shoot and floral meristem specific expressionpattern. Root meristems of clv-mutants are unaffected (Clark, Running, & Meyerowitz 1993). The CLAVATA loci (CLV1, CLV2 and CLV3 appear to promote the transition toward differentiation of cells at the shoot and floral meristems, and/or to restrict the proliferation of cells at the center of these meristems (Clark et al. 1993). CLV1 acts with CLV3 and WUSCHEL (WUS) and SHOOT MERISTEMLESS (STM) to maintain the integrity of the central zone which acts as a reservoir of stem cells. A model for stem-cell maintenance in the root meristem and in the shoot meristem is depicted in FIG. 1.

CLV1 Homologs and CLV1-Redundancy

The CLAVATA1 gene resides on the Arabidopsis thaliana chomosome 1. Clv1 mRNA is found in the L3 tunica cell layer of the central zone and encodes a protein with leucine-rich repeats (LRR, of a type found in a number of proteins, notably the products of several plant-pathogen resistance genes), a putative serin/threonin kinase domain and a predicted extracellular receptor domain. Expression of the clv1 gen is initiated in the heart stage of the embryo, when cotyledonary primordia are apparent, and is independent of the STM (SHOOT MERISTEMLESS) activity (Long & Barton 1998). CLAVATA1 is a leucine-rich receptor-like kinase. The Arabidopsis thaliana genome project has so far identified over 50 LRR transmembrane receptor serine/threonine kinases. It can be estimated that the total Arabidopsis thaliana genome contains in the order of 100 family members. Other members of the CLAVATA family are ERECTA (ER) (Torii et al. 1996), BRASSINOLIDE INSENSITVE (BRI) (Li & Chory 1997) and HAESA (HAE) (Jinn, Stone, & Walker 2000), genes which have a function in plant development. Analogues of the Arabidopsis thaliana CLAVATA genes were also found in soybean (Glycine max). They were isolated from the wild type and the fasciating mutant of soybean and were designated GmCLVA1 and GmCLV1B (Yamamoto, Karakaya, & Knap 2000).

It is the aim of the present invention to isolate novel promoters and genes which are useful for altering features of transgenic plants and thus confer selective advantage to transgenic plants.

All the aims of the present invention have been met by the formulation of the following preferred embodiments of the current invention.

### SUMMARY OF THE INVENTION

In the present invention a DNA operon was isolated from Arabidopsis thaliana, which comprises a novel root-specific promoter, driving a gene encoding a Leucine Rich Repeat (LRR) receptor-like kinase. Unexpectedly, the inventors discovered that the new operon was specifically expressed in roots and more surprisingly in the meristem of the main and lateral roots, in the main and lateral root vascular tissue and in the lateral root primordia.

The surprisingly specific expression pattern, as well as the very strong expression level, makes the newly found promoter a very attractive and selective tool to drive root-specific expression of any gene of interest. The ability to target meristem cells of the root makes this promoter clearly distinct from the root-specific promoters presently known in the art. Another surprising aspect of the invention is the identification of the new LRR receptor-like kinase gene, which is the first gene identified in roots showing homology with a clavata-gene. This gene is specifically expressed in roots in a specific developmental stage of the root cells (namely in the meristematic phase) and can therefore contribute agronomic interesting features to a transgenic plant when transfected herein. Therefore the root-specific promoter is further denominated as ROOT CLAVATA HOMOLOG 1 promoter (RCH1 prom) and the LRR receptor-like kinase as RCH1.

Accordingly, the invention embodies an isolated DNA sequence with nucleotide sequence as given in SEQ ID NO 1 (FIG. 5) comprising a novel root specific regulatory promoter sequence with nucleotide sequence as given in SEQ ID NO 2 (FIG. 6) or SEQ ID NO 18 (FIG. 13), and an isolated DNA sequence with nucleotide sequence as given in SEQ ID NO 3 (FIG. 8), encoding a novel ROOT CLAVATA HOMOLOG 1 gene, with amino acid sequence given in SEQ ID NO 4 or SEQ ID NO 19 (FIG. 9). Furthermore the invention embodies a method for modifying cell fate and/or plant development and/or plant morphology and/or biochemistry and/or physiology comprising the modification of expression in particular cells of the root of a particular transgene operably linked to the RCH1 promoter sequence of the present invention. Provided in the current invention are methods to effect expression of any gene of interest in particular cells of the plant root. The present invention also relates to a method to effect expression of a ROOT CLAVATA HOMOLOG 1 protein or a homologue or a derivative thereof in a plant cell, tissue or organ. The present invention clearly extends to any plant produced by the inventive methods described herein.

### DETAILED DESCRIPTION OF THE INVENTION

One of the technical problems underlying the present invention is to provide regulatory elements that are active in the root only and that show no expression in other tissues. Additional to the highly selective and tissue-specific regulation of a promoter, other features such as very strong activity, continuous activity, and termination of activity at a certain developmental stage of the cells, would contribute an unique combination of regulatory elements to specifically regulate the expression of a gene of interest in the roots of a transgenic plant, thereby providing a means for modifying cell fate and/or plant development and/or plant morphology and/or biochemistry and/or physiology. Also the isolation of root-specific DNA sequences encoding proteins that could play a role in root development is envisaged for use in modification of cell fate and/or plant development and/or plant morphology and/or plant biochemistry and/or plant physiology.

In a first embodiment, the present invention relates to an isolated nucleic acid comprising a transcriptional regulatory root promoter sequence and/or a nucleic acid sequence encoding a novel LRR receptor-like kinase (protein) or an immunologically active and/or functional fragment of such a regulary element or protein (kinase) selected from the group consisting of:

(a) nucleic acid comprising at least part of the DNA sequence as given in any of SEQ ID NOs 1, 2, 3 or 18, or the complement thereof,

(b) nucleic acid comprising the RNA sequences corresponding to at least part of any of SEQ ID NOs 1, 2, 3 or 18, or the complement thereof,

(c) nucleic acid specifically hybridizing, preferably under stringent conditions, with the nucleotide sequence as defined in (a) or (b),

(d) nucleic acid encoding a protein comprising the amino acid sequence as given in SEQ ID NO 4, SEQ ID NO 5 or SEQ ID NO 19,

(e) nucleic acid which is degenerated as a result of the genetic code to a nucleotide sequence encoding a protein as given in SEQ ID NO 4, SEQ ID NO 19 or to a nucleotide sequence as defined in (a) to (d),

(f) nucleic acid which is diverging due to the differences in codon usage between the organisms to a nucleotide sequence encoding a protein as given in SEQ ID NO 4, SEQ ID NO 19 or to a nucleic acid sequence as defined in (a) to (d),

(g) nucleic acid encoding a protein as given in SEQ ID NO 4, SEQ ID NO 19 or as defined in (a) to (d), which is diverging due to the differences between alleles encoding a protein

(h) nucleic acid encoding a fragment of a protein encoded by a DNA sequence as given in SEQ ID NO 1 or SEQ ID NO 3 or encoding a fragment of a protein encoded by a nucleic acid as defined in any one of (a) to (g), wherein said fragment comprises the sequence as represented in SEQ ID NO 5, and,

(k) nucleic acid encoding a protein as defined in SEQ ID NO 4, SEQ ID NO 19 or nucleic acid as defined in any one of (a) to (h), said nucleic acid interrupted by intervening DNA sequences.

The sequence as depicted in SEQ ID NO 1 is part of a sequence deposited under the Genbank Accession number ABO17061, which represents the entire Arabidopsis thaliana chromosome 5. The nucleic acid of the invention is not the nucleic acid with the sequence as deposited under the Genbank Accession number ABO17061 (FIG. 14). Furthermore, in a preferred embodiment, the present invention relates to an isolated nucleic acid comprising a novel root-specific promoter sequence, termed RCH1 prom, isolated from Arabidopsis thaliana and consisting of the nucleic acid as represented in SEQ ID NO 2 or SEQ ID NO 18, or a functional part of said sequence, which is able to regulate gene expression in a root specific manner as SEQ ID NO 2 or SEQ ID NO 18 itself. More specifically, said isolated DNA sequence provides a novel type of a root-specific promoter, which differs from other known root-specific promoters in the fact that it is unexpectedly active in only distinct cells of the root, namely the meristem of the main and the lateral root, as well as in the vascular tissue of the main and lateral root and in the lateral root primordia.

The isolation of the root specific promoter RCH1 of the present invention is described in Example 1.

Another aspect of the invention is the isolation and sequencing of a root-specific Leucin Rich Repeat (LRR) receptor-like kinase gene from root tissue of Arabidopsis thaliana and is also described in Example 1. The nucleotide sequence is listed in the current specification as SEQ ID NO 3 and illustrated in FIG. 8. This sequence is present as part of the sequence deposited under the GenBank Accession number ABO17061, which represents the entire Arabidopsis thaliana chromosome 5.

The novelty of the RCH1 promoter sequence relative to known root-specific promoters is substantiated by its unique expression pattern in plant roots. The analysis of the RCH1 expression patterns was done using a RCH1 prom-GUS hybrid operon that was transformed to Arabidopsis thaliana as outlined in Example 2. Analogous experiments with the RCH1prom-GUS hybrid operon were performed to establish the expression pattern of RCH1 in embryo's (Example 8 and FIG. 15).

The RCH1 promoter is active in the meristem of the main and the lateral roots, as well as in the vascular tissue of the main and the lateral root and in the lateral root primordia. The promoter of the present invention is the first known promoter that regulates a CLAVATA HOMOLOG gene in roots, and is clearly distinct from other described CLAVATA-operons which are all active in the shoot and floral meristem. The present data thus clearly distinguish RCH1prom from other plant root-specific promoters, which are not expressed in the meristem of the main and lateral root, in the vascular tissue of the main and lateral root and in the lateral root primordia.

From the results obtained by the RCH1::GFP expression studies in root it was even more clear that the RCH1 promoter is strongly active in the endodermis, cortex, epidermis, lateral root cap, in short in the “division zone”. The RCH1 promoter is also active but in a lower manner, in the quiescent center and in the vascular tissue.

A further characteristic of the RCH1 promoter is its very high expression level. Based on the incubation time of the RCH1prom-GUS hybrid operon transformed plant with the GUS substrate (1 hour) and the very high output signal in the root of that plant (Example 2, FIG. 7D), it is clear for persons skilled in the art that the RCH1 promoter is very strong.

A further characteristic of the present invention is that the activity of the RCH1promoter in the meristem cells stops when these cells start to differentiate and to elongate to become cells of specified root tissue other than root vascular tissue. Surprisingly the promoter is activated again in cells of the pericycle that form lateral root primordia. This expression is extended in the meristem of the lateral root, but again, the cells that differentiate into lateral root tissue other than vascular tissue do not retain RCH1 promoter activity.

Functional parts of the promoter of the invention will have at least one of these characteristics.

According to a further embodiment, the present invention relates to an isolated nucleic acid comprising a transcriptional regulatory root promoter comprising at least part of a sequence as given in SEQ ID NO 1, 2 or 18.

According to another embodiment, the present invention relates to an isolated nucleic acid as represented in SEQ ID NO 2 or 18 wherein said transcriptional regulatory root promoter is operably linked to said nucleic acid sequence encoding a novel LRR receptor-like kinase or to an immunologically active and/or functional fragment thereof.

According to the invention, distinctive fragments of the newly identified promoter, which contribute specific features to said promoter, can be fused to another nucleic acid sequence (e.g. a promoter) in order to alter the sequential and/or regulatory features of the latter.

The invention thus relates to an isolated nucleic acid having transcriptional regulatory root specific, root-meristem specific or root-vascular-tissue specific promoter activity comprising at least part of the DNA sequence as given in any of SEQ ID 1, 2 or 18 and a second transcriptional regulatory sequence.

In one embodiment, said second trancriptional regulatory sequence is for instance an inducible box or sequence, which is placed within the RCH1 promoter, wherein the function of the RCH1 promoter is not hampered but instead can be regulated, for instance by induction. In the latter embodiment, the RCH1 promoter is not used to confer root-specificity to other promoters, but a second sequence is used to confer an additional regulatory feature to the RCH1 promoter.

In another embodiment said second transcriptional regulatory sequence is a promoter sequence not normally exhibiting root-specificity, thereby resulting in a hybrid promoter with root-specificity.

Accordingly, another embodiment of the present invention is to confer root-specificity, root-meristem-specificity, and root-vascular-tissue-specificity and/or root abundant gene expression to other promoter sequences. This can be achieved by fusing elements of the here-disclosed RCH1 promoter to the promoter sequence of interest preferably the widely used ubiquitin promoter. Such modifications can be achieved by routine experimentation by those skilled in the art.

It should be further understood that the invention preferably relates to any of the isolated nucleic acids as described above which is DNA, cDNA, genomic DNA, synthetic DNA or RNA wherein T is replaced by U.

The invention also relates to a nucleic acid molecule of at least 15 nucleotides in length hybridizing specifically with any of the nucleic acids of the invention. The invention also relates to a nucleic acid molecule of at least 15 nucleotides in length specifically amplifying a nucleic acid of the invention.

Further, the invention relates to a vector comprising a nucleic acid of the invention. Said vector may be an expression vector wherein the (said) nucleic acid sequence encoding a novel LRR receptor-like kinase or an immunologically active and/or functional fragment thereof, is operably linked to one or more control sequences allowing the expression in prokaryotic and/or eukaryotic host cells.

The invention also relates to a vector comprising at least part of a nucleic acid according to the invention and wherein a transcriptional regulatory root promoter of the invention or functional part(s) thereof, is operably linked to one or more genes of interest.

The invention also relates to a host cell containing a nucleic acid molecule of the invention or a vector of the invention wherein said nucleic acid or vector has been introduced by transfomation, transfection or infection. Preferred host cells that are part of the invention are bacterial, insect, fungal, plant or animal cells.

Therefore, in a preferred embodiment, the invention relates to an isolated nucleic acid as represented in SEQ ID NO 3 encoding a LRR receptor-like kinase characterized by an amino acid sequence as given in SEQ ID NO 4 or 19. More specifically, said isolated nucleic acid encodes a plant ROOT CLAVATA HOMOLOG 1 (RCH1) of a novel type which is unexpectedly expressed in roots. Said novel plant RCH1 shows significant homology with CLAVATA 1, a protein involved in the maintenance of floral and shoot meristem cells.

An initial comparative amino acid sequence homology search using the BLASTP 2.0.8 software (Altschul et al. 1997) revealed that most significant alignments of RCH1 protein are produced with receptor-like protein kinases, more specifically with leucine rich receptor-like kinases such as CLAVATA1 (CLV1). Furthermore, the most homologous protein with a known function of this list, is the CLAVATA 1 receptor-like kinase from Arabidopsis thaliana. Therefore the novel gene was denominated a ROOT CLAVATA HOMOLOG 1 gene (RCH1).

An amino acid sequence alignment of the RCH1 protein and CLAVATA 1 from A. thaliana is shown in FIG. 10. Calculation of the percentage of identical amino acids residues between RCH1 and CLV1 teaches that RCH1 is 39% identical to CLV1. Additional to 39% of identical amino acids, there is 49% of the amino acids that belong to the same class of amino acids and therefore have the same physical and chemical properties.

The classification of RCH1 as a novel type of CLAVATA is documented further in FIGS. 11 and 12.

The conclusions we can draw from FIG. 11 is that based on the sequence, RCH1 is closer related to CLV1 compared to other known CLV1 homologues, such as ERECTA, BRASSINOLIDE INSENSITIVE and HAESA.

The fact that the novel LRR receptor like protein described in the present invention is rather a root clavata 1 homologue then another kind of LRR receptor-like kinase (e.g. resistance genes) is illustrated in FIG. 12 in which the evolutionary relationship between clavata homologues and resistance genes is shown. The resistance genes (RPS5, RPS2, RPP8, RPM1, RPS4, N, RPP5) are described in “Meyers BC, Dickerman A W, Michelmore R W, Sivaramakrishnan S, Sobral B W, Young N D. Plant disease resistance genes encode members of an ancient and diverse protein family within the nucleotide-binding super family. Plant J. 1999 November; 20(3):317-32”.

The present invention relates to a ROOT CLAVATA HOMOLOG 1 gene that differs from other known CLAVATA-like genes, in the fact that it is active in the root meristem, whereas the other known CLAVATA genes and CLAVATA homologs (like ERECTA, BRASSINOLIDE INSENSITIVE and HAESA) are only active in the shoot and floral meristem.

According to another embodiment, the invention also relates to nucleic acids encoding a functional plant LRR receptor-like kinase comprising one or more protein regions, distinguishing said plant LRR receptor-like kinase from those LRR receptor-like kinases known in the art.

The inventors found that the predicted amino acid sequence (BAB10317.1, receptor protein kinase-like protein) corresponding to the database entry with accession number ABO17061 (A. thalinana chromosome 5), was missing a part of the amino acid sequence (namely, 25 amino acids as represented in SEQ ID NO. 5 of the present patent application). This extra stretch of 25 amino acids was identified by the cloning and the sequencing of the corresponding cDNA of the present invention. Therefore, the predicted prior art sequence is believed not to represent a functional enzyme.

The invention thus relates to an isolated nucleic acid selected from the group consisting of:

(a) a nucleic acid consisting of at least part, preferably at least a functional part, of the DNA sequence as given in SEQ ID NO 1 or 3, or the complement thereof,

(b) a nucleic acid encoding a protein as given in SEQ ID NO 4 or 19 or encoding a fragment of said protein, wherein said fragment comprises the sequence as represented in SEQ ID NO 5, and

(c) a nucleic acid encoding a protein with an amino acid sequence which is at least 60%, preferably at least 65%, 70%, 75%, 80%, 85%, more preferably at least 90% or 95% identical to the protein as given in SEQ ID NO 4 or 9, wherein said amino acid sequence comprises the sequence as represented in SEQ ID NO 5, characterised in that said nucleic acid encodes a novel LRR receptor-like kinase protein or an immunologically active and/or functional fragment of such a protein, and further provided that said nucleic acid is not one of the nucleic acids as deposited under the GenBank Accession numbers ABO17061 or AQ966419.

The present invention also relates to an isolated LRR receptor-like kinase (protein) comprising one of the polypeptides selected from the group consisting of:

(a) a polypeptide as given in SEQ ID NO 4 or SEQ ID NO 19,

(b) a polypeptide comprising the amino acid sequence as given in SEQ ID NO 5, and,

(c) a polypeptide comprising the sequence represented in SEQ ID NO 5 and encoded by a nucleic acid as given in SEQ ID NO 1 or 3,

or a homologue or a derivative of said protein, or a fragment or an immunologically active and/or functional fragment thereof, provided that said homologue is not the amino acid sequence as described under the GenBank Accession number BAB10317.1.

With the expression “homologue” when referring to homologues of the LRR receptor-like kinase (protein), a polypeptide is meant which is at least 65% identical to the amino acid sequence of RCH1 as represented in SEQ ID NO 4 or 19.

More preferred, the invention relates to a protein consisting of an amino acid sequence as given in SEQ ID NO 4 or SEQ ID NO 19. More specifically, the present invention relates to a root CLAVATA homologue or a functional homologue thereof. A preferred fragment of said protein is represented in SEQ ID NO 5.

A further embodiment of the invention comprises homologues, derivatives, immunologically active and/or functional fragments of the LRR receptor-like kinase (protein) according to the invention, and proteins comprising said homologues, derivatives, immunologically active and/or functional fragments of said LRR receptor like kinase (protein).

As such, the present invention also relates to an isolated polypeptide comprising the sequence represented in SEQ ID NO 5 encodable by a nucleic acid molecule of the invention as defined above, or a homologue or a derivative thereof, or a fragment or an immunologically active and/or functional fragment thereof.

In a more preferred embodiment, the invention relates to a polypeptide, encodable by a nucleic acid molecule of the invention and which has an amino acid sequence as given in SEQ ID NO 4 or SEQ ID NO 19 or comprising an amino acid sequence as given in SEQ ID NO 5, or a homologue or a derivative thereof, or an immunologically active and/or functional fragment thereof.

Any of said proteins can be produced in a biological system, e.g. a cell culture. Alternatively any of said proteins is chemically manufactured e.g. by solid phase peptide synthesis. Said proteins or fragments thereof can be part of a fusion protein as is the case in e.g. a two-hybrid assay which enables e.g. the identification of proteins interacting with the LRR receptor-like kinase according to the present invention.

Therefore, according to another embodiment, the invention also relates to a method for producing a polypeptide or a protein of the invention comprising culturing a host cell further specified above under conditions allowing the expression of the polypeptide and recovering the produced polypeptide from the culture.

The proteins or fragments thereof obtained by a method of the invention are furthermore useful e.g. to modulate the interaction between a LRR receptor-like kinase according to the invention and a ligand of the receptor and/or other identified interacting protein partners. Chemically synthesized peptides are particularly useful e.g. as a source of antigens for the production of antisera and/or antibodies.

The current invention thus furthermore encompasses antisera and/or antibodies specifically recognizing the LRR receptor-like kinase according to the invention or immunologically active parts or epitopes thereof. Said antisera and/or antibodies are useful in many areas related to the invention including: (i) identification in any organism, preferably plants, of other LRR receptor-like kinase and their genes according to the invention; (ii) quantification of synthesis in organisms and/or recombinant organisms of the LRR receptor-like kinase according to the invention; (iii) purification of the LRR receptor-like kinase according to the invention; (iv) immunoprecipitation of the LRR receptor-like kinase according to the invention e.g. as a way to identify other protein partners complexing with said LRR receptor-like kinase; (v) immunolocalization of the LRR receptor-like kinase according to the invention which is expressed in an organism or a recombinant organism.

The invention also relates to a method for the production of transgenic plants, plant cells or plant tissues comprising the introduction of a nucleic acid molecule of the invention in an expressible format or a vector as described earlier in said plant, plant cell or plant tissue.

The invention also relates to a method as described above further comprising regenerating a plant from said plant cell.

In a further embodiment, the invention also relates to a transgenic plant cell comprising a nucleic acid sequence of the invention which is operably linked to regulatory elements allowing transcription and/or expression of said nucleic acid in plant cells or obtainable by one of the methods described above. Preferably, in said transgenic plant cell one of the nucleic acids of the invention is stably integrated into the genome of said plant cell. The invention further relates to a transgenic plant or plant tissue comprising said plant cells and to a harvestable part of said plant or tissue. Said harvestable part of said plant or tissue is preferably selected from the group consisting of seeds, leaves, fruits, stem cultures, rhizomes, tubers and bulbs.

The invention further extends to the progeny derived from any of the plants or plant parts described above.

The present invention is applicable to any plant, in particular a monocotyledonous plants and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Diheteropogon amplectens, Dioclea spp, Dolichos spp., Dorycnium rectum, Echinochloa pyramidalis, Ehrartia spp., Eleusine coracana, Eragrestis spp., Erythrina spp., Eucalyptus spp., Euclea schimperi, Eulalia villosa, Fagopyrum spp., Feiloa sellowiana, Fragaria spp., Flemingia spp, Freycinetia banksii, Geranium thunbergii, Ginkgo biloba, Glycine javanica, Gliricidia spp, Gossypium hirsutum, Grevillea spp., Guibourtia coleosperma, Hedysarum spp., Hemarthia altissima, Heteropogon contortus, Hordeum vulgare, Hyparrhenia rufa, Hypericum erectum, Hyperthelia dissoluta, Indigo incamata, Iris spp., Leptarrhena pyrolifolia, Lespediza spp., Lettuca spp., Leucaena leucocephala, Loudetia simplex, Lotonus bainesii, Lotus spp., Macrotyloma axillare, Malus spp., Manihot esculenta, Medicago sativa, Metasequoia glyptostroboides, Musa sapientum, Nicotianum spp., Onobrychis spp., Omithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthda fleckii, Pogonarthria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachydum sanguineum, Sciadopitys verticillata, Sequoia sempervirens, Sequoiadendron giganteum, Sorghum bicolor, Spinacia spp., Sporobolus fimbnatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp. Vitis vinifera, Watsonia pyramidata, Zantedeschia aethiopica, Zea mays, amaranth, artichoke, asparagus, broccoli, brussel sprout, cabbage, canola, carrot, cauliflower, celery, collard greens, flax, kale, lentil, oilseed rape, okra, onion, potato, rice, soybean, straw, sugarbeet, sugar cane, sunflower, tomato, squash, and tea, amongst others, or the seeds of any plant specifically named above or a tissue, cell or organ culture of any of the above species.

Yet according to another embodiment the present invention relates to a method for conferring root-specificity and/or root-meristem-specificity and/or root-vascular-tissue-specificity and/or root-endodermis-specificity and/or root-cortex-specificity and/or root-epidermis-specificity and/or lateral-root cap-specificity and/or abundant expression in roots to other promoter sequences comprising the fusion of at least part of the DNA sequence as given in SEQ ID NO 1, 2 or 18 to a (second) transcriptional regulatory promoter sequence normally not exhibiting root-specificity.

The invention further relates to method for root-specific expression of a gene(s) of interest comprising operably linking of said gene(s) of interest to a transcriptional regulatory root-specific promoter comprising at least part of the nucleic acids as given in SEQ ID NO 1, 2 or 18 as defined above, and possibly in combination/or a (second) transcriptional regulatory promoter sequence not normally exhibiting root-specificity, as defined earlier.

The invention further relates to a method for modifying cell fate and/or plant development and/or plant morphology and/or biochemistry and/or physiology comprising the modification of expression in the meristem of the main and the lateral roots, or in the vascular tissue of the root, or in the lateral root primordia of a gene(s) of interest operably linked to a transcriptional regulatory root promoter as defined earlier.

The ability to drive tissue-specific expression of any gene of interest in plants is considered to be of great agronomic importance. This art can be used to exclude expression in eatable parts of the plant, which averts the uptake of ectopic components. Alternatively the expression of the transgene can be restricted to the relevant tissues of the plant so the total organism does not spend too much energy in superfluous production of the foreign component. This technique can also reduce potential yield loss by limiting the expression of some pernicious, yet useful agronomic genes, to specific tissues only. Said genes of interest, which can be expressed in a root-specific manner under the control of the present invention can also be involved in phytoremediation, enhanced root-growth (like cell cycle genes or cytokinin oxidase) or disease tolerance. These methods are more elaborated in detail below.

The invention described here provides a new transcriptional regulatory element which can be operably linked to a gene effecting and/or modifying root metabolites which can be involved in phytoremediation. Examples are genes involved in the production of citric and maleic acids that can be secreted by the plant and subsequently form complexes with the aluminium in the soil. Root specific expression of the aluminium-resistance gene that give rise to said metabolites can therefore allow plants to grow on acid soils, like those of south-eastern United States, Central and south America, North Africa and parts of India and China, where the aluminium is set free as an ion that poisons plant roots.

Alternatively, the promoter of the present invention also provides a tool to be used in a strategy for environmental remediation. Accordingly, a metal-resistance gene such as the TaPCS1 or the CAD1 gene, contributing cadmium tolerance, can be expressed specifically in roots to enable enhanced growth of transgenic plants on soils contaminated with cadmium. The metals can consequently be extracted from the soil and stored in the plant material, which could then be harvested and incinerated.

Therefore according to preferred embodiments, the present invention relates to a method for phytoremidiation or environmental remediation comprising the expression of a gene(s) of interest under the control of a transcriptional regulatory root promoter as defined earlier.

In conclusion the present invention provides a method for broadening the range of soils or environments in which a plant may thrive or that can be used for agriculture.

Plant root development is an essential determinant of plant growth and crop yield, so that yield could be enhanced by induced changes in expression of root-specific regulatory factors. Cyclin D for example, which promotes the cell division, can be expressed under the root-specific control of the current invention and hence contribute to increased growth rate and crop yield. Also genes that are involved in biosynthesis and perception of phytohormones like auxins, cytokinines, ethylene, abscisic acid and gibeberellins can be used to enhance root growth and development. However, the root-specific expression of these genes might be pivotal for the overall welfare of the transgenic plant.

It is known that the constitutive and ubiquitous expression of the cytokinine oxidase in Arabidopsis thaliana leads to the promotion of an enlarged root system and enhanced root biomass, but also to diminished shoot growth. To overcome this problem the cytokinin oxidase can be operably linked to the promoter of the present invention as described in example 6, which will enhance root growth without negatively affecting shoot growth. This enhanced root growth in the plant can be used for bioremidiation or phytoremediation.

Also the said gene of interest, that can be operably linked to the promoter of the present invention, can be a root transcription factor, such as Alfin. Alfin binds to promoter fragments of predominantly root-specific and salt inducible genes and its activity has major implications for crop plant yield (Winicov 2000) (Winicov & Bastola 1999)). In conclusion the present invention provides a method for enhancing plant growth and crop yield.

It is also clear for the skilled artisan that the promoter of the present invention provides a useful tool to be used for the introduction of resistance to soil-borne pathogen attack to plants, a widespread target for genetic improvement of crop plants. Certain disease-inducing microorganisms attack the belowground plant tissue and any genetic modification to contribute resistance to such organisms will require expression of the resistance-gene in the roots. To overcome this problem, the coding region that is operably linked to the promoter of the present invention preferably encodes a protein which is toxic to root-attacking organisms and more preferably the protein is an insecticidal endotoxin of Bacillus thuringiensis. Similarly to the above-mentioned pathogens, there are pests attacking plants. Certain insect species attack green leaf tissue, whereas other, for example coleoptera, attack the roots. Corn root worm (Diabrotica undecimpunctata howardi Barber) for example is a particularly difficult pest to control or to eradicate. It attacks the plant below the soil line, where insecticides are difficult or impossible to apply effectively. Com root worm can be eradicated by root-specific expression of the Cry III gene, which produces a component, specifically toxic for coleoptera. Also root-specific expression of the limonene cyclase gene in combination with GPPP synthetase would be larvicidal. Another pest, nematodes, that penetrate the root and form root-knots or cysts, which cause severe damage to crops throughout the world, could be treated with the root-specific expression of nematode resistance genes such as Hs1^{pro-1}, Ni-1 and Gpa2.

In conclusion the invention disclosed here provides a method for conferring enhanced resistance to pathogens to a transgenic plant which pathogens attack the belowground plant tissue comprising the expression of a gene(s) of interest under the control of a transcriptional regulatory root-specific promoter as defined earlier.

In yet another embodiment of the invention, the invention provides a method to enhanced freezing tolerance comprising the expression of a gene(s) of interest under the control of a transcriptional regulatory root-specific promoter as defined earlier. Preferred is the overexpressing of the Arabidopsis thaliana DNA binding factors CBF1 and DREB1A that induce cold-regulated (COR) genes (Jaglo-Ottosen et al. 1998). However, constitutive expression of these genes in all organs of the plant led to growth retardation under normal growth conditions. Root-specific expression of these freezing-tolerance genes under control of the promoter sequence of the present invention can make the plant tolerant to cold temperatures of the soil without affecting the growth of the plant. This technique can have important agronomic potential since lots of soils are inaccessible for crop production because of presence of permanently frozen layers in the soil.

Alternatively, other genes of interest can tolerize the transgenic plant for other weather conditions like drought (e.g. oat arginin decarboxylase gene) and high salt conditions (e.g. MtID gene). In conclusion, the present invention provides a method to contribute to a transgenic plant stress-tolerance.

Further advantages to root-preferred gene expression include the production of useful proteins in an industrial setting. Light-sensitive proteins may be synthesized in root tissue such that said proteins are not exposed to light. Therefore the present invention also relates to a method for the production of light-sensitive proteins comprising the expression of a gene encoding said light-sensitive protein under the control of a transcriptional regulatory root-specific promoter of the invention.

Also considered as an embodiment of the invention is the use of the RCH1 promoter for applications based on the inhibition of expression of native DNA sequences within the plant's root to achieve a desired phenotype resulting from the silencing of the native gene. In this case, such inhibition might be accomplished with transformation of the plant to comprise a transcriptional regulatory root-specific promoter of the present invention operably linked to e.g. an antisense nucleotide sequence to the gene of interest, a gene silencing construct or a ribozyme.

Those skilled in the art will be aware that plants secure the formation of organs throughout their life span by developing and maintaining a collection of stem cells termed the meristem. The promoter of the present invention offers the opportunity to deliver a certain protein specifically to these cells. For example overexpression of genes that influence the proliferation and/or the differentiation of the root meristem could contribute to a higher turnover of the meristem cells and therefor enhance the root architecture.

The promoter of the present invention drives the Arabidopsis thaliana gene ROOT CLAVATA HOMOLOG 1 (RCH1). Since the clavata-genes are key role players in shoot-meristem maintenance, persons skilled in the art may recognize the possible involvement of the RCH1 operon in root meristem maintenance. Therefor one could assume that the control of this promoter is influenced by signals that promote meristem maintenance. This might be an advantage when aiming to stimulate the root meristem-formation, which eventually could result in more roots, higher uptake of resources and in higher crop yield.

Therefore, according to preferred embodiments, the invention relates to methods for stimulating meristem formation and/or maintaining root meristem comprising the expression of a gene that influences the proliferation and/or differentiation of the root mersiteme under the control of a transcriptional regulatory root-specific) promoter of the invention.

The present invention therefore relates to a method for stimulating root meristem formation or for root-meristeme maintenance comprising the expression in particular cells, tissues or organs of a plant, of a nucleic acid encoding an LRR receptor-like kinase, for instance the LRR receptor-like kinase of the invention, wherein optionally said nucleic acid is operably linked to a plant-operable promoter sequence, for instance said plant-operable promoter being the transcriptional regulatory root promoter as defined earlier.

As mentioned supra, the combined gene expression data and homology to CLAVATA 1 indicate that the novel plant LRR receptor-like kinase of the invention, RCH1 (SEQ ID No. 3, 4, 19), is very likely to exert a function that is involved in the development and/or maintenance of the root meristem. As the root is the main plant organ for nutrient uptake from the environment, enhanced root formation could eventually lead to overall growth enhancement and better yield. Therefore preferred genes to be expressed in the methods for stimulating meristem formation and/or for root meristeme maintenance are genes encoding the polypeptide(s) of the invention.

The present invention also relates to a method for enhancing root formation and/or root growth comprising the expression in particular cells, tissues or organs of a plant, of a nucleic acid encoding an LRR receptor-like kinase, for instance the LRR receptor-like kinase of the invention, wherein optionally said nucleic acid is operably linked to a plant-operable promoter sequence, for instance said plant-operable promoter being the transcriptional regulatory root promoter as defined earlier.

The present invention therefore relates to a method for enhancing overall growth and yield comprising the expression in particular cells, tissues or organs of a plant, of a nucleic acid encoding an LRR receptor-like kinase, for instance the LRR receptor-like kinase of the invention, wherein optionally said nucleic acid is operably linked to a plant-operable promoter sequence, for instance said plant-operable promoter being the transcriptional regulatory root promoter as defined earlier.

The present invention also relates to a method for enhancing overall growth and yield comprising the expression of a protein of the invention under the control of a transcriptional regulatory root (or root-specific) promoter of the invention.

Also in yet other embodiments of the invention, methods are provided for modifying cell fate and/or plant development and/or plant morphology and/or biochemistry and/or physiology comprising the modification of expression in particular cells, tissues or organs of a plant, of a nucleic acid encoding an LRR receptor-like kinase, for instance the LRR receptor-like kinase of the invention and defined above, wherein optionally said nucleic acid is operably linked to a plant-operable promoter sequence, for instance said plant-operable promoter being the transcriptional regulatory root promoter as defined earlier.

The present invention further relates to a method to confer pathogen resistance to a transgenic plant comprising the expression in particular cells, tissues or organs of a plant, of a nucleic acid encoding an LRR receptor-like kinase, for instance the LRR receptor-like kinase of the invention and defined above, wherein optionally said nucleic acid is operably linked to a plant-operable promoter sequence, for instance said plant-operable promoter being the transcriptional regulatory root promoter as defined earlier.

Many pathogen resistance genes are LRR receptor-like proteins, which initiate a signal transduction pathway in the root cells, leading to the activation of defense mechanisms against the pathogen. The novel LRR receptor-like kinase might contribute to pathogen resistance to the transgenic plant when transformed herein.

Yet another embodiment of the invention relates to methods to perform a functional analysis of the DNA sequence according to the present invention, encoding a novel LRR receptor-like protein. For persons skilled in the art it is possible to make single or double knock-out mutants of Arabidopsis thaliana, wherein the DNA of the present invention is inactivated. Also introduction of the antisense sequence of the DNA of the invention, as well as the overexpression of its sense sequence in a transgenic plant can elucidate a specific feature in this transgenic plant. Furthermore, the loss of function of this gene of the invention by introduction of dominant negative mutations can result in interesting characteristics of the mutant plant.

Another aspect of the present invention is the revelation of a new target for herbicides or growth regulators. When said gene of the invention is an essential gene for plant cell viability, it can be a target for a herbicide or growth regulator.

Therefore the invention relates to a method for identifying and obtaining proteins interacting with a (LRR receptor-like kinase) polypeptide of the invention comprising a screening assay wherein said polypeptide is used or expressed.

In a preferred embodiment the invention relates to a method for identifying and obtaining proteins interacting with a an LRR receptor-like kinase (protein) comprising a two-hybrid screening system wherein a nucleic acid encoding a polypeptide of the invention as a bait and a cDNA library as prey are expressed.

The invention also relates to a method for modulating the interaction between an LRR receptor-like kinase (protein) of the invention and interacting proteins obtainable by a method as described above.

The invention further relates to a method for identifying and obtaining compounds interacting with an LRR receptor-like kinase (protein) comprising the steps of:

a) providing a two-hybrid screening system wherein a polypeptide or protein of the invention and a protein interacting with said (LRR receptor-like kinase) polypeptide or protein or an interacting protein obtainable by a method of claim as claimed above are expressed,

b) interacting said compound with the complex formed by the expressed proteins as defined in a),

c) detecting a second complex, wherein the presence of said second complex identifies a compound which specifically binds to one of said polypeptides or said second complex, and

d) identifying the compound.

The invention also relates to a method for identifying compounds or mixtures of compounds which specifically bind to a polypeptide of the invention, comprising:

a) combining a polypeptide of the invention with said compound or mixtures of compounds under conditions suitable to allow complex formation, and,

b) detecting complex formation, wherein the presence of a complex identifies a molecule which specifically binds said polypeptide.

The invention further relates to the use of a molecule identified by means of one of the methods described above as a plant growth regulator or herbicide.

The invention also relates to a method for the production of a plant growth regulator or herbicide composition comprising the steps of one of the methods described above and formulating the compounds obtained from said steps in a suitable form for the application in agriculture or plant cell or tissue culture.

The invention also relates to the use of any of the nucleic acid molecules, vectors, polypeptides or antibodies of described herein for modifying cell fate and/or plant development and/or plant morphology and/or plant biochemistry and/or plant physiology.

The invention also relates to a diagnostic composition comprising at least one of the nucleic acid molecules, vectors, polypeptides or antibodies of the invention.

It is further important to notice that CLAVATA 1 has many analogues in other plant species, so it is likely that also the promoter of RCH1, the RCH1ORF or the complete RCH1 operon can be functional in those other plant species, and possibly exert the same specific activation pattern. This can be investigated by root-specific reporter gene expression in rice mediated by the Arabidopsis thaliana RCH1 promoter as described in example 5.

Definitions and Elaborations to the Embodiments

The terms “protein(s)”, “peptide(s)” or “oligopeptide(s)”, when used herein refer to amino acids in a polymeric form of any length. Said terms also include known amino acid modifications such as disulphide bond formation, cysteinylation, oxidation, glutathionylation, methylation, acetylation, farnesylation, biotinylation, stearoylation, formylation, lipoic acid addition, phosphorylation, sulphation, ubiquitination, myristoylation, palmitoylation, geranylgeranylation, cyclization (e.g. pyroglutamic acid formation), oxidation, deamidation, dehydration, glycosylation (e.g. pentoses, hexosamines, N-acetylhexosamines, deoxyhexoses, hexoses, sialic acid etc.), acylation and radiolabels (e.g. ¹²⁵I, ¹³¹I, ³⁵S, ¹⁴C, ³²P, ³³P, ³H) as well as non-naturally occurring amino acid residues, L-amino acid residues and D-amino acid residues.

“Homologues” or “Homologs” of a protein of the invention are those peptides, oligopeptides, polypeptides, proteins and enzymes which contain amino acid substitutions, deletions and/or additions relative to the said protein with respect to which they are a homolog, without altering one or more of its functional properties, in particular without reducing the activity of the resulting. For example, a homolog of said protein will consist of a bioactive amino acid sequence variant of said protein. To produce such homologs, amino acids present in the said protein can be replaced by other amino acids having similar properties, for example hydrophobicity, hydrophilicity, hydrophobic moment, antigenicity, propensity to form or break α-helical structures or β-sheet structures, and so on. An overview of physical and chemical properties of amino acids is given in Table 1.

Substitutional variants of a protein of the invention are those in which at least one residue in said protein amino acid sequence has been removed and a different residue inserted in its place. Amino acid substitutions ar typically of single residues, but may be clustered depending upon functional constraints placed upon the polypeptide; insertions will usually be of the order of about 1-10 amino acid residues and deletions will range from about 1-20 residues. Preferably, amino acid substitutions will comprise conservative amino acid substitutions, such as those described supra.

TABLE 1
Properties of naturally occurring amino acids.

| Charge properties/ | | |
|---|---|---|
| hydrophobicity | Side group | Amino Acid |
| nonpolar | Aliphatic | Ala, Ile, Leu, |
| hydrophobic | Aliphatic, | Val |
| | S-containing | |
| | Aromatic | Met |
| | Imino | Phe, Trp |
| | | Pro |
| polar uncharged | Aliphatic | Gly |
| | Amide | Asn, Gln |
| | Aromatic | Tyr |
| | Hydroxyl | Ser, Thr |
| | Sulfhydryl | Cys |
| positively charged | Basic | Arg, His, Lys |
| negatively charged | Acidic | Asp, Gly |

Insertional amino acid sequence variants of a protein of the invention are those in which one or more amino acid residues are introduced into a predetermined site in said protein. Insertions can comprise amino-terminal and/or carboxy-terminal fusions as well as intra-sequence insertions of single or multiple amino acids. Generally, insertions within the amino acid sequence will be smaller than amino or carboxyl terminal fusions, of the order of about 1 to 10 residues. Examples of amino- or carboxy-terminal fusion proteins or peptides include the binding domain or activation domain of a transcriptional activator as used in the yeast two-hybrid system, phage coat proteins, (histidine)₆-tag, glutathione S-transferase, protein A, maltose-binding protein, dihydrofolate reductase, Tag•100 epitope (EETARFQPGYRS), c-myc epitope (EQKLISEEDL), FLAG®-epitope (DYKDDDK), lacZ, CMP (calmodulin-binding peptide), HA epitope (YPYDVPDYA), protein C epitope (EDQVDPRLIDGK) and VSV epitope (YTDIEMNRLGK).

Deletional variants of a protein of the invention are characterised by the removal of one or more amino acids from the amino acid sequence of said protein.

Homologues of the peptides or polypeptides of the invention contain a number of amino acid substitutions, deletions and/or additions relative to said peptide or polypepthide such that said homologues are still substantially different from the peptides or polypeptides known in the art. The difference between proteins can be calculated in terms of % identidty according to methods well known in the art. Preferably, the homologues of the peptides or polypeptides of the present invention comprise the amino acid sequence represented in SEQ ID NO 5, optionally comprising minor amino acid substitutions such as those described in table 1. Other homologues of the polypeptides of the invention have an amino acid sequence which is at least 65% identical to the sequence represented in SEQ ID NO 4 or 19.

Amino acid variants of a protein of the invention may readily be made using peptide synthetic techniques well known in the art, such as solid phase peptide synthesis and the like, or by recombinant DNA manipulations. The manipulation of DNA sequences to produce variant proteins which manifest as substitutional, insertional or deletional variants are well known in the art. For example, techniques for making substitution mutations at predetermined sites in DNA having known sequence are well known to those skilled in the art, such as by M13 mutagenesis, T7-Gen in vitro mutagenesis kit (USB, Cleveland, Ohio), QuickChange Site Directed mutagenesis kit (Stratagene, San Diego, Calif.), PCR-mediated site-directed mutagenesis or other site-directed mutagenesis protocols.

“Derivatives” of a protein of the invention are those peptides, oligopeptides, polypeptides, proteins and enzymes which comprise at least about five contiguous amino acid residues of said polypeptide but which retain the biological activity of said protein and which may further comprise additional naturally-occurring, altered glycosylated, acylated or non-naturally occurring amino acid residues compared to the amino acid sequence of a naturally-occurring form of said polypeptide. Alternatively or in addition, a derivative may comprise one or more non-amino acid substituents compared to the amino acid sequence of a naturally-occurring form of said polypeptide, for example a reporter molecule or other ligand, covalently or non-covalently bound to the amino acid sequence such as, for example, a reporter molecule which is bound thereto to facilitate its detection. Preferably, the derivates of the proteins of the present invention comprise the amino acid acid sequence as represented in SEQ ID NO 5.

With “immunologically active” is meant that a molecule or specific fragments thereof such as epitopes or haptens are recognized by, i.e. bind to antibodies. Preferably, the immunologically active or fragments of the proteins of the present invention comprise the amino acid acid sequence as represented in SEQ ID NO 5.

With “functional fragment” or “functional homologue” is meant a protein which comprises at least about 5,10, 20, 50, 75, 100, 200, 300, 400, 500, 600, 700, 800, 900, 1000, 1050, 1100, 1110, 1120, 1130, 1131, 1132, 1133 or 1134 contiguous amino acid residues of said polypeptide but which retains the biological activity of said protein. Preferably, the functional fragments or functional homologues of the polypeptide or proteins of the invention comprise the amino acid sequence as represented in SEQ ID NO 5 but wherein minor amino acid substitutions are allowed (see table 1).

In the context of the current invention are embodied homologs, derivatives and/or immunologically active fragments of any of the inventive LRR receptor-like kinase protein or polypeptide or homolog, derivative or fragment thereof as defined supra.

“Antibodies” include monoclonal, polyclonal, synthetic or heavy chain camel antibodies as well as fragments of antibodies such as Fab, Fv or scFv fragments. Monoclonal antibodies can be prepared by the techniques as described in e.g. Liddle and Cryer (1991) which comprise the fusion of mouse myeloma cells to spleen cells derived from immunized animals. Furthermore, antibodies or fragments thereof to a molecule or fragments thereof can be obtained by using methods as described in e.g. Harlow and Lane (1988). In the case of antibodies directed against small peptides such as fragments of a protein of the invention, said peptides are generally coupled to a carrier protein before Immunization of animals. Such protein carriers include keyhole limpet hemocyanin (KLH), bovine serum albumin (BSA), ovalbumin and Tetanus toxoid. The carrier protein enhances the immune response of the animal and provides epitopes for T-cell receptor binding sites. The term “antibodies” furthermore includes derivatives thereof such as labelled antibodies. Antibody labels include alkaline phosphatase, PKH2, PKH26, PKH67, fluorescein (FITC), Hoecst 33258, R-phycoerythrin (PE), rhodamine (TRITC), Quantum Red, Texas Red, Cy3, biotin, agarose, peroxidase, gold spheres and radiolabels (e.g. ¹²⁵I, ¹³¹I, ³⁵S, ¹⁴C, ³²P, ³³P, ³H). Tools in molecular biology relying on antibodies against a protein include protein gel blot analysis, screening of expression libraries allowing gene identification, protein quantitative methods including ELISA and RIA, immunoaffinity purification of proteins, immunoprecipitation of proteins (e.g. Magyar et al. 1997) and immunolocalization. Other uses of antibodies and especially of peptide antibodies include the study of proteolytic processing (Loffler et al. 1994, Woulfe et al. 1994), determination of protein active sites (Lerner 1982), the study of precursor and post-translational processing (Baron and Baltimore 1982, Lerner et al. 1981, Semler et al. 1982), identification of protein domains involved in protein-protein interactions (Murakami et al. 1992) and the study of exon usage in gene expression (Tamura et al. 1991).

Embodied in the current invention are antibodies recognizing a LRR receptor-like kinase polypeptide or protein, or homolog, derivative or fragment thereof as defined supra or specific epitopes of said polypeptide or protein.

The terms “gene(s)”, “polynucleotide(s)”, “nucleic acid sequence(s)”, “nucleotide sequence(s)”, “DNA sequence(s)” or “nucleic acid molecule(s)”, when used herein refer to nucleotides, either ribonucleotides or deoxyribonucleotides or a combination of both, in a polymeric form of any length. Said terms furthermore include double-stranded and single-stranded DNA and RNA. Said terms also include known nucleotide modifications such as methylation, cyclization and ‘caps’ and substitution of one or more of the naturally occurring nucleotides with an analog such as inosine. Modifications of nucleotides include the addition of acridine, amine, biotin, cascade blue, cholesterol, Cy3®, Cy5®, Cy5.5® Dabcyl, digoxigenin, dinitrophenyl, Edans, 6-FAM, fluorescein, 3′-glyceryl, HEX, IRD-700, IRD-800, JOE, phosphate psoralen, rhodamine, ROX, thiol (SH), spacers, TAMRA, TET, AMCA-S®, SE, BODIPY®, Marina Blue®, Pacific Blue®, Oregon Green®, Rhodamine Greene®, Rhodamine Red®, Rhodol Green® and Texas Red®. Polynucleotide backbone modifications include methylphosphonate, 2′-OMe-methylphosphonate RNA, phosphorothiorate, RNA, 2′-OMeRNA. Base modifications include 2-amino-dA, 2-aminopurine, 3′-(ddA), 3′dA(cordycepin), 7-deaza-dA, 8-Br-dA, 8-oxo-dA, N⁶-Me-dA, abasic site (dSpacer), biotin dT, 2′-OMe-5Me-C, 2′-OMe-propynyl-C, 3′-(5-Me-dC), 3′-(ddC), 5-Br-dC, 5-1-dC, 5-Me-dC, 5-F-dC, carboxy-dT, convertible dA, convertible dC, convertible dG, convertible dT, convertible dU, 7-deaza-dG, 8-Br-dG, 8-oxo-dG, O⁶-Me-dG, S6-DNP-dG, 4-methyl-indole, 5-nitroindole, 2′-OMe-inosine, 2′-dl, O⁶-phenyl-di, 4-methyl-indole, 2′-deoxynebularine, 5-nitroindole, 2-aminopurine, dP(purine analogue), dK(pyrimidine analogue), 3-nitropyrrole, 2-thio-dT, 4-thio-dT, biotin-dT, carboxy-dT, O⁴-Me-dT, O⁴-triazol dT, 2′-OMe-propynyl-U, 5-Br-dU, 2′-dU, 5-F-dU, 5-1-dU, O⁴-triazol dU and radiolabels (e.g. ¹²⁵I, ¹³¹I, ³⁵S, ¹⁴C, ³²P, ³³P, ³H). Said terms also encompass peptide nucleic acids (PNAs), a DNA analogue in which the backbone is a pseudopeptide consisting of N-(2-aminoethyl)-glycine units rather than a sugar. PNAs mimic the behaviour of DNA and bind complementary nucleic acid strands. The neutral backbone of PNA results in stronger binding and greater specificity than normally achieved. In addition, the unique chemical, physical and biological properties of PNA have been exploited to produce powerful biomolecular tools, antisense and antigene agents, molecular probes and biosensors. With “recombinant DNA molecule” or “chimeric gene” is meant a hybrid DNA produced by joining pieces of DNA from different sources. With “heterologous nucleotide sequence” is intended a sequence that is not naturally occurring with the promoter sequence. While this nucleotide sequence is heterologous to the promoter sequence, it may be homologous, or native, or heterologous, or foreign, to the plant host. “Sense strand” refers to the strand of a double-stranded DNA molecule that is homologous to a mRNA transcript thereof. The “anti-sense strand” contains an inverted sequence which is complementary to that of the “sense strand”.

A “coding sequence” or “open reading frame” or “ORF” is defined as a nucleotide sequence that can be transcribed into mRNA and/or translated into a polypeptide when placed under the control of appropriate regulatory sequences, i.e. when said coding sequence or ORF is present in an expressible format. Said coding sequence of ORF is bounded by a 5′ translation start codon and a 3′ translation stop codon. A coding sequence or ORF can include, but is not limited to RNA, mRNA, cDNA, recombinant nucleotide sequences, synthetically manufactured nucleotide sequences or genomic DNA. Said coding sequence or ORF can be interrupted by intervening nucleic acid sequences.

TABLE 2
Degeneracy of the genetic code.

| | Three- | One- | |
|---|---|---|---|
| | letter | letter | |
| Amino Acid | code | code | Possible codons |
| Alanine | Ala | A | GCA GCC GCG GCU |
| Arginine | Arg | R | AGA AGG CGA CGC CGG CGU |
| Asparagine | Asn | N | AAC AAU |
| Aspartic Acid | Asp | D | GAC GAU |
| Cysteine | Cys | C | UGC UGU |
| Glutamic Acid | Glu | E | GAA GAG |
| Glutamine | Gln | Q | CAA CAG |
| Glycine | Gly | G | GGA GGC GGG GGU |
| Histidine | His | H | CAC CAU |
| Isoleucine | Ile | I | AUA AUG AUU |
| Leucine | Leu | L | UUA UUG CUA CUC CUG CUU |
| Lysine | Lys | K | AAA AAG |
| Methionine | Met | M | AUG |
| Phenylalanine | Phe | F | UUC UUU |
| Proline | Pro | P | CCA CCC CCG CCU |
| Serine | Ser | S | AGC AGU UCA UCA UCG UCU |
| Threonine | Thr | T | ACA ACC ACG ACU |
| Tryptophan | Trp | W | UGG |
| Tyrosine | Tyr | Y | UAC UAU |
| Valine | Val | V | GUA GUC GUG GUU |
| | | | Possible “STOP” codons |
| | | | UAA UAG UGA |

Genes and coding sequences essentially encoding the same protein but isolated from different sources can consist of substantially divergent nucleic acid sequences. Reciprocally, substantially divergent nucleic acid sequences can be designed to effect expression of essentially the same protein. Said nucleic acid sequences are the result of e.g. the existence of different alleles of a given gene, of the degeneracy of the genetic code or of differences in codon usage. Thus, as indicated in Table 2, amino acids such as methionine and tryptophan ar encoded by a single codon whereas other amino acids such as arginine, leucine and serine can each be translated from up to six different codons. Differences in preferred codon usage are illustrated below for Agrobacterium tumefaciens (a bacterium), A. thaliana, M. sativa (two dicotyledonous plants) and Oryza sativa (a monocotyledonous plant). These examples were extracted from (http://www.kazusa.or.jp/codon). To give one example, the codon GGC (for glycine) is the most frequently used codon in A. tumefaciens (36.2% ), is the second most frequently used codon in O. sativa but is used at much lower frequencies in A. thaliana and M. sativa (9% and 8.4%, respectively). Of the four possible codons encoding glycine (see Table 6), said GGC codon is most preferably used in A. tumefaciens and O. sativa. However, in A. thaliana this is the GGA (and GGU) codon whereas in M. sativa this is the GGU (and GGA) codon.

“Hybridization” is the process wherein substantially homologous complementary nucleotide sequences anneal to each other. The hybridization process can occur entirely is in solution, I.e. both complementary nucleic acids are in solution. Tools in molecular biology relying on such a process include the polymerase chain reaction (PCR; and all methods based thereon), subtractive hybridization, random primer extension, nuclease S1 mapping, primer extension, reverse transcription, cDNA synthesis, differential display of RNAs, and DNA sequence determination. The hybridization process can also occur with one of the complementary nucleic acids immobilized to a matrix such as magnetic beads, Sepharose beads or any other resin. Tools in molecular biology relying on such a process include the isolation of poly (A+) mRNA. The hybridization process can furthermore occur with one of the complementary nucleic acids immobilized to a solid support such as a nitrocellulose or nylon membrane or immobilized by e.g. photolitography to e.g. a silicious glass support (the latter known as nucleic acid arrays or microarrays or as nucleic acid chips). Tools in molecular biology relying on such a process include RNA and DNA gel blot analysis, colony hybridization, plaque hybridization, in situ hybridization and microarray hybridization. In order to allow hybridization to occur, the nucleic acid molecules are generally thermally or chemically denatured to melt a double strand into two single strands and/or to remove hairpins or other secondary structures from single stranded nucleic acids. The stringency of hybridization is influenced by conditions such as temperature, salt concentration and hybridization buffer composition. High stringency conditions for hybridization include high temperature and/or low salt concentration (salts include NaCl and Na₃-citrate) and/or the inclusion of formamide in the hybridization buffer and/or lowering the concentration of compounds such as SDS (detergent) in the hybridization buffer and/or exclusion of compounds such as dextran sulfate or polyethylene glycol (promoting molecular crowding) from the hybridization buffer. Conventional hybridization conditions are described in e.g. Sambrook et al. (1989) but the skilled craftsman will appreciate that numerous different hybridization conditions can be designed in function of the known or the expected homology and/or length of the nucleic acid sequence. Sufficiently low stringency hybridization conditions are particularly preferred to isolate nucleic acids heterologous to the DNA sequences of the invention defined supra. Elements contributing to said heterology include allelism, degeneration of the genetic code and differences in preferred codon usage as discussed supra.

Clearly, the current invention embodies the use of the inventive DNA sequences encoding a LRR receptor-like kinase, homolog, derivative and/or immunologically fragment thereof as defined higher in any method of hybridization. The current invention furthermore also relates to DNA sequences specifically hybridizing to said inventive DNA sequences.

DNA sequences as defined in the current invention can be interrupted by intervening sequences. With “intervening sequences” is meant any nucleic acid sequence which disrupts a coding sequence comprising said inventive DNA sequence or which disrupts the expressible format of a DNA sequence comprising said inventive DNA sequence. Removal of the intervening sequence restores said coding sequence or said expressible format. Examples of intervening sequences include introns, mobilizable DNA sequences such as transposons and DNA tags such as e.g. a T-DNA. With “mobilizable DNA sequence” is meant any DNA sequence that can be mobilized as the result of a recombination event.

To effect expression of a protein in a cell, tissue or organ, preferably of plant origin, either the protein may be introduced directly to said cell, such as by microinjection or ballistic means or alternatively, an isolated nucleic acid molecule encoding said protein may be introduced into said cell, tissue or organ in an expressible format.

Preferably, the DNA sequence of the invention comprises a coding sequence or open reading frame (ORF) encoding a LRR receptor-like kinase or a homolog or derivative thereof or an immunologically active fragment thereof as defined supra. The preferred protein of the invention comprises the amino acid sequence of said LRR receptor-like kinase.

With “vector” or “vector sequence” is meant a DNA sequence which can be introduced in an organism by transformation and can be stably maintained in said organism. Vector maintenance is possible in e.g. cultures of Escherichia coli, A. tumefaciens, Saccharomyces cerevisiae or Schizosaccharomyces pombe. Other vectors such as phagemids and cosmid vectors can be maintained and multiplied in bacteria and/or viruses. Vector sequences generally comprise a set of unique sites recognized by restriction enzymes, the multiple cloning site (MCS), wherein one or more non-vector sequence(s) can be inserted.

With “non-vector sequence” is accordingly meant a DNA sequence which is integrated in one or more of the sites of the MCS comprised within a vector.

“Expression vectors” form a subset of vectors which, by virtue of comprising the appropriate regulatory sequences enabling the creation of an expressible format for the inserted non-vector sequence(s), thus allowing expression of the protein encoded by said non-vector sequence(s). Expression vectors are known in the art enabling protein expression in organisms including bacteria (e.g. E. coli), fungi (e.g. S. cerevisiae, S. pombe, Pichia pastoris), insect cells (e.g. baculoviral expression vectors), animal cells (e.g. COS or CHO cells) and plant cells (e.g. potato virus X-based expression vectors, see e.g. Vance et al. 1998—WO9844097).

The current invention clearly includes any vector or expression vector comprising a non-vector DNA sequence comprising the promoter sequence according to the present invention or a non-vector sequence encoding a LRR receptor-like kinase, homolog, derivative and/or immunologically active fragment thereof as defined supra.

As an alternative to expression vector-mediated protein production in biological systems, chemical protein synthesis can be applied. Synthetic peptides can be manufactured in solution phase or in solid phase. Solid phase peptide synthesis (Merrifield 1963) is, however, the most common way and involves the sequential addition of amino acids to create a linear peptide chain. Solid phase peptide synthesis includes cycles consisting of three steps: (i) immobilization of the carboxy-terminal amino acid of the growing peptide chain to a solid support or resin; (ii) chain assembly, a process consisting of activation, coupling and deprotection of the amino acid to be added to the growing peptide chain; and (iii) cleavage involving removal of the completed peptide chain from the resin and removal of the protecting groups from the amino acid side chains. Common approaches in solid phase peptide synthesis include Fmoc/tBu (9-fluorenylmethyloxycarbonyl/t-butyl) and Boc (t-butyloxycarbonyl) as the amino-terminal protecting groups of amino acids. Amino acid side chain protecting groups include methyl (Me), formyl (CHO), ethyl (Et), acetyl (Ac), t-butyl (t-Bu), anisyl, benzyl (Bzl), trifluroacetyl (Tfa), N-hydroxysuccinimide (ONSu, OSu), benzoyl (Bz), 4-methylbenzyl (Meb), thioanizyl, thiocresyl, benzyloxymethyl (Bom), 4-nitrophenyl (ONp), benzyloxycarbonyl (Z), 2-nitrobenzoyl (NBz), 2-nitrophenylsulphenyl (Nps), 4-toluenesulphonyl (Tosyl, Tos), pentafluorophenyl (Pfp), diphenylmethyl (Dpm), 2-chlorobenzyloxycarbonyl (CI-Z), 2,4,5-trichlorophenyl, 2-bromobenzyloxycarbonyl (Br-Z), tripheylmethyl (Trityl, Trt), and 2,5,7,8-pentamethylchroman-6-sulphonyl (Pmc). During chain assembly, Fmoc or Boc are removed resulting in an activated amino-terminus of the amino acid residue bound to the growing chain. The carboxy-terminus of the incoming amino acid is activated by conversion into a highly reactive ester, e.g. by HBTU. With current technologies (e.g. PerSeptive Biosystems 9050 synthesizer, Applied Biosystems Model 431 A Peptide Synthesizer), linear peptides of up to 50 residues can be manufactured. A number of guidelines is available to produce peptides that are suitable for use in biological systems including (i) limiting the use of difficult amino acids such as cys, met, trp (easily oxidized and/or degraded during peptide synthesis) or arg; (ii) minimize hydrophobic amino acids (can impair peptide solubility); and (iii) prevent an amino-terminal glutamic acid (can cyclize to pyroglutamate).

By “expressible format” is meant that the isolated nucleic acid molecule is in a form suitable for being transcribed into mRNA and/or translated to produce a protein, either constitutively or following induction by an intracellular or extracellular signal, such as an environmental stimulus or stress (mitogens, anoxia, hypoxia, temperature, salt, light, dehydration, etc) or a chemical compound such as IPTG (isopropyl-β-D-thiogalactopyranoside) or such as an antibiotic (tetracycline, ampicillin, rifampicin, kanamycin), hormone (e.g. gibberellin, auxin, cytokinin, glucocorticoid, brassinosteroid, ethylene, abscisic acid etc), hormone analogue (iodoacetic acid (IAA), 2,4D, etc), metal (zinc, copper, iron, etc), or dexamethasone, amongst others. As will be known to those skilled in the art, expression of a functional protein may also require one or more post-translational modifications, such as glycosylation, phosphorylation, dephosphorylation, or one or more protein-protein interactions, amongst others. All such processes are included within the scope of the term “expressible format”.

Preferably, expression of a protein in a specific cell, tissue, or organ, preferably of plant origin, is effected by introducing and expressing an isolated nucleic acid molecule encoding said protein, such as a cDNA molecule, genomic gene, synthetic oligonucleotide molecule, mRNA molecule or open reading frame, to said cell, tissue or organ, wherein said nucleic acid molecule is placed operably in connection with suitable regulatory sequences including a promoter, preferably a plant-expressible promoter, and a terminator sequence.

“Regulatory sequence” refers to control DNA sequences which are necessary to affect the expression of coding sequences to which they are ligated. The nature of such control sequences differs depending upon the host organism. In prokaryotes, control sequences generally include promoters, ribosomal binding sites, and terminators. In eukaryotes generally control sequences include promoters, terminators and enhancers or silencers. The term “control sequence” is intended to include, at a minimum, all components the presence of which are necessary for expression, and may also include additional advantageous components and which determines when, how much and where a specific gene is expressed.

Reference herein to a “promoter” is to be taken in its broadest context and includes the transcriptional regulatory sequences derived from a classical eukaryotic genomic gene, including the TATA box which is required for accurate transcription initiation, with or without a CCAAT box sequence and additional regulatory elements (i.e. upstream activating sequences, enhancers and silencers) which alter gene expression in response to developmental and/or external stimuli, or in a tissue-specific manner.

The term “promoter” also includes the transcriptional regulatory sequences of a classical prokaryotic gene, in which case it may include a −35 box sequence and/or a −10 box transcriptional regulatory sequences.

The term “promoter” is also used to describe a synthetic or fusion molecule, or derivative which confers, activates or enhances expression of a nucleic acid molecule in a cell, tissue or organ.

The term “functional part” when referred to nucleic acids in relation to the above defined transcriptional regulatory sequences, refers to a part or parts of the nucleic acid having the activity to specifically drive or promote transcription from sequences which are located downstream of said nucleic acid sequence.

Promoters may contain additional copies of one or more specific regulatory elements, to further enhance expression and/or to alter the spatial expression and/or temporal expression of a nucleic acid molecule to which it is operably connected. Such regulatory elements may be placed adjacent to a heterologous promoter sequence to drive expression of a nucleic acid molecule in response to e.g. copper, glucocorticoids, dexamethasone, tetracycline, gibberellin, cAMP, abscisic acid, auxin, wounding, ethylene, jasmonate or salicylic acid or to confer expression of a nucleic acid molecule to specific cells, tissues or organs such as meristems, leaves, roots, embryo, flowers, seeds or fruits.

In the context of the present invention, the promoter preferably is a plant-expressible promoter sequence. Promoters, however, that also function or solely function in non-plant cells such as bacteria, yeast cells, insect cells and animal cells are not excluded from the invention. By “plant-expressible” is meant that the promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ. The terms “plant-operable” and “operable in a plant” when used herein, in respect of a promoter sequence, shall be taken to be equivalent to a plant-expressible promoter sequence.

In the present context, a “regulatable promoter sequence” is a promoter that is capable of conferring expression on a structural gene in a particular cell, tissue, or organ or group of cells, tissues or organs of a plant, optionally under specific conditions, however does generally not confer expression throughout the plant under all conditions. Accordingly, a regulatable promoter sequence may be a promoter sequence that confers expression on a gene to which it is operably connected in a particular location within the plant or alternatively, throughout the plant under a specific set of conditions, such as following induction of gene expression by a chemical compound or other elicitor.

Preferably, the regulatable promoter used in the performance of the present invention confers expression in a specific location within the plant, either constitutively or following induction, however not in the whole plant under any circumstances. Included within the scope of such promoters are cell-specific promoter sequences, tissue-specific promoter sequences, organ-specific promoter sequences, cell cycle specific gene promoter sequences, inducible promoter sequences and constitutive promoter sequences that have been modified to confer expression in a particular part of the plant at any one time, such as by integration of said constitutive promoter within a transposable genetic element (Ac, Ds, Spm, En, or other transposon). The skilled craftsman will understand that a “constitutive promoter” is a promoter that is transcriptionaly active in an organism, preferably a plant, during most, but not necessarily all phases of its growth and development. Similarly, the skilled craftsman will understand that a “ubiquitous promoter” is a promoter that is transcriptionally active throughout most, but not necessarily all parts of an organism, preferably a plant.

Generally by “weak promoter” is intended a promoter that drives expression of a coding sequence at a low level. By “low level” is intended at levels of about 1/10,000 transcripts to about 1/100,000 transcripts, to about 1/500,0000 transcripts. Conversely, a “strong promoter” drives expression of a coding sequence at high level, or at about 1/10 transcripts to about 1/100 transcripts to about 1/1,000 transcripts.

The term “cell-specific” shall be taken to indicate that expression is predominantly in a particular cell or cell-type, preferably of plant origin, albeit not necessarily exclusively in said cell or cell-type.

Similarly, the term “tissue-specific” shall be taken to indicate that expression is predominantly in a particular tissue or tissue-type, preferably of plant origin, albeit not necessarily exclusively in said tissue or tissue-type.

Similarly, the term “organ-specific” shall be taken to indicate that expression is predominantly in a particular organ, preferably of plant origin, albeit not necessarily exclusively in said organ. “Root specific” means that the promoter is expressed in the root only and not in other tissues of the plant.

By “root-preferred” it is intended that expression of the heterologous nucleotide sequence is most abundant root, but could also have low expression levels elsewhere in the plant. While some level of expression of the heterologous nucleotide sequence occurs in other plant tissue types, expression occurs most abundantly in the root including primary, lateral and adventitious roots.

By “root” is intended any part of the root structure, including, but not limited to, the root cap, apical meristem, protoderm, ground meristem, procambium, endodermis, cortex, vascular cortex, epidermis, and the like.

Similarly, the term “cell cycle specific” shall be taken to indicate that expression is predominantly cyclic and occurring in one or more, not necessarily consecutive phases of the cell cycle albeit not necessarily exclusively in cycling cells, preferably of plant origin.

Contrarily, the term ubiquitous shall be taken to indicate that expression is throughout the entire oranism.

Those skilled in the art will be aware that an “inducible promoter” is a promoter the transcriptional activity of which is increased or induced in response to a developmental, chemical, environmental, or physical stimulus. Similarly, the skilled craftsman will understand that a “constitutive promoter” is a promoter that is transcriptionally active throughout most, but not necessarily all parts of an organism, preferably a plant, during most, but not necessarily all phases of its growth and development.

Those skilled in the art will readily be capable of selecting appropriate promoter sequences for use in regulating appropriate expression of the LRR receptor-like kinase as described supra from publicly-available or readily-available sources, without undue experimentation.

Placing a nucleic acid molecule under the regulatory control of a promoter sequence, or in operable connection with a promoter sequence, means positioning said nucleic acid molecule such that expression is controlled by the promoter sequence. A promoter is usually, but not necessarily, positioned upstream, or at the 5′-end, and within 2 kb of the start site of transcription, of the nucleic acid molecule which it regulates. In the construction of heterologous promoter/structural gene combinations it is generally preferred to position the promoter at a distance from the gene transcription start site that is approximately the same as the distance between that promoter and the gene it controls in its natural setting (i.e., the gene from which the promoter is derived). As is known in the art, some variation in this distance can be accommodated without loss of promoter function. Similarly, the preferred positioning of a regulatory sequence element with respect to a heterologous gene to be placed under its control is defined by the positioning of the element in its natural setting (i.e., the gene from which it is derived). Again, as is known in the art, some variation in this distance can also occur.

“Expression” means the production of a protein or nucleotide sequence in the cell itself or in a cell-free system. It includes transcription into an RNA product, post-transcriptional modification and/or translation to a protein product or polypeptide from a DNA encoding that product, as well as possible post-translational modifications.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In case the control sequence is a promoter, R is obvious for a skilled person that double-stranded nucleic acid is preferably used.

Examples of promoters suitable for use in gene constructs of the present invention include those listed in Table 3, amongst others. The promoters listed in Table 5 are provided for the purposes of exemplification only and the present invention is not to be limited by the list provided therein. Those skilled in the art will readily be in a position to provide additional promoters that are useful in performing the present invention.

In the case of constitutive promoters or promoters that induce expression throughout the entire plant, it is preferred that such sequences are modified by the addition of nucleotide sequences derived from one or more of the tissue-specific promoters listed in Table 8, or alternatively, nucleotide sequences derived from one or more of the above-mentioned tissue-specific inducible promoters, to confer tissue-specificity thereon. For example, the CaMV 35S promoter may be modified by the addition of maize Adh1 promoter sequence, to confer anaerobically-regulated root-specific expression thereon, as described previously (Ellis et al, 1987). Another example describes conferring root specific or root abundant gene expression by fusing the CaMV35S promoter to elements of the maize glycine-rich protein GRP3 gene (Feix and Wulff 2000—WO0015662). Such modifications can be achieved by routine experimentation by those skilled in the art.

The term “terminator” refers to a DNA sequence at the end of a transcriptional unit which signals termination of transcription. Terminators are 3′-non-translated DNA sequences containing a polyadenylation signal, which facilitates the addition of polyadenylate sequences to the 3′-end of a primary transcript. Terminators active in cells derived from viruses, yeasts, moulds, bacteria, insects, birds, mammals and plants are known and described in the literature. They may be isolated from bacteria, fungi, viruses, animals and/or plants.

Examples of terminators particularly suitable for use in the gene constructs of the present invention include the Agrobacterium tumefaciens nopaline synthase (NOS) gene terminator, the Agrobacterium tumefaciens octopine synthase (OCS) gene terminator sequence, the Cauliflower mosaic virus (CaMV) 35S gene terminator sequence, the Oryza sativa ADP-glucose pyrophosphorylase terminator sequence (t3′Bt2), the Zea mays zein gene terminator sequence, the rbcs-1A gene terminator, and the rbcs-3A gene terminator sequences, amongst others.

TABLE 3
Exemplary plant-expressible promoters for use in the performance of the present invention

| | | |
|---|---|---|
| | EXPRESSION | |
| GENE SOURCE | PATTERN | REFERENCE |

I: CELL-SPECIFIC, TISSUE-SPECIFIC, AND ORGAN-SPECIFIC PROMOTERS

| | | |
|---|---|---|
| α-amylase (Amy32b) | Aleurone | Lanahan et al, Plant Cell 4: 203-211, 1992; |
| | | Skriver et al, Proc Natl Acad Sci USA |
| | | 88: 7266-7270, 1991 |
| cathepsin β-like | Aleurone | Cejudo et al, Plant Mol Biol 20: 849-856, |
| gene | | 1992 |
| Agrobacterium | Cambium | Nilsson et al, Physiol Plant 100: 456-462, |
| rhizogenes rolB | | 1997 |
| AtPRP4 | Flowers | http://salus.medium.edu/mmg/tiernev/html |
| chalcone synthase | Flowers | Van der Meer et al, Plant Mol Biol 15: 95-109, 1990 |
| (chsA) | | |
| LAT52 | Anther | Twell et al, Mol Gen Genet 217: 240-245, |
| | | 1989 |
| apetala-3 | Flowers | |
| chitinase | fruit (berries, grapes, | Thomas et al. CSIRO Plant Industry, |
| | etc) | Urrbrae, South Australia, Australia; |
| | | http://winetitles.com.au/gwrdc/csh95-1.html |
| rbcs-3A | green tissue (eg leaf) | Lam et al, Plant Cell 2: 857-866, 1990; |
| | | Tucker et al., Plant Physiol 113: 1303-1308, |
| | | 1992 |
| leaf-specific genes | Leaf | Baszczynski et al, Nucl Acid Res 16: 4732, |
| | | 1988 |
| AtPRP4 | Leaf | http://salus.medium.edu/mmg/tierney/html |
| chlorella virus | Leaf | Mitra and Higgins, Plant Mol Biol 26: 85-93, 1994 |
| adenine | | |
| methyltransferase | | |
| gene promoter | | |
| aldP gene promoter | Leaf | Kagaya et al, Mol Gen Genet 248: 668-674, |
| from rice | | 1995 |
| rbcs promoter from | Leaf | Kyozuka et al, Plant Physiol 102: 991-1000, |
| rice or tomato | | 1993 |
| Pinus cab-6 | Leaf | Yamamoto et al, Plant Cell Physiol 35: 773-778, |
| | | 1994 |
| rubisco promoter | Leaf | |
| cab (chlorophyll | Leaf | |
| a/b/binding protein | | |
| pea Blec4 gene | Vegetative and floral | Mandaci and Dobres, Plant Mol Biol |
| | epidermal tissues | 34: 961-965 |
| SAM22 | senescent leaf | Crowell et al, Plant Mol Biol 18: 459-466, |
| | | 1992 |
| ltp gene (lipid | | Fleming et al, Plant J 2: 855-862, 1992 |
| transfer gene) | | |
| R. japonicum nif | nodule | U.S. Pat. No. 4 803165 |
| gene | | |
| B. japonicum nifH | nodule | U.S. Pat. No. 5008194 |
| gene | | |
| GmENOD40 | nodule | Yang et al, Plant J 3: 573-585, 1993 |
| PEP carboxylase | nodule | Pathirana et al, Plant Mol Biol 20: 437-450, |
| (PEPC) | | 1992 |
| Leghaemoglobin | nodule | Gordon et al, J Exp Bot 44: 1453-1465, |
| (Lb) | | 1993 |
| Tungro bacilliform | phloem | Bhattacharyya-Pakrasi et al, Plant J 4: 71-79, |
| virus gene | | 1992 |
| pollen-specific genes | pollen; microspore | Albani et al, Plant Mol Biol 15: 605, 1990; |
| | | Albani et al, Plant Mol Biol 16: 501, 1991 |
| Zm13 | pollen | Guerrero et al, Mol Gen Genet 224: 161-168, |
| | | 1993 |
| apg gene | microspore | Twell et al, Sex Plant Reprod 6: 217-224, |
| | | 1993 |
| maize pollen-specific | pollen | Hamilton et al, Plant Mol Biol 18: 211-218, |
| gene | | 1992 |
| Sunflower pollen- | pollen | Baltz et al, Plant J 2: 713-721, 1992 |
| expressed gene | | |
| B. napus pollen- | pollen;anther; tapetum | Arnoldo et al, J Cell Bioch m, Abstract No. |
| specific gene | | Y101, 204, 1992 |
| root-expressible | roots | Tingey et al, EMBO J 6: 1, 1987 |
| genes | | |
| Tobacco auxin- | root tip | Van der Zaal et al, Plant Mol Biol 16: 983, |
| inducible gene | | 1991 |
| β-tubulin | root | Oppenheimer et al, Gene 63: 87, 1988 |
| Tobacco root- | root | Conkling et al, Plant Physiol 93: 1203, 1990 |
| specific genes | | |
| B. napus G1-3b | root | U.S. Pat. No. 5401836 |
| gene | | |
| SbPRP1 | roots | Suzuki et al, Plant Mol Biol 21: 109-119, |
| | | 1993 |
| AtPRP1; AtPRP3 | roots; root hairs | http://salus.medium.edu/mmg/tierney/html |
| RD2 gene | root cortex | http://www2.cnsu.edu/ncsu/research |
| TobRB7 gene | root vasculature | http://www2.cnsu.edu/ncsu/research |
| AtPRP4 | leaves; flowers; lateral | http://salus.medium.edu/mmg/tierney/html |
| | root primordia | |
| seed-specific genes | seed | Simon et al, Plant Mol Biol 5: 191, 1985; |
| | | Scofield et al, J Biol Chem 262: 12202, |
| | | 1987; Baszczynski et al, Plant Mol Biol |
| | | 14: 633, 1990 |
| Brazil Nut albumin | seed | Pearson et al, Plant Mol Biol 18: 235-245, |
| | | 1992 |
| Legumin | seed | Ellis et al, Plant Mol Biol 10: 203-214, 1988 |
| Glutelin (rice) | seed | Takaiwa et al, Mol Gen Genet 208: 15-22, |
| | | 1986; Takaiwa et al, FEBS Lett 221: 43-47, |
| | | 1987 |
| Zein | seed | Matzke et al, Plant Mol Biol 14: 323-32 1990 |
| NapA | seed | Stalberg et al, Planta 199: 515-519, 1996 |
| wheat LMW and | endosperm | Mol Gen Genet 216: 81-90, 1989; Nucl |
| HMW glutenin-1 | | Acids Res 17: 461-462, 1989 |
| wheat SPA | seed | Albani et al, Plant Cell 9: 171-184, 1997 |
| cZ19B1, maize 19 | seed | WO0011177 |
| kDa zein | | |
| mi1ps, maiz | seed | WO0011177 |
| myoinositol-1-Pi | | |
| synthase | | |
| wheat α, β, γ-gliadins | endosperm | EMBO J 3: 1409-1415, 1984 |
| barley ltr1 promoter | endosperm | |
| barley B1, C, D, | endosperm | Theor Appl Gen 98: 1253-1262, 1999; Plant |
| hordein | | J 4: 343-355, 1993; Mol Gen Genet |
| | | 250: 750-60, 1996 |
| barley DOF | endosperm | Mena et al, Plant J 116: 53-62, 1998 |
| blz2 | endosperm | EP99106056.7 |
| synthetic promoter | endospem | Vicente-Carbajosa et al, Plant J 13: 629-640, |
| | | 1998 |
| rice prolamin NRP33 | endosperm | Wu et al, Plant Cell Physiol 39: 885-889, |
| | | 1998 |
| rice α-globulin Glb-1 | endosperm | Wu et al, Plant Cell Physiol 39: 885-889, |
| | | 1998 |
| maize END genes | endosperm | WO0012733 |
| barley END1 | endosperm | WO9808961 |
| barley NUC1 | nucellus | WO9808961 |
| rice OSH1 | embryo | Sato et al, Proc Natl Acad Sci USA |
| | | 93: 8117-8122, 1996 |
| rice α-globulin | endosperm | Nakase et al, Plant Mol Biol 33: 513-522, |
| REB/OHP-1 | | 1997 |
| rice ADP-glucose PP | endosperm | Trans Res 6: 157-168, 1997 |
| maize ESR gene | endosperm | Plant J 12: 235-246, 1997 |
| family | | |
| sorgum γ-kafirin | endosperm | Plant Mol Biol 32: 1029-1035, 1996 |
| KNOX | embryo | Postma-Haarsma et al, Plant Mol Biol |
| | | 39: 257-271, 1999 |
| rice oleosin | embryo and aleuron | Wu et al, J Biochem 123: 386, 1998 |
| sunflower oleosin | seed (embryo and dry | Cummins et al, Plant Mol Biol 19: 873-876, |
| | seed) | 1992 |
| LEAFY | shoot meristem | Weigel et al, Cell 69: 843-859, 1992 |
| Arabidopsis thaliana | shoot meristem | Accession number AJ131822 |
| knat1 | | |
| Malus domestica | shoot meristem | Accession number Z71981 |
| kn1 | | |
| CLAVATA1 | shoot meristem | Accession number AF049870 |
| stigma-specific | stigma | Nasrallah et al, Proc Natl Acad Sci USA |
| genes | | 85: 5551, 1988; Trick et al, Plant Mol Biol |
| | | 15: 203, 1990 |
| class I patatin gene | tuber | Liu et al, Plant Mol Biol 153: 386-395, 1991 |
| PCNA rice | meristem | Kosugi et al, Nucl Acids Res 19: 1571-1576, |
| | | 1991; Kosugi and Ohashi, Plant Cell |
| | | 9: 1607-1619, 1997 |
| Pea TubA1 tubulin | Dividing cells | Stotz and Long, Plant Mol Biol 41: 601-614, |
| | | 1999 |
| Arabidopsis cdc2a | cycling cells | Chung and Parish, FEBS Lett 362: 215-219, |
| | | 1995 |
| Arabidopsis Rop1A | Anthers; mature pollen + pollen | Li et al, Plant Physiol 118: 407-417, 1998 |
| | tubes | |
| Arabidopsis AtDMC1 | Meiosis-associated | Klimyuk and Jones, Plant J 11: 1-14, 1997 |
| Pea PS-IAA4/5 and | Auxin-inducible | Wong et al, Plant J 9: 587-599, 1996 |
| PS-IAA6 | | |
| Pea | Meristematic tissues; | Zhou et al, Plant J 12: 921-930, 1997 |
| famesyltransferase | phloem near growing | |
| | tissues; light-and | |
| | sugar-repressed | |
| Tobacco (N. | Dividing cells/ | Trehin et al, Plant Mol. Biol. 35: 667-672, |
| sylvestris) cyclin | meristematic tissue | 1997 |
| B1;1 | | |
| Catharanthus roseus | Dividing cells/ | Ito et al, Plant J 11: 983-992, 1997 |
| Mitotic cyclins CYS | meristematic tissue | |
| (A-type) and CYM | | |
| (B-type) | | |
| Arabidopsis cyc1At | Dividing cells/ | Shaul et al, Proc Natl Acad Sci USA |
| (=cyc B1;1) and | meristematic tissue | 93: 4868-4872, 1996 |
| cyc3aAt (A-type) | | |
| Arabidopsis tef1 | Dividing cells/ | Regad et al, Mol Gen Genet 248: 703-711, |
| promoter box | meristematic tissue | 1995 |
| Catharanthus roseus | Dividing cells/ | Ito et al, Plant Mol Biol 24: 863-878, 1994 |
| cyc07 | meristematic tissue | |

II: EXEMPLARY CONSTITUTIVE PROMOTERS

| | | |
|---|---|---|
| Actin | constitutive | McElroy et al, Plant Cell 2: 163-171, 1990 |
| CAMV 35S | constitutive | Odell et al, Nature 313: 810-812, 1985 |
| CaMV 19S | constitutive | Nilsson et al, Physiol Plant 100: 456-462, |
| | | 1997 |
| GOS2 | constitutive | de Pater et al, Plant J 2: 837-844, 1992 |
| Ubiquitin | constitutive | Christensen et al, Plant Mol Biol 18: 675-689, |
| | | 1992 |
| rice cyclophilin | constitutive | Buchholz et al, Plant Mol Biol 25: 837-843, |
| | | 1994 |
| maize histone H3 | constitutive | Lepetit et al, Mol Gen Genet 231: 276-285, |
| | | 1992 |
| alfalfa histone H3 | constitutive | Wu et al, Nucleic Acids Res 17: 3057-3063, |
| | | 1989; Wu et al, Plant Mol Biol 11: 641-649, |
| | | 1988 |
| actin 2 | constitutive | An et al, Plant J 10: 107-121, 1996 |

III: EXEMPLARY STRESS-INDUCIBLE PROMOTERS

| NAME | STRESS | REFERENCE |
|---|---|---|
| P5CS (delta(1)- | salt, water | Zhang et al, Plant Sci 129: 81-89, 1997 |
| pyrroline-5- | | |
| carboxylate syntase) | | |
| cor15a | cold | Hajela et al, Plant Physiol 93: 1246-1252, |
| | | 1990 |
| cor15b | cold | Wlihelm et al, Plant Mol Biol 23: 1073-1077, |
| | | 1993 |
| cor15a (−305 to +78 | cold, drought | Baker et al, Plant Mol Biol 24: 01-713, 1994 |
| nt) | | |
| rd29 | salt, drought, cold | Kasuga et al, Nature Biotechnol 18: 287-291, |
| | | 1999 |
| heat shock proteins, | heat | Barros et al, Plant Mol Biol 19 665-75, |
| Including artificial | | 1992. Marrs et al, Dev Genet14: 27-41, |
| promoters containing | | 1993. Schoffl et al, Mol Gen Genet |
| the heat shock | | 217: 246-53, 1989. |
| element (HSE) | | |
| SmHSP (small heat | heat | Waters et al, J Exp Bot 47: 325-338, 1996 |
| shock proteins) | | |
| wcs120 | cold | Ouellete et al, FEBS Lett 423: 324-328, |
| | | 1998 |
| ci7 | cold | Kirch et al, Plant Mol Biol 33: 897-909, |
| | | 1997 |
| Adh | cold, drought, hypoxia | Dolferus et al, Plant Physiol 105: 1075-87, |
| | | 1994 |
| pwsi18 | salt and drought | Joshee et al, Plant Cell Physiol 39: 64-72, |
| | | 1998 |
| ci21A | cold | Schneider et al, Plant Physiol 113: 335-45, |
| | | 1997 |
| Trg-31 | drought | Chaudhary et al, Plant Mol Biol 30: 1247-57, |
| | | 1996 |
| Osmotin | osmotic | Raghothama et al, Plant Mol Biol 23: 1117-28, |
| | | 1993 |
| LapA | wounding, | WO99/03977 University of California/INRA |
| | enviromental | |

IV: EXEMPLARY PATHOGEN-INDUCIBLE PROMOTERS

| NAME | PATHOGEN | REFERENCE |
|---|---|---|
| RB7 | Root-knot nematodes | US5760386 - North Carolina State |
| | (Meloidogyne spp.) | University; Opperman et al, Science |
| | | 263: 221-23, 1994 |
| PR-1, 2, 3, 4, 5, 8, 11 | fungal, viral, bacterial | Ward et al, Plant Cell 3: 1085-1094, 1991; |
| | | Reiss et al 1996; Label et al, Plant J |
| | | 16: 223-233, 1998; Melchers et al, Plant J |
| | | 5: 469-480, 1994; Lawton et al, Plant Mol |
| | | Biol, 19: 735-743, 1992 |
| HMG2 | nematodes | WO9503690 - Virginia Tech Intellectual |
| | | Properties Inc. |
| Abi3 | Cyst nematodes | unpublished |
| | (Heterodera spp.) | |
| ARM1 | nematodes | Barthels et al, Plant Cell 9: 2119-2134, 1997 |
| | | WO 98/31822 - Plant Genetic Systems |
| Att0728 | nematodes | Barthels et al, Plant Cell 9: 2119-2134, |
| | | 1997 |
| | | PCT/EP98/07761 |
| Att1712 | nematodes | Barthels et al, Plant Cell 9, 2119-2134, |
| | | 1997 |
| | | PCT/EP98/07761 |
| Gst1 | Different types of | Strittmatter et al, Mol Plant-Microbe Interact |
| | pathogens | 9: 68-73, 1996 |
| LEMMI | nematodes | WO 92/21757 - Plant Genetic Systems |
| CLE | geminivirus | PCT/EP99/03445 - CINESTAV |
| PDF1.2 | Fungal including | Manners et al, Plant Mol Biol, 38: 1071-1080, |
| | Alternaria brassicicola | 1998 |
| | and Botrytis cinerea | |
| Thi2.1 | Fungal - Fusarium | Vignutelli et al, Plant J 14: 285-295, 1998 |
| | oxysporum f sp. | |
| | matthiolae | |
| DB#226 | nematodes | Bird and Wilson, Mol Plant-Microbe Interact |
| | | 7: 419-442, 1994 |
| | | WO 95.322888 |
| DB#280 | nematodes | Bird and Wilson, Mol Plant-Microbe Interact |
| | | 7: 419-442, 1994 |
| | | WO 95.322888 |
| Cat2 | nematodes | Niebel et al, Mol Plant-Microbe Interact |
| | | 8: 371-378, 1995 |
| □Tub | nematodes | Aristizabal et al (1996), 8^{th} International |
| | | Congress on Plant-Microbe Interaction, |
| | | Knoxville US B-29 |
| SHSP | nematodes | Fenoll et al (1997) In: Cellular and |
| | | molecular aspects of plant-nematode |
| | | interactions. Kluwer Academic, C. Fenoll, |
| | | F. M. W. Grundler and S.A. Ohl (Eds.), |
| Tsw12 | nematodes | Fenoll et al (1997) In: Cellular and |
| | | molecular aspects of plant-nematode |
| | | Interactions. Kluwer Academic, C. Fenoll, |
| | | F. M. W. Grundler and S.A. Ohl (Eds.) |
| Hs1(pro1) | nematodes | WO 98/122335 - Jung |
| NsLTP | viral, fungal, bacterial | Molina and Garcia-Olmedo FEBS Lett, |
| | | 316: 119-122, 1993 |
| RIP | viral, fungal | Tumer et al, Proc Natl Acad Sci USA |
| | | 94: 3866-3871, 1997 |

Those skilled in the art will be aware of additional promoter sequences and terminator sequences which may be suitable for use in performing the invention. Such sequences may readily be used without any undue experimentation.

In the context of the current invention, “ectopic expression” or “ectopic overexpression” of a gene or a protein are conferring to expression patterns and/or expression levels of said gene or protein normally not occurring under natural conditions. Ectopic expression can be achieved in a number of ways including operably linking of a coding sequence encoding said protein to an isolated homologous or heterologous promoter in order to create a chimeric gene and/or operably linking said coding sequence to its own isolated promoter (i.e. the unisolated promoter naturally driving expression of said protein) in order to create a recombinant gene duplication or gene multiplication effect. With “ectopic co-expression” is meant the ectopic expression or ectopic overexpression of two or more genes or proteins. The same or, more preferably, different promoters are used to confer expression of said genes or proteins.

Preferably, the promoter sequence used in the context of the present invention is operably linked to a coding sequence or open reading frame (ORF) encoding a LRR receptor-like kinase or a homolog, derivative and/or an immunologically active fragment thereof as defined supra.

“Dominant negative version or variant” refers to a mutant protein, which interferes with the activity of the corresponding wild-type protein.

“Downregulation of expression” as used herein means lowering levels of gene expression and/or levels of active gene product and/or levels of gene product activity. Decreases in expression may be accomplished by e.g. the addition of coding sequences or parts thereof in a sense orientation (if resulting in co-suppression) or in an antisense orientation relative to a promoter sequence and furthermore by e.g. insertion mutagenesis (e.g. T-DNA insertion or transposon insertion) or by gene silencing strategies as described by e.g. Angell and Baulcombe (1998—WO9836083), Lowe et al. (1989—WO9853083), Lederer et al. (1999—WO9915682) or Wang et al. (1999-WO9953050). Genetic constructs aimed at silencing gene expression may have the nucleotide sequence of said gene (or one or more parts thereof) contained therein in a sense and/or antisense orientation relative to the promoter sequence. Another method to downregulate gene expression comprises the use of ribozymes, e.g. as described in Atkins et al. 1994 (WO9400012), Lenee et al. 1995 (WO9503404), Lutziger et al. 2000 (WO0000619), Prinsen et al. 1997 (WO9713865) and Scott et al. 1997 (WO9738116). Modulating, including lowering, the level of active gene products or of gene product activity can be achieved by administering or exposing cells, tissues, organs or organisms to said gene product, a homolog, analogue, derivative and/or immunologically active fragment thereof. Immunomodulation is another example of a technique capable of downregulation levels of active gene product and/or of gene product activity and comprises administration of or exposing to or expressing antibodies to said gene product to or in cells, tissues, organs or organisms wherein levels of said gene product and/or gene product activity are to be modulated. Such antibodies comprise “plantibodies”, single chain antibodies, IgG antibodies and heavy chain camel antibodies as well as fragments thereof.

Modulating, including lowering, the level of active gene products or of gene product activity can futhermore be achieved by administering or exposing cells, tissues, organs or organisms to an inhibitor or activator of said gene product or the activity thereof. Such inhibitors or activators include proteins (comprising e.g. proteinases and kinases) and chemical compounds identified according to the current invention as described supra.

In the context of the invention the term “agonist” refers to a substance that can be either a protagonist or an antagonist, i.e. can have either positive or negative effects, can be an enhancer or an inhibitor or a modulator as well.

In the context of the current invention is envisaged the downregulation of the expression of a LRR receptor-like kinase gene as defined higher. The invention further comprises downregulation of levels of a LRR receptor-like kinase or of a LRR receptor-like kinase activity whereby said LRR receptor-like kinase has been defined supra.

By “cell fate and/or plant development and/or plant morphology and/or biochemistry and/or physiology” is meant that one or more developmental and/or morphological and/or biochemical and/or physiological characteristics of a plant is altered by the performance of one or more steps pertaining to the invention described herein.

“Cell fate” refers to the cell-type or cellular characteristics of a particular cell that are produced during plant development or a cellular process therefor, in particular during the cell cycle or as a consequence of a cell cycle process.

“Plant development” or the term “plant developmental characteristic” or similar term shall, when used herein, be taken to mean any cellular process of a plant that is involved in determining the developmental fate of a plant cell, in particular the specific tissue or organ type into which a progenitor cell will develop. Cellular processes relevant to plant development will be known to those skilled in the art. Such processes include, for example, morphogenesis, photomorphogenesis, shoot development, root development, vegetative development, reproductive development, stem elongation, flowering, and regulatory mechanisms involved in determining cell fate, in particular a process or regulatory process involving the cell cycle.

“Plant morphology” or the term “plant morphological characteristic” or similar term will, when used herein, be understood by those skilled in the art to refer to the external appearance of a plant, including any one or more structural features or combination of structural features thereof. Such structural features include the shape, size, number, position, colour, texture, arrangement, and patternation of any cell, tissue or organ or groups of cells, tissues or organs of a plant, including the root, stem, leaf, shoot, petiole, trichome, flower, petal, stigma, style, stamen, pollen, ovule, seed, embryo, endosperm, seed coat, aleurone, fibre, fruit, cambium, wood, heartwood, parenchyma, aerenchyma, sieve element, phloem or vascular tissue, amongst others.

“Plant biochemistry” or the term “plant biochemical characteristic” or similar term will, when used herein, be understood by those skilled in the art to refer to the metabolic and catalytic processes of a plant, including primary and secondary metabolism and the products thereof, including any small molecules, macromolecules or chemical compounds, such as but not limited to starches, sugars, proteins, peptides, enzymes, hormones, growth factors, nucleic acid molecules, celluloses, hemicelluloses, calloses, lectins, fibres, pigments such as anthocyanins, vitamins, minerals, micronutrients, or macronutrients, that are produced by plants.

“Plant physiology” or the term “plant physiological characteristic” or similar term will, when used herein, be understood to refer to the functional processes of a plant, including developmental processes such as growth, expansion and differentiation, sexual development, sexual reproduction, seed set, seed development, grain filling, asexual reproduction, cell division, dormancy, germination, light adaptation, photosynthesis, leaf expansion, fiber production, secondary growth or wood production, amongst others; responses of a plant to externally-applied factors such as metals, chemicals, hormones, growth factors, environment and environmental stress factors (eg. anoxia, hypoxia, high temperature, low temperature, dehydration, light, daylength, flooding, salt, heavy metals, amongst others), including adaptive responses of plants to said externally-applied factors.

Means for introducing recombinant DNA into plant tissue or cells include, but are not limited to, transformation using CaCl₂ and variations thereof, in particular the method described by Hanahan (1983), direct DNA uptake into protoplasts (Krens et al, 1982; Paszkowski et al, 1984), PEG-mediated uptake to protoplasts (Armstrong et al, 1990) microparticle bombardment, electroporation (Fromm et al., 1985), microinjection of DNA (Crossway et al., 1986), microparticle bombardment of tissue explants or cells (Christou et al, 1988), vacuum-infiltration of tissue with nucleic acid, or in the case of plants, T-DNA-mediated transfer from Agrobacterium to the plant tissue as described essentially by An et al.(1985), Dodds et al. (1985), Herrera-Estrella et al. (1983a, 1983b). Methods for transformation of monocotyledonous plants are well known in the art and include Agrobacterium-mediated transformation (Cheng et al. 1997—WO9748814; Hansen 1998—WO9854961, Hiei et al. 1994—WO9400977; Hiei et al. 1998—WO9817813; Rikiishi et al. 1999—WO9904618; Saito et al. 1995—WO9506722), microprojectile bombardment (Adams et al. 1999—U.S. Pat. No. 5,969,213; Bowen et al. 1998—U.S. Pat. No. 5,736,369; Chang et al. 1994 —WO9413822; Lundquist et al. 1999—U.S. Pat. No. 5,874,265/U.S. Pat. No. 5,990,390; Vasil and Vasil 1995 —U.S. Pat. No. 5,405,765; Walker et al. 1999—U.S. Pat. No. 5,955,362), DNA uptake (Eyal et al. 1993 —WO9318168), microinjection of Agrobacterium cells (von Holt 1994—DE4309203) and sonication (Finer et al. 1997—U.S. Pat. No. 5,693,512).

For microparticle bombardment of cells, a microparticle is propelled into a cell to produce a transformed cell. Any suitable ballistic cell transformation methodology and apparatus can be used in performing the present invention. Exemplary apparatus and procedures are disclosed by Stomp et al. (U.S. Pat. No. 5,122,466) and Sanford and Wolf (U.S. Pat. No. 4,945,050). When using ballistic transformation procedures, the gene construct may incorporate a plasmid capable of replicating in the cell to be transformed. Examples of microparticles suitable for use in such systems include 1 to 5 μm gold spheres. The DNA construct may be deposited on the microparticle by any suitable technique, such as by precipitation.

A whole plant may be regenerated from the transformed or transfected cell, in accordance with procedures well known in the art. Plant tissue capable of subsequent clonal propagation, whether by organogenesis or embryogenesis, may be transformed with a gene construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The term “organogenesis”, as used herein, means a process by which shoots and roots are developed sequentially from meristematic centers.

The term “embryogenesis”, as used herein, means a process by which shoots and roots develop together in a concerted fashion (not sequentially), whether from somatic cells or gametes.

Preferably, the plant is produced according to the inventive method is transfected or transformed with a genetic sequence, or amenable to the introduction of a protein, by any art-recognized means, such as microprojectile bombardment, microinjection, Agrobactetrium-mediated transformation (including the “flower dip” transformation method; Bechtold and Pelletier 1998, Trieu et al. 2000), protoplast fusion, or electroporation, amongst others. Most preferably said plant is produced by Agrobacterium-mediated transformation.

The “seedling” is the juvenile plant that arises from the mature embryo after seed germination.

With “differentiation of a cell” it is understood that the cell develops unique features to be engaged for a specific function. Mostly differentiation is irreversible.

Agrobacterium-mediated transformation or agrolistic transformation of plants, yeast, moulds or filamentous fungi is based on the transfer of part of the transformation vector sequences, called the T-DNA, to the nucleus and on integration of said T-DNA in the genome of said eukaryote.

With “Agrobacterium” is meant a member of the Agrobacteriaceae, more preferably Agrobacterium or Rhizobacterium and most preferably Agrobacterium tumefaciens.

With “T-DNA”, or transferred DNA, is meant that part of the transformation vector flanked by T-DNA borders which is, after activation of the Agrobacterium vir genes, nicked at the T-DNA borders and is transferred as a single stranded DNA to the nucleus of an eukaryotc cell.

When used herein, with “T-DNA borders”, “T-DNA border region”, or “border region” are meant either right T-DNA border (RB) or left T-DNA border (LB). Such a border comprises a core sequence flanked by a border inner region as part of the T-DNA flanking the border and/or a border outer region as part of the vector backbone flanking the border. The core sequences comprise 22 bp in case of octopine-type vectors and 25 bp in case of nopaline-type vectors. The core sequences in the right border region and left border region form imperfect repeats. Border core sequences are indispensable for recognition and processing by the Agrobacterium nicking complex consisting of at least VirD1 and VirD2. Core sequences flanking a T-DNA are sufficient to promote transfer of said T-DNA. However, efficiency of transformation using transformation vectors carrying said T-DNA solely flanked by said core sequences is low. Border inner and outer regions are known to modulate efficiency of T-DNA transfer (Wang et al. 1987). One element enhancing T-DNA transfer has been characterized and resides in the right border outer region and is called overdrive (Peralta et al. 1986, van Haaren et al. 1987).

With “T-DNA transformation vector” or “T-DNA vector” is meant any vector encompassing a T-DNA sequence flanked by a right and left T-DNA border consisting of at least the right and left border core sequences, respectively, and used for transformation of any eukaryotic cell.

With “T-DNA vector backbone sequence” or “T-DNA vector backbone sequences” is meant all DNA of a T-DNA containing vector that lies outside of the T-DNA borders and, more specifically, outside the nicking sites of the border core imperfect repeats.

The current invention includes optimized T-DNA vectors such that vector backbone integration in the genome of a eukaryotic cell is minimized or absent. With “optimized T-DNA vector” is meant a T-DNA vector designed either to decrease or abolish transfer of vector backbone sequences to the genome of a eukaryotic cell. Such T-DNA vectors are known to the one familiar with the art and include those described by Hanson et al. (1999) and by Stuiver et al. (1999—WO9901563).

The current invention clearly considers the inclusion of a DNA sequence comprising the promoter sequence of the present invention encoding a LRR receptor-like kinase, homolog, derivative or immunologically active fragment thereof as defined supra, in any T-DNA vector comprising binary transformation vectors, super-binary transformation vectors, co-integrate transformation vectors, Ri-derived transformation vectors as well as in T-DNA carrying vectors used in agrolistic transformation.

With “binary transformation vector” is meant a T-DNA transformation vector comprising: a T-DNA region comprising at least one gene of interest and/or at least one selectable marker active in the eukaryotic cell to be transformed; and a vector backbone region comprising at least origins of replication active in E. coli and Agrobacterium and markers for selection in E. coli and Agrobacterium.

The T-DNA borders of a binary transformation vector can be derived from octopine-type or nopaline-type Ti plasmids or from both. The T-DNA of a binary vector is only transferred to a eukaryotic cell in conjunction with a helper plasmid. Also known in the art are multiple binary vector Agrobacterium strains for efficient co-transformation of plants (Bidney and Scelonge 2000—WO0018939).

With “helper plasmid” is meant a plasmid that is stably maintained in Agrobacterium and is at least carrying the set of vir genes necessary for enabling transfer of the T-DNA. Said set of vir genes can be derived from either octopine-type or nopaline-type Ti plasmids or from both.

With “super-binary transformation vector” is meant a binary transformation vector additionally carrying in the vector backbone region a vir region of the Ti plasmid pTiBo542 of the super-virulent A. tumefaciens strain A281 (Hiei et al. 1994 EP0604662, Hiei et al. 1995—EP0687730). Super-binary transformation vectors are used in conjunction with a helper plasmid.

With “co-integrate transformation vector” is meant a T-DNA vector at least comprising: a T-DNA region comprising at least one gene of interest and/or at least one selectable marker active in plants; and a vector backbone region comprising at least origins of replication active in Escherichia coli and Agrobacterium, and markers for selection in E. coli and Agrobacterium, and a set of vir genes necessary for enabling transfer of the T-DNA.

The T-DNA borders and said set of vir genes of a said T-DNA vector can be derived from either octopine-type or nopaline-type Ti plasmids or from both.

With “Ri-derived plant transformation vector” is meant a binary transformation vector in which the T-DNA borders are derived from a Ti plasmid and said binary transformation vector being used in conjunction with a ‘helper’ Ri-plasmid carrying the necessary set of vir genes.

As used herein, the term “selectable marker gene” or “selectable marker” or “marker for selection” includes any gene which confers a phenotype on a cell in which it is expressed to facilitate the identification and/or selection of cells which are transfected or transformed with a gene construct of the invention or a derivative thereof. Suitable selectable marker genes contemplated herein include the ampicillin resistance (Amp′, tetracycline resistance gene (Tc′), bacterial kanamycin resistance gene (Kan′), phosphinothricin resistance gene, neomycin phosphotransferase gene (npfll), hygromycin resistance gene, β-glucuronidase (GUS) gene, chloramphenicol acetyltransferase (CAT) gene, green fluorescent protein (gfp) gene (Haseloff et al, 1997), and luciferase gene, amongst others.

With “agrolistics”, “agrolistic transformation” or “agrolistic transfer” is meant here a transformation method combining features of Agrobacterium-mediated transformation and of biolistic DNA delivery. As such, a T-DNA containing target plasmid is co-delivered with DNA/RNA enabling in plant a production of VirD1 and VirD2 with or without VirE2 (Hansen and Chilton 1996; Hansen et al. 1997; Hansen and Chilton 1997—WO9712046). The present invention further describes an approach to remove from transformed cells a stably integrated foreign DNA sequence by recombination involving a recombinase and recombination sites.

With “foreign DNA” is meant any DNA sequence that is introduced in the host's genome by recombinant techniques. Said foreign DNA includes e.g. a T-DNA sequence or a part thereof such as the T-DNA sequence comprising the selectable marker in an expressible format. Foreign DNA furthermore include intervening DNA sequences as defined supra.

With “recombination event” is meant either a site-specific recombination event or a recombination event effected by transposon ‘jumping’.

With “recombinase” is meant either a site-specific recombinase or a transposase.

With “recombination” site is meant either site-specific recombination sites or transposon border sequences.

With “site specific recombination event” is meant an event catalyzed by a system generally consisting of three elements: a pair of DNA sequences (the site-specific recombination sequences or sites) and a specific enzyme (the site-specific recombinase). The site-specific recombinase catalyzes a recombination reaction only between two site-specific recombination sequences depending on the orientation of the site-specific recombination sequences. Sequences intervening between two site-specific recombination sites will be inverted in the presence of the site-specific recombinase when the site-specific recombination sequences are oriented in opposite directions relative to one another (i.e. inverted repeats). If the site-specific recombination sequences are oriented in the same direction relative to one another (i.e. direct repeats), then any intervening sequences will be deleted upon interaction with the site-specific recombinase. Thus, if the site-specific recombination sequences are present as direct repeats at both ends of a foreign DNA sequence integrated into a eukaryotic genome, such integration of said sequences can subsequently be reversed by interaction of the site-specific recombination sequences with the corresponding site specific recombinase. A number of different site specific recombinase systems can be used including but not limited to the Cre/lox system of bacteriophage P1, the FLP/FRT system of yeast, the Gin recombinase of phage Mu, the Pin recombinase of E. coli, the PinB, PinD and PinF from Shigella, and the R/RS system of Zygosaccharomyces rouxil. Recombinases generally are integrases, resolvases or flippases. Also dual-specific recombinases can be used in conjunction with direct or indirect repeats of two different site-specific recombination sites corresponding to the dual-specific recombinase (Baszczynski et al. 1999 —WO9925840). The preferred site-specific recombinase systems are the bacteriophage P1 Cre/lox, the yeast FLP/FRT and the Z. rouxii R/RS systems. In these systems a recombinase (Cre, FLP or R) interact specifically with its respective site-specific recombination sequence (lox, FRT, or RS respectively) to invert or excise the intervening sequences. The site-specific recombination sequences for each of these two systems are relatively short (34 bp for 10× and 47 bp for FRT). Some of these systems have already been used with high efficiency in plants such as tobacco (Dale et al. 1990, Onouchi et al. 1991, Sugita et al. 2000) and Arabidopsis (Osbome et al. 1995, Onouchi et al. 1995). Site-specific recombination systems have many applications in plant molecular biology including methods for control of homologous recombination (e.g. Hodges et al. 1996—U.S. Pat. No. 5,527,695), for targeted insertion, gene stacking, etc. (Baszczynski et al. 1999—WO9925821) and for resolution of complex T-DNA integration patterns or for excision of a selectable marker (Ow et al. 1999—WO9923202).

Although the site-specific recombination sequences must be linked to the ends of the DNA to be excised or to be inverted, the gene encoding the site specific recombinase may be located elsewhere. For example, the recombinase gene could already be present in the eukaryote's DNA or could be supplied by a later introduced DNA fragment either introduced directly into cells, through crossing or through cross-pollination. Alternatively, a substantially purified recombinase protein could be introduced directly into the eukaryotic cell, e.g. by micro-injection or particle bombardment. Typically, the site-specific recombinase coding region will be operably linked to regulatory sequences enabling expression of the site-specific recombinase in the eukaryotic cell.

With “recombination event effected by transposon jumping” or “transposase-mediated recombination” is meant a recombination event catalyzed by a system consisting of three elements: a pair of DNA sequences (the transposon border sequences) and a specific enzyme (the transposase). The transposase catalyzes a recombination reaction only between two transposon border sequences which are arranged as inverted repeats. A number of different transposon/transposase systems can be used including but not limited to the Ds/Ac system, the Spm system and the Mu system. These systems originate from corn but it has been shown that at least the Ds/Ac and the Spm system also function in other plants (Fedoroff et al. 1993, Schlappi et al. 1993, Van Sluys et al. 1987). Preferred are the Ds- and the Spm-type transposons which are delineated by 11 bp- and 13 bp-border sequences, respectively.

Although the transposon border sequences must be linked to the ends of the DNA to be excised, the gene encoding the transposase may be located elsewhere. For example, the recombinase gene could already be present in the eukaryote's DNA or could be supplied by a later introduced DNA fragment either introduced directly into cells, through crossing or through cross-pollination. Alternatively, a substantially purified transposase protein could be introduced directly into cells, e.g. by microinjection or by particle bombardment.

As part of the current invention, transposon border sequences are included in a foreign DNA sequence such that they lie outside said DNA sequence and transform said DNA into a transposon-like entity that can move by the action of a transposase.

As transposons often reintegrate at another locus of the host's genome, segregation of the progeny of the hosts in which the transposase was allowed to act might be necessary to separate transformed hosts containing e.g. only the transposon footprint and transformed hosts still containing the foreign DNA.

In performing the present invention, the genetic element is preferably induced to mobilize, such as, for example, by the expression of a recombinase protein in the cell which contacts the integration site of the genetic element and facilitates a recombination event therein, excising the genetic element completely, or alternatively, leaving a “footprint”, generally of about 20 nucleotides in length or greater, at the original integration site. Those hosts and host parts that have been produced according to the inventive method can be identified by standard nucleic acid hybridization and/or amplification techniques to detect the presence of the mobilizable genetic element or a gene construct comprising the same. Alternatively, in the case of transformed host cells, tissues, and hosts wherein the mobilizable genetic element has been excised, it is possible to detect a footprint in the genome of the host which has been left following the excision event, using such techniques. As used herein, the term “footprint” shall be taken to refer to any derivative of a mobilizable genetic element or gene construct comprising the same as described herein which is produced by excision, deletion or other removal of the mobilizable genetic element from the genome of a cell transformed previously with said gene construct. A footprint generally comprises at least a single copy of the recombination loci or transposon used to promote excision. However, a footprint may comprise additional sequences derived from the gene construct, for example nucleotide sequences derived from the left border sequence, right border sequence, origin of replication, recombinase-encoding or transposase-encoding sequence if used, or other vector-derived nucleotide sequences. Accordingly, a footprint is identifiable according to the nucleotide sequence of the recombination locus or transposon of the gene construct used, such as, for example, a sequence of nucleotides corresponding or complementary to a lox site, frt site or RS site.

The term “cell cycle” means the cyclic biochemical and structural events associated with growth and with division of cells, and in particular with the regulation of the replication of DNA and mitosis. Cell cycle includes phases called: G0, Gap1 (G1), DNA synthesis (S), Gap2 (G2), and mitosis (M). Normally these four phases occur sequentially, however, the cell cycle also includes modified cycles wherein one or more phases are absent resulting in modified cell cycle such as endomitosis, acytokinesis, polyploidy, polyteny, and endoreduplication.

The term “cell cycle interacting protein”, “cell cycle protein” or “cell cycle control protein” as denoted herein means a protein which exerts control on or regulates or is required for the cell cycle or part thereof of a cell, tissue, organ or whole organism and/or DNA replication. It may also be capable of binding to, regulating or being regulated by cyclin dependent kinases or their subunits. The term also includes peptides, polypeptides, fragments, variant, homologs, alleles or precursors (eg preproproteins or preproteins) thereof.

Cell cycle control proteins and their role in regulating the cell cycle of eukaryotic organisms are reviewed in detail by John (1981) and the contributing papers therein (Norbury & Nurse 1992; Nurse 1990; Ormrod & Francis 1993) and the contributing papers therein (Doerner et al. 1996) which are incorporated by reference.(Elledge 1996; Francis, Francis & Halford 1995; Hirt et al. 1991; Mironov et al. 1999)

The term “cell cycle control genes” refers to any gene or mutant thereof which exerts control on or are required for chromosomal DNA synthesis and for mitosis (preprophase band, nuclear envelope, spindle formation, chromosome condensation, chromosome segregation, formation of new nuclei, formation of phragmoplast, duplication of microtubule-organizing center, etc) meiosis, cytokinesis, cell growth, endoreduplication, cell cycle control genes are also all genes exerting control on the above: homologs of CDKs, cyclins, E2Fs, Rb, CKI, Cks, and also any genes which interfere with the above, cyclin D, cdc25, Wee1, Nim1, MAP kinases, etc.

More specifically, cell cycle control genes are all genes involved in the control of entry and progression through S phase. They include, not exclusively, genes expressing “cell cycle control proteins” such as cyclin dependent kinases (CDK), cyclin dependent kinase inhibitors (CKI), D, E and A cyclins, E2F and DP transcription factors, pocket proteins, CDC7/DBF4 kinase, CDC6, MCM2-7, Orc proteins, cdc45, components of SCF ubiquitin ligase, PCNA, DNA-polymerase.

The term “cell cycle control protein” include cyclins A, B, C, D and E including CYCA1;1, CYCA2;1, CYCA3;1, CYCB1;1, CYCB1;2, CYC B2;2, CYCD1;1, CYCD2;1, CYCD3;1, and CYCD4;1 (Evans et al. 1983; Francis et al. 1998; Labbe et al. 1989; Murray & Kirschner 1989; Renaudin et al. 1996; Soni et al. 1995; Sorrell et al. 1999; Swenson, Farrell, & Ruderman 1986) cyclin dependent kinase inhibitor (CKI) proteins such as ICK1 (Wang, Fowke, & Crosby 1997), FL39, FL66, FL67 (PCT/EP98/05895), Sic1, Far1, Rum1, p21, p27, p57, p16, p15, pl8, p19 (Elledge 1996; Pines 1995), p14 and p14ARF; p13suc1 or CKS1At (De Veyider et al. 1997; Hayles & Nurse 1986) and nim-1 (Fantes 1989; Russell & Nurse 1986; Russell & Nurse 1987b; Russell & Nurse 1987a; Russell & Nurse 1986) homologues of Cdc2 such as Cdc2MsB (Hirt et al. 1993) CdcMs kinase (Bogre et al. 1997) cdc2 T14Y15 phosphatases such as Cdc25 protein phosphatase or p80cdc25 (Bell et al. 1993; Elledge 1996; Kumagai & Dunphy 1991; Russell et al. 1986) and Pyp3 (Elledge 1996) cdc2 protein kinase or p34cdc2 (Colasanti, Tyers, & Sundaresan 1991; Feiler & Jacobs 1990; Hirt et al. 1991; John, Sek, & Lee 1989; Lee & Nurse 1987; Nurse & Bissett 1981; Ormrod et al. 1993) cdc2a protein kinase (Hemerly et al. 1993) cdc2 T14Y15 kinases such as wee1 or p107wee1 (Elledge 1996; Russell et al. 1986; Russell et al. 1987b; Russell et al. 1987a; Sun et al. 1999) mik1 (Lundgren et al. 1991) and myt1 (Elledge 1996); cdc2 T161 kinases such as Cak and Civ (Elledge 1996); cdc2 T161 phosphatases such as Kap1 (Elledge 1996); cdc28 protein kinase or p34cdc28 (Nasmyth 1993; Reed, Hadwiger, & Lorincz 1985) p40MOl5 (Fesquet et al. 1993; Poon et al. 1993) chk1 kinase (Zeng et al. 1998) cds1 kinase (Zeng et al. 1998) growth-associated H1 kinase (Labbe et al. 1989; Lake & Salzman 1972; Langan 1978; Zeng et al. 1998) MAP kinases described by (Binarova et al. 1998; Bogre et al. 1999; Calderini et al. 1998; Wilson et al. 1999).

Other cell cycle control proteins that are involved in cyclin D-mediated entry of cells into G1 from G0 include pRb (Huntley et al. 1998; Xie et al. 1996) E2F, RIP, MCM7 and potentially the pRb-like proteins p107 and p130.

Other cell cycle control proteins that are involved in the formation of a pre-replicative complex at one or more origins of replication, such as, but not limited to, ORC, CDC6, CDC14, RPA and MCM proteins or in the regulation of formation of this pre-replicative complex, such as, but not limited to, the CDC7, DBF4 and MBF proteins.

For the present purpose, the term “cell cycle control protein” shall further be taken to include any one or more of those proteins that are involved in the turnover of any other cell cycle control protein, or in regulating the half-life of said other cell cycle control protein. The term “protein turnover” is to include all biochemical modifications of a protein leading to the physical or functional removal of said protein. Although not limited to these, examples of such modifications are phosphorylation, ubiquitination and proteolysis. Particularly preferred proteins which are involved in the proteolysis of one or more of any other of the above-mentioned cell cycle control proteins include the yeast-derived and animal-derived proteins, Skp1, Skp2, Rub1, Cdc20, cullins, CDC23, CDC27, CDC16, and plant-derived homologues thereof (Cohen-Fix & Koshland 1997; Hochstrasser 1998; Krek 1998; Lisztwan et al. 1998) and Plesse et al in (Francis et al. 1998)).

For the present, purpose, the term “cell cycle control genes” shall further be taken to include any one or more of those gene that are involved in the transcriptional regulation of cell cycle control gene expression such as transcription factors and upstream signal proteins. Additional cell cycle control genes are not excluded.

For the present purpose, the term “cell cycle control genes” shall further be taken to include any cell cycle control gene or mutant thereof, which is affected by environmental signals such as for instance stress, nutrients, pathogens, or by intrinsic signals such as the animal mitogens or the plant hormones (auxins, cytokinins, ethylene, gibberellic acid, abscisic acid and brassinosteroids).

The term “cell cycle progression”refers to the process of passing through the different cell cycle phases. The term “cell cycle progression rate” accordingly refers to the speed at which said cell cycle phases are run through or the time spans required to complete said cell cycle phases.

With “pathogen” is meant those organisms that have a negative effect on the physiological state of the plant or a part thereof. Some pathogens are for instanc viruses, bacteria, fungi, and parasitic plants, with plant “pests” is meant the group of nematodes as well as insects, which are able to attack and to damage the plant. “Plant cell” comprises any cell derived from any plant and existing in culture as a single cell, a group of cells or a callus. A plant cell may also be any cell in a developing or mature plant in culture or growing in nature.

“Plants” comprises all plants, including monocotyledonous and dicotyledonous plants.

With “yeast two-hybrid assay” is meant an assay that is based on the observation that many eukaryotic transcription factors comprise two domains, a DNA-binding domain (DB) and an activation domain (AD) which, when physically separated (i.e. disruption of the covalent linkage) do not effectuate target gene expression. Two proteins able to interact physically with one of said proteins fused to DB and the other of said proteins fused to AD will re-unite the DB and AD domains of the transcription factor resulting in target gen expression. The target gene in the yeast two-hybrid assay is usually a reporter gene such as the β-galactosidase gene. Interaction between protein partners in the yeast two-hybrid assay can thus be quantified by measuring the activity of the reporter gene product (Bartel and Fields 1997). Alternatively, a mammalian two-hybrid system can be used which includes e.g. a chimeric green fluorescent protein encoding reporter gene (Shioda et al. 2000). Yet another alternative consists of a bacterial two-hybrid system using e.g. HIS as reporter gene (Joung et al. 2000).

The term “fragment of a sequence” or “part of a sequence” means a truncated sequence of the original sequence referred to. The truncated sequence (nucleic acid or protein sequence) can vary widely in length; the minimum size being a sequence of sufficient size to provide a sequence with at least a comparable function and/or activity or the original sequence referred to, while the maximum size is not critical. In some applications, the maximum size usually is not substantially greater than that required to provide the desired activity and/or function(s) of the original sequence. Typically, the truncated amino acid or nucleotide sequence will range from about 5 to about 60 amino acids in length. More typically, however, the sequence will be a maximum of about 50 amino acids in lenght, preferably a maximum of about 60 amino acids. It is usually desirable to select sequences of at least about 10, 12 or 15 amino acids or nucleotides, up to a maximum of about 20 or 25 amino acids or nucleotides.

Furthermore, folding simulations and computer redesign of structural motifs of the protein of the invention can be performed using appropriate computer programs (Olszewski et al. 1996, Hoffman et al. 1995). Computer modeling of protein folding can be used for the conformational and energetic analysis of detailed peptide and protein models (Monge et al. 1995, Renouf et al. 1995). In particular, the appropriate programs can be used for the identification of interactive sites of the LRR receptor-like kinase of the present invention by computer assistant searches for complementary peptide sequences (Fassina and Melli 1994). Further appropriate computer systems for the design of protein and peptides are described in the prior art, for example in Berry and Brenner (1994), Wodak (1987), Pabo and Suchanek (1986). The results obtained form the above-described computer analysis can be used for, e.g. the preparation of peptidomimetics of the protein of the invention or fragments thereof. Such pseudopeptide analogues of the natural amino acid sequence of the protein may very efficiently mimic the parent protein (Benkirane et al. 1996). For example, incorporation of easily available achiral ω-amino acid residues into a protein of the invention or a fragment thereof results in the substitution of amino bonds by polymethylene units of an aliphatic chain, thereby providing a convenient strategy for constructing a peptidomimetic (Banerjee et al. 1996). Superactive peptidomimetic analogues of small peptide hormones in other systems are described in the prior art (Zhang et al. 1996). Appropriate peptidomimetics of the protein of the present invention can also be identified by the synthesis of peptidomimetic combinatorial libraries through successive amine alkylation and testing the resulting compounds, e.g., for their binding, kinase inhibitory and/or immunlogical properties. Methods for the generation and use of peptidomimetic combinatioral libraries are described in the prior art, for example in Ostresh et al. (1996) and Dorner et al. (1996).

Furthermore, a three-dimensional and/or crystallographic structure of the protein of the invention can be used for the design of peptidomimetic inhibitors of the biological activity of the protein of the invention (Rose et al. 1996, Ruterber et al. 1996).

To align sequences we used the Clustal V method described in Higgins and Sharp (Higgins D G & Sharp P M, Comput Appl Biosci. 1989 April; 5(2)151-3). The Clustal Method groups sequences into clusters by examining sequence distances between all pairs. Clusters are aligned as pairs then collectively as sequence groups to produce the overall alignment. After the multiple alignment is completed, a neighbor-joining method is employed to reconstruct phylogeny for the putative alignment.

1. Residue Weight Table

The Clustal method uses weight tables to construct multiple alignments. Residue weight tables are used in scoring protein and nucleotide alignments so that slightly mismatched residues, such as lie vs. Val amino acids or G vs. R nucleotides, are not scored the same as total mismatches.

If your sequences are protein, or a mixture of protein and DNA, choose between PAM250, PAM100, Structural and Identity.

1. iPAM250 is recommended for the Clustal method and is ideal for longer sequences or highly diverged sequences.

2. PAM100 is also used with the Clustal method and is most accurate for shorter sequences or sequences having little divergence.

3. iStructural is recommended for the Jotun Hein method and gives higher scores to residues that are chemically and spatially similar.

4. Identity may be used with Clustal or Jotun Hein methods, but is recommended only for the Clustal method. The table scores non-identities as zero, and identities as a default value of 10.

2. Method Parameters

A. Pairwise Alignment Parameters may include K-tuple, Gap Penalty, Window and Diagonals Saved:

1. Specify the K-tuple, expressed as the unit's length in nucleotides. Any stretch of K or more adjacent nucleotides that form an exact match in both sequences is a K-tuple. A lower K-tuple will find smaller matching regions; a higher value will find fewer, but better, matching regions of similarity.

2. Type in the Gap Penalty, the amount deducted from the score for each gap in the alignment. Gaps of different sizes carry the same penalty.

3. Specify the Window, the range the program searches for all possible or alternate K-tuples. An extremely high value may slow an alignment by considering subalignments far from the main diagonal.

4. Type in the number of Diagonals Saved. In the pairwise calculation, the best scoring diagonals for each pair in the matrix are saved and used in the clustering and scoring of ancestors.

B. Multiple Alignment Parameters include Gap Penalty and Gap Length Penalty.

1. Specify the Gap Penalty, the amount deducted from the score for each gap in the alignment. Gaps of different sizes carry the same penalty.

2. Specify the Gap Length Penalty, the value which will be deducted from the alignment score after first multiplying it by the number of gaps. Longer gaps have a greater penalty than shorter gaps.

Whenever in the current description reference is made to a “phylogenetic tree”, this phylogenetic tree is used to view phylogenetic relationships compatible with multiple sequence alignments. The length of each pair of branches represents the distance between sequence pairs, while the units at the bottom of the tree indicate the number of substitution events.

The phylogenetic tree gives a first estimate of the relationships between homologous sequences. It is highly recommended that your data be evaluated using additional methods before final conclusions are made about evolutionary relationships.

You can switch the view between Balanced Branches (phenogram) or Unbalanced Branches (cladogram). A phenogram averages the distances between ancestors in the tree. Dotted lines indicate a negative branch length, which may be a byproduct of averaging. In the cladogram, branch distances correspond to sequence divergence.

The compounds to be obtained or identified in the methods of the invention can be compounds that are able to bind to any of the nucleic acids, peptides or proteins of the invention. Other interesting compounds to be identified are compounds that modulate the expression of the genes or the proteins of the invention in such a way that either the expression of said gene or protein is enhanced or decreased by the action of said compound. Alternatively the compound can exert his action by directly or indirectly enhancing or decreasing the activity of any of the proteins of the invention.

Said compound or plurality of compounds may be comprised in, for example, samples, e.g., cell extracts from, e.g., plants, animals or microorganisms. Furthermore, said compound(s) may be known in the art but hitherto not known to be capable of suppressing or activating cell cycle interacting proteins. The reaction mixture may be a cell free extract of may comprise a cell or tissue culture. Suitable set ups for the method of the invention are known to the person skilled in the art and are, for example, generally described in Alberts et al., Molecular Biology of the Cell, third edition (1994), in particular Chapter 17. The plurality of compounds may be, e.g., added to the reaction mixture, culture medium or injected into the cell.

If a sample containing a compound or a plurality of compounds is identified in the method of the invention, then it is either possible to isolate the compound form the original sample identified as containing the compound capable of acting as an agonist, or one can further subdivide the original sample, for example, if it consists of a plurality of different compounds, so as to reduce the number of different substances per sample and repeat the method with the subdivisions of the original sample. Depending on the complexity of the samples, the steps described above can be performed several times, preferably until the sample identified according to the method of the invention only comprises a limited number of or only one substance(s). Preferably said sample comprises substances or similar chemical and/or physical properties, and most preferably said substances are identical. Preferably, the compound identified according to the above described method or its derivative is further formulated in a form suitable for the application in plant breeding or plant cell and tissue culture.

The invention, now being generally described, will be more readily understood by reference to the following examples, which are included merely for purposes of illustration of certain aspects and embodiments of the present invention and are not intended to limit the invention.

### DESCRIPTION OF FIGURES

FIG. 1. Models for stem-cell maintenance in the root and shoot meristem.

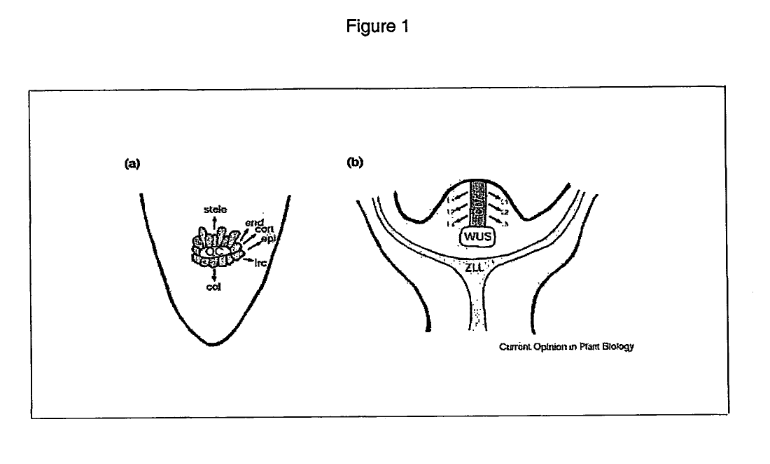

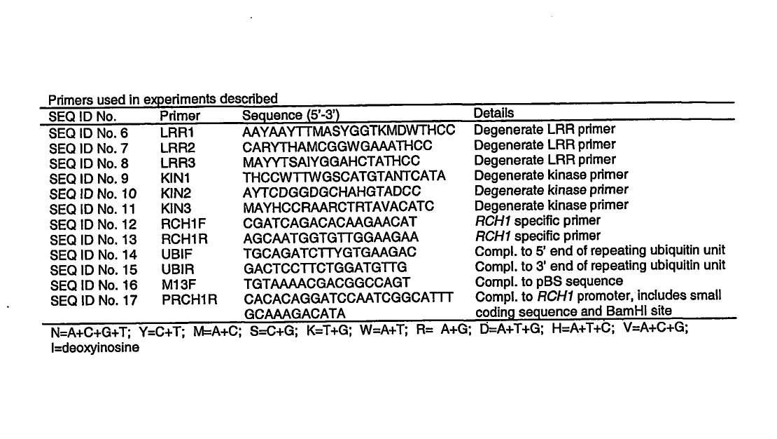

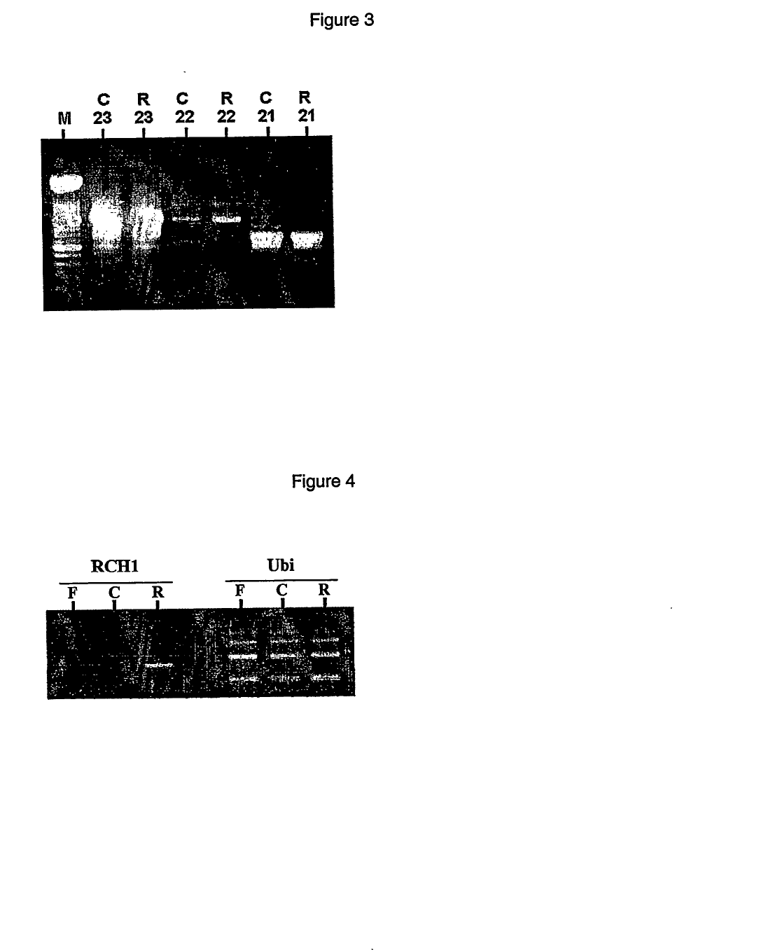

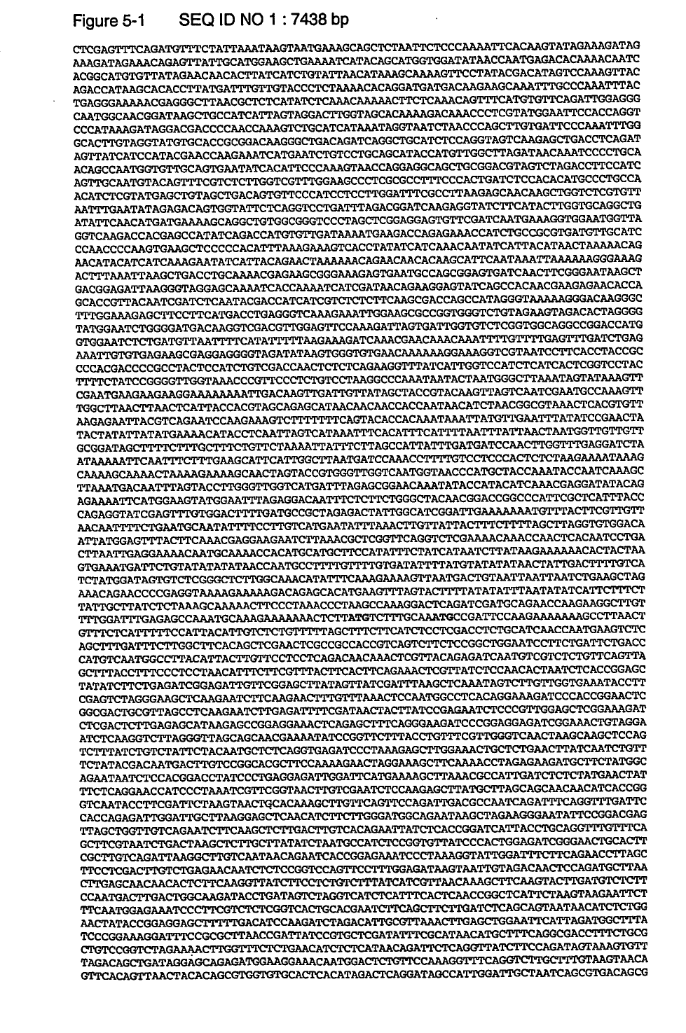

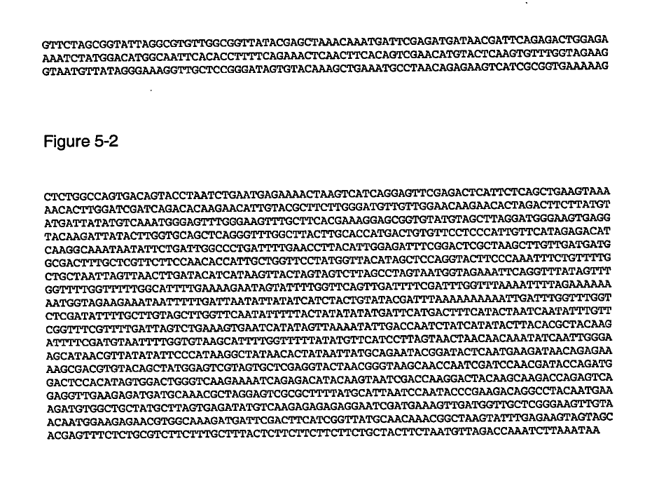

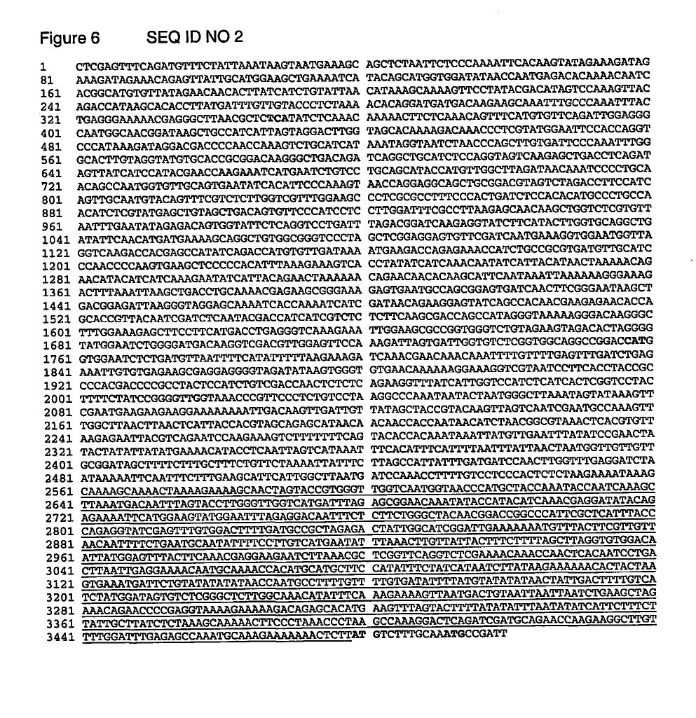

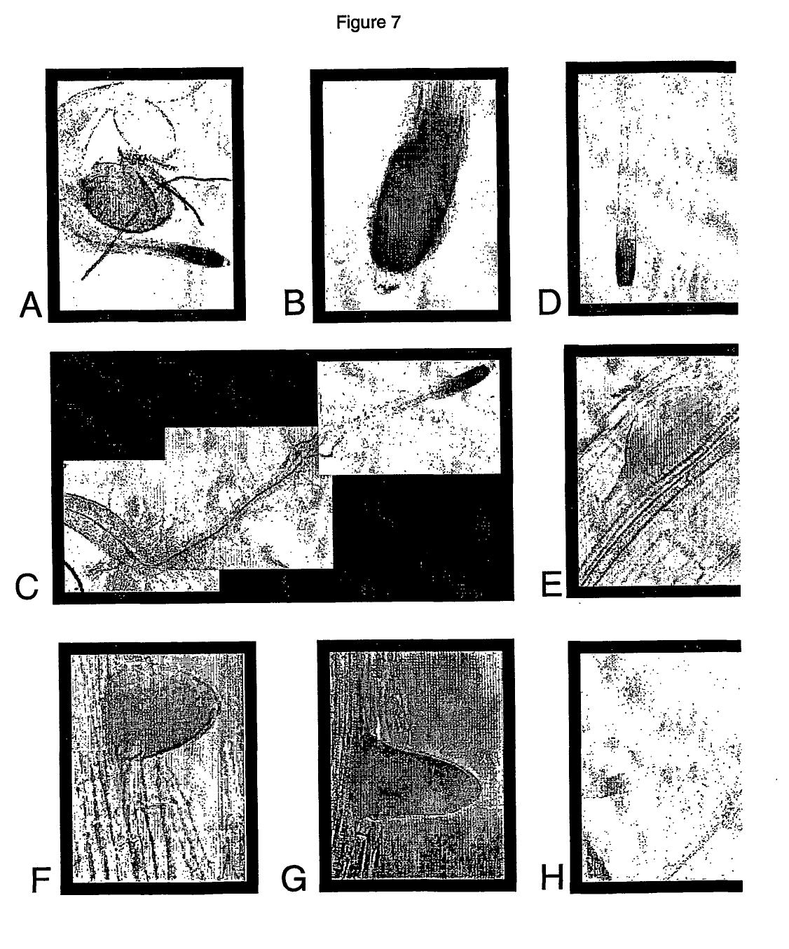

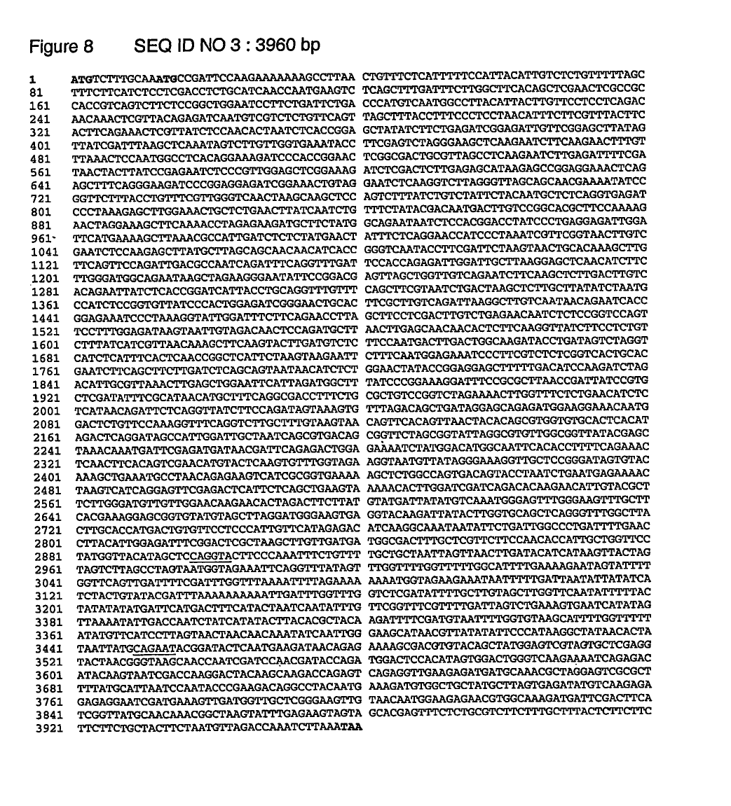

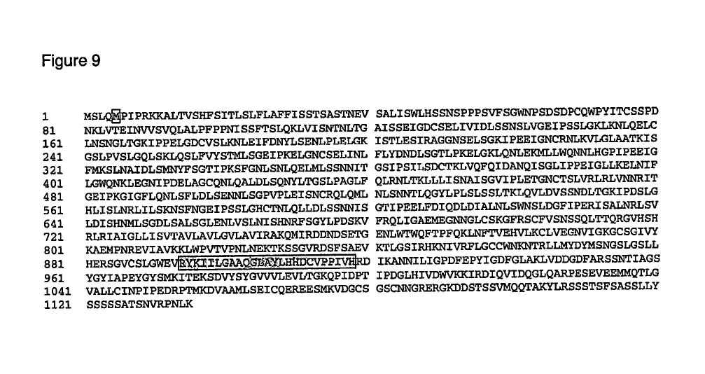

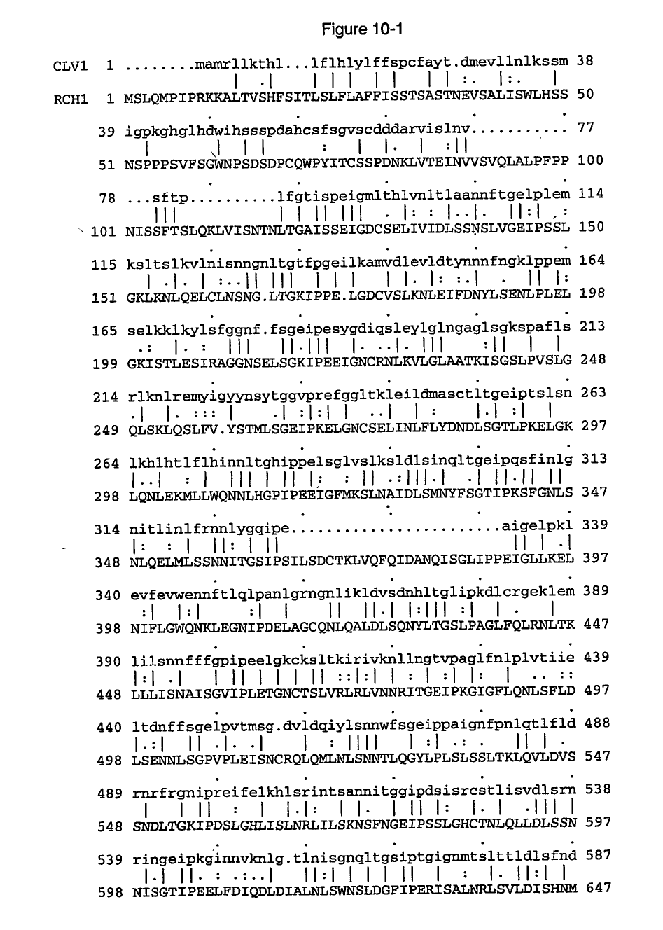

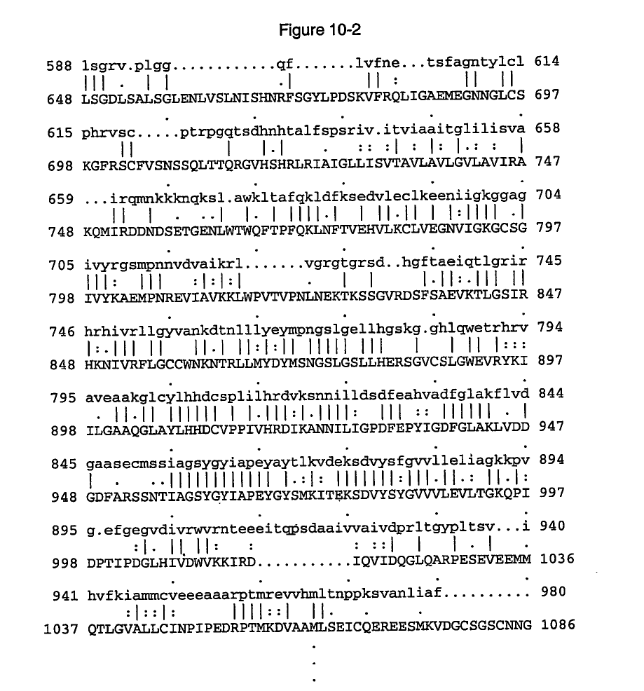

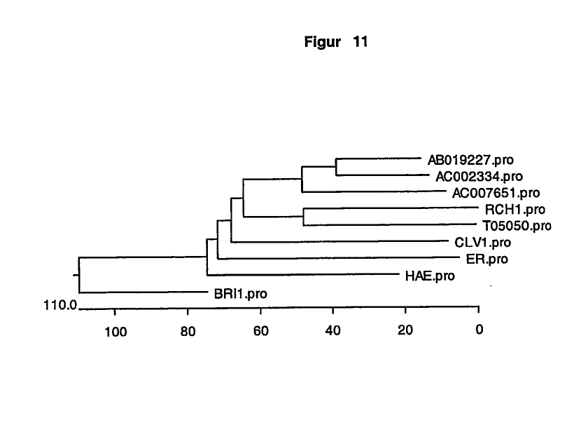

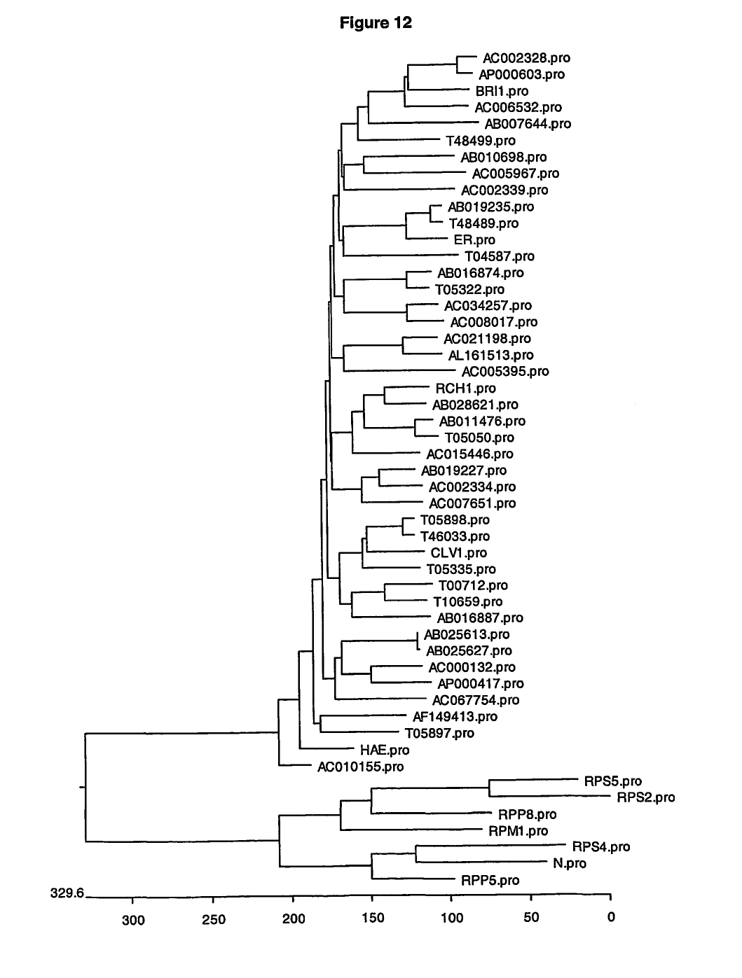

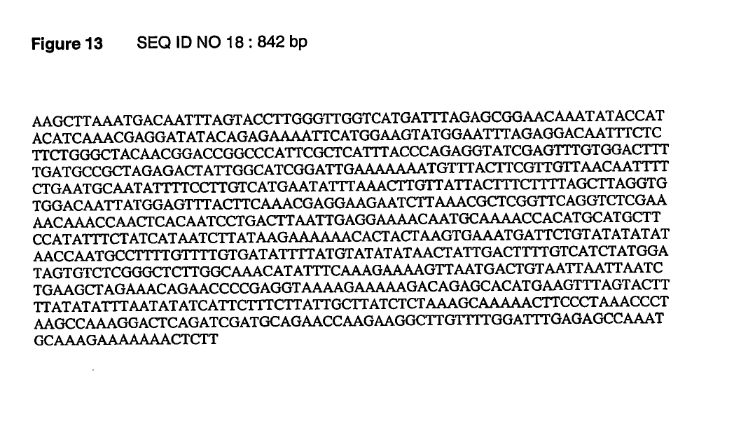

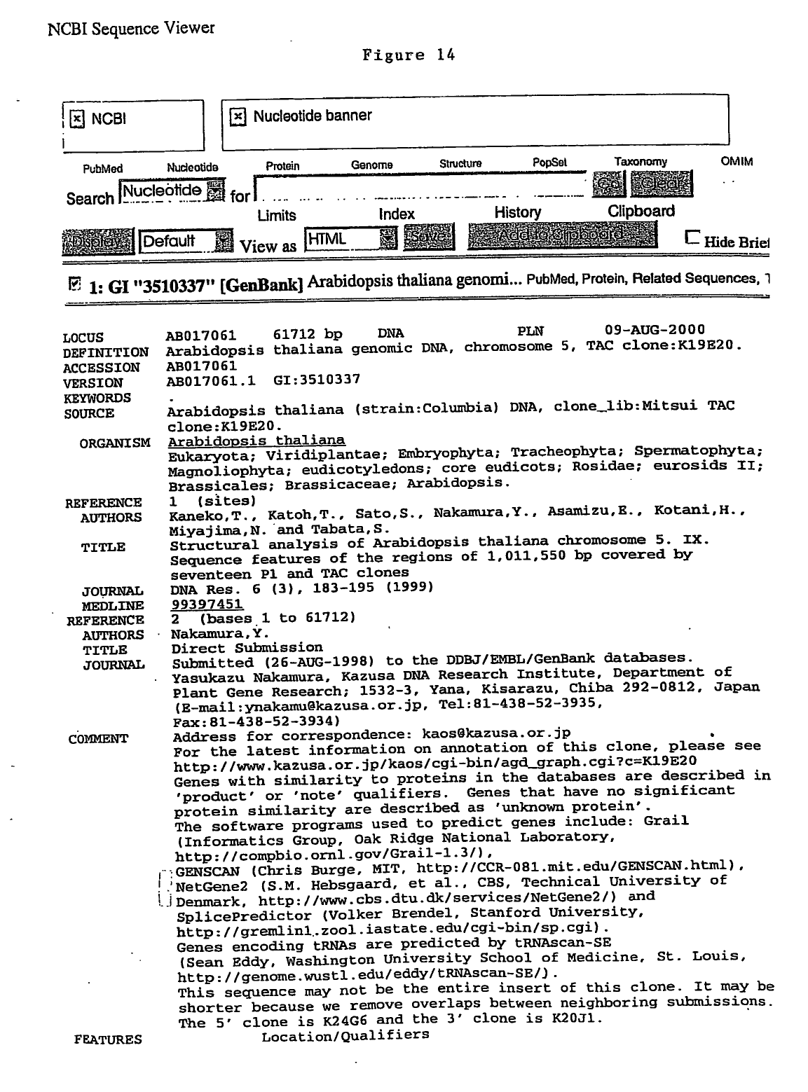

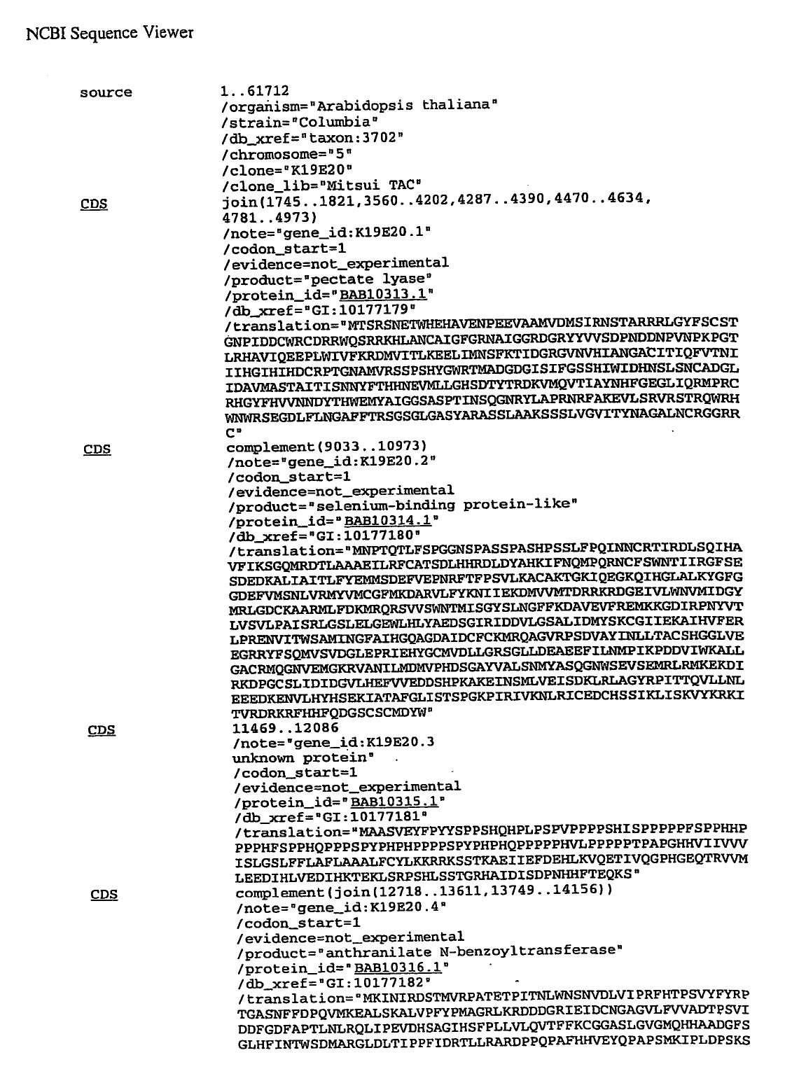

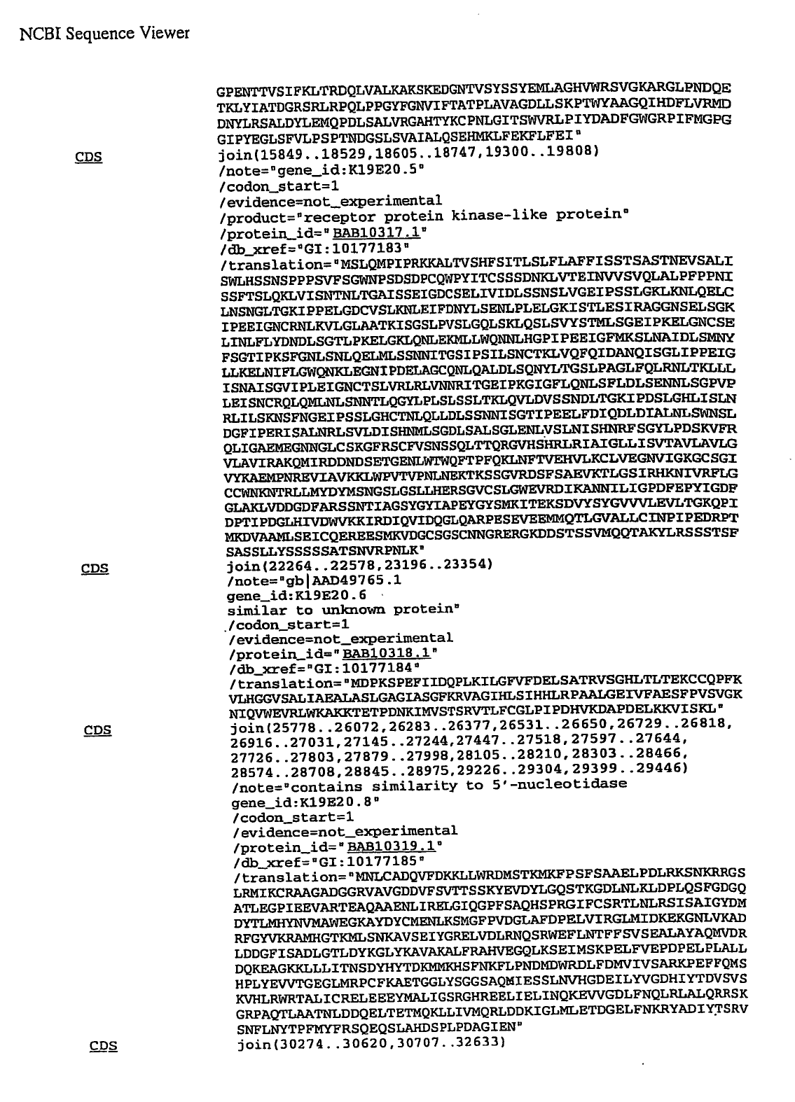

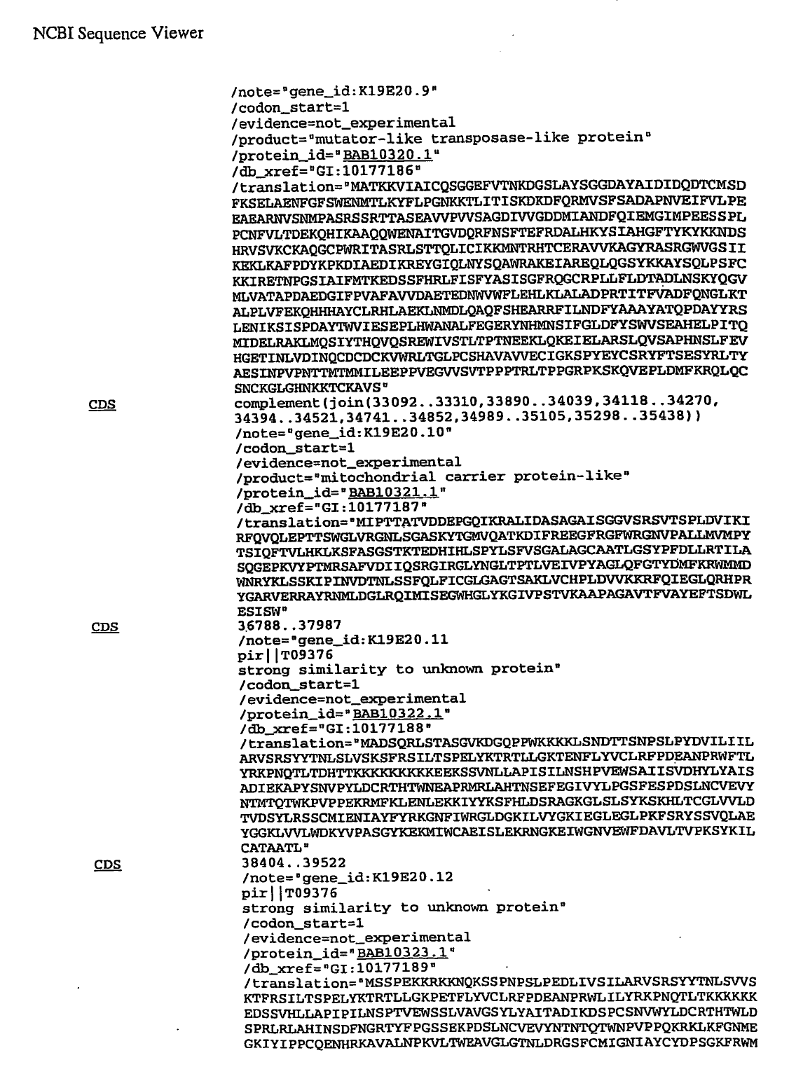

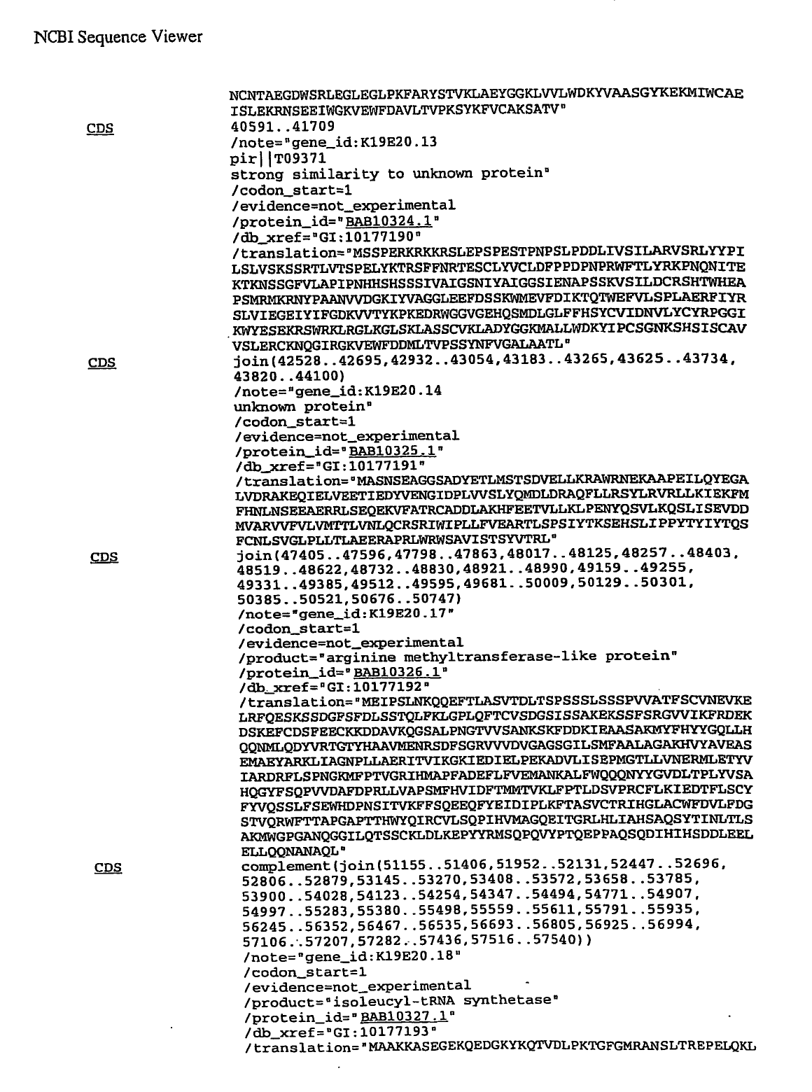

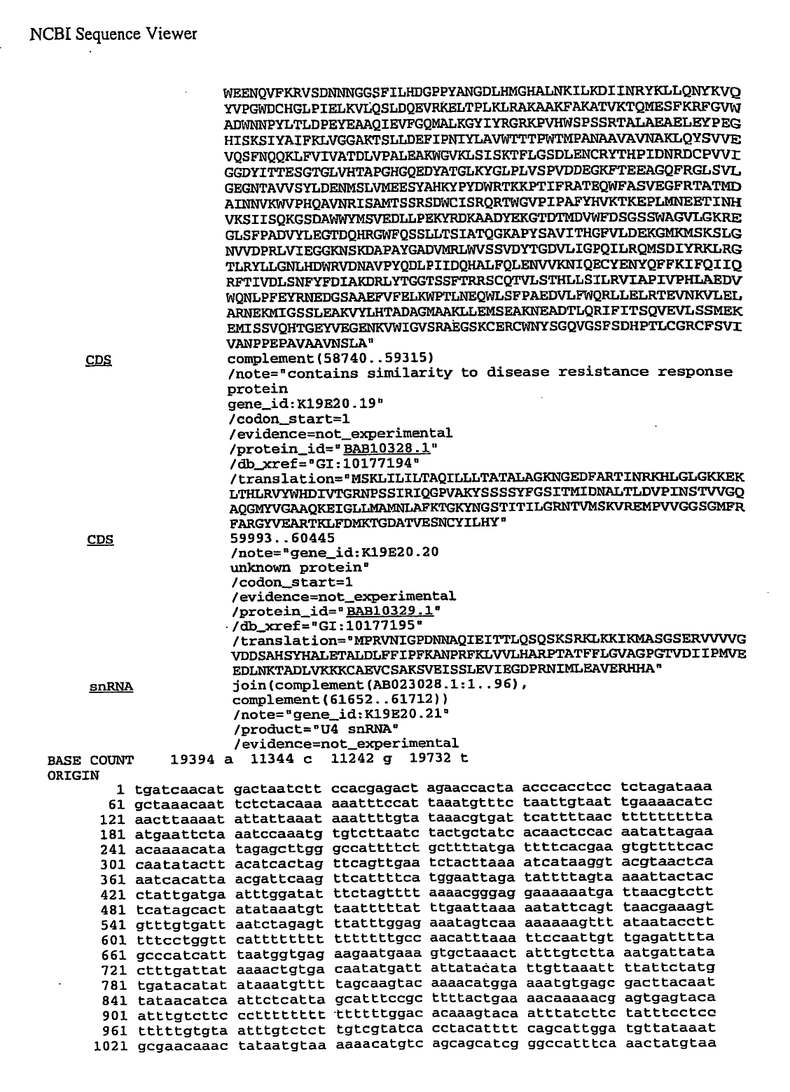

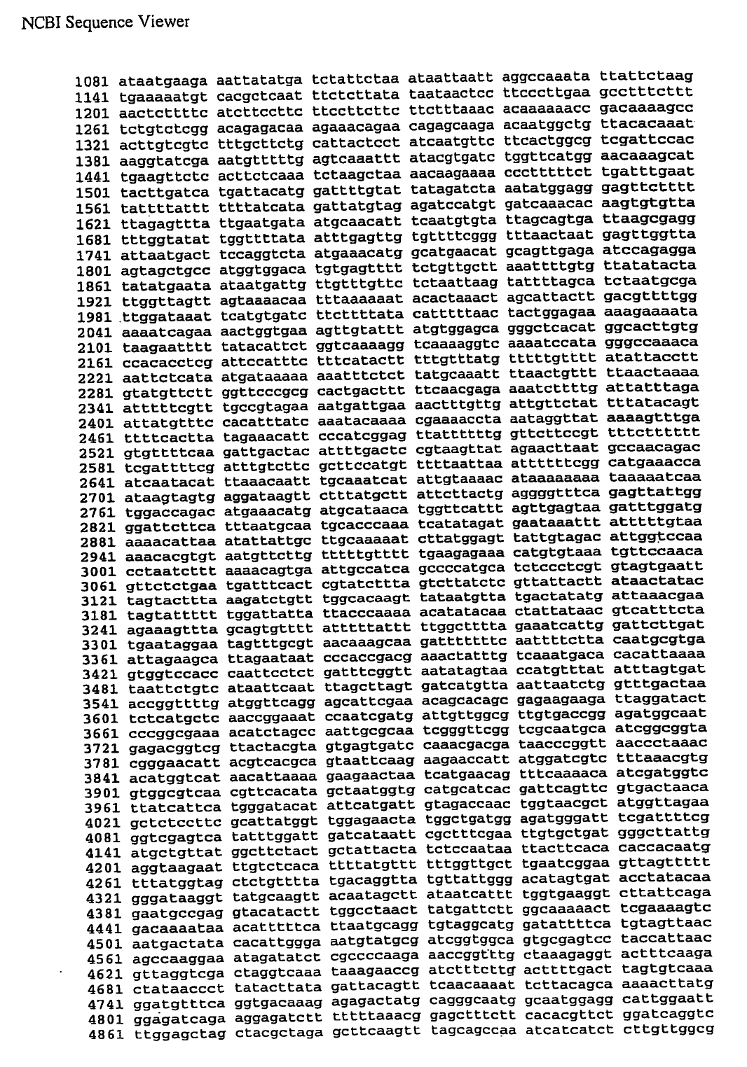

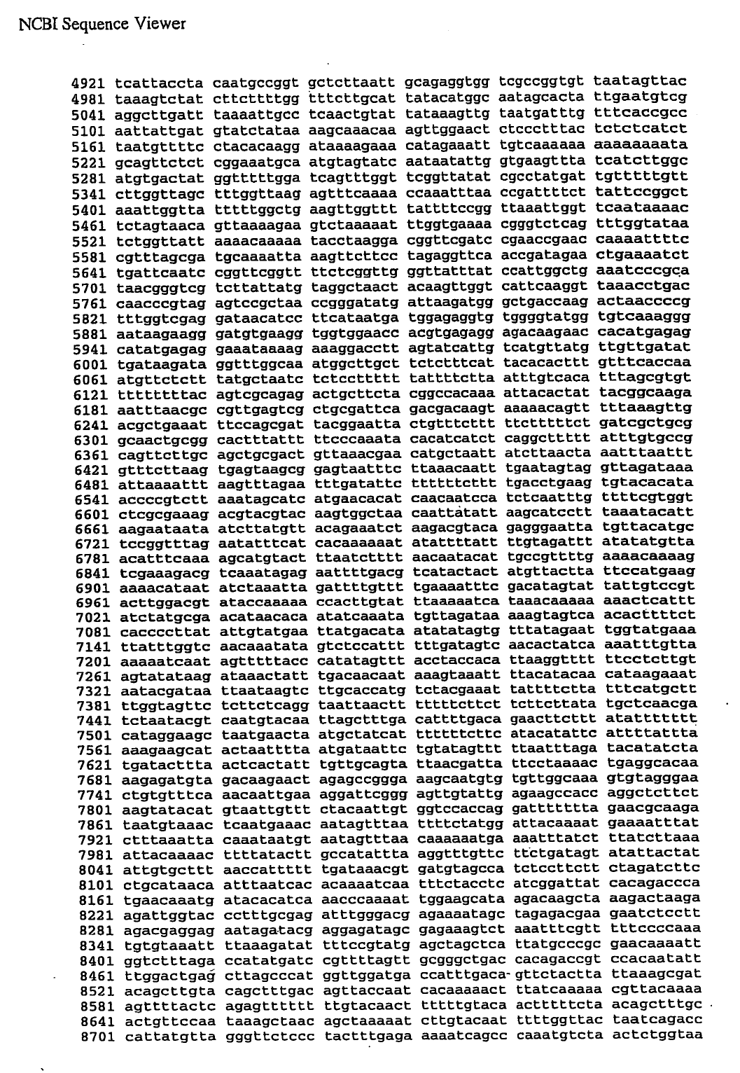

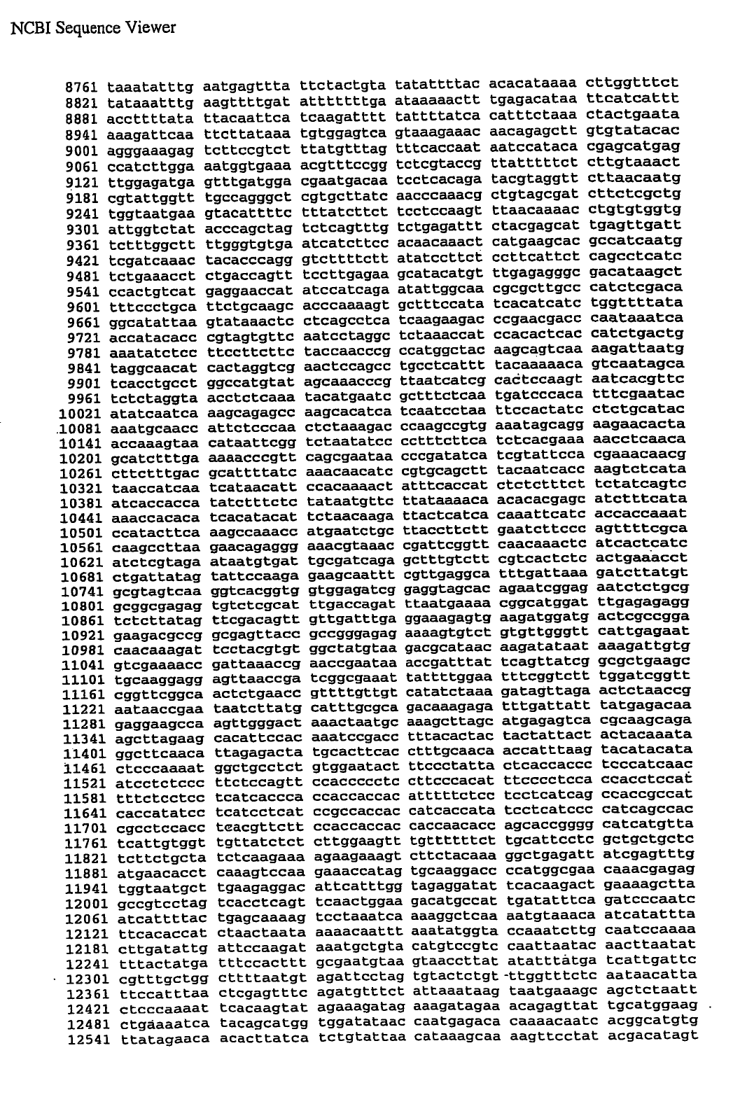

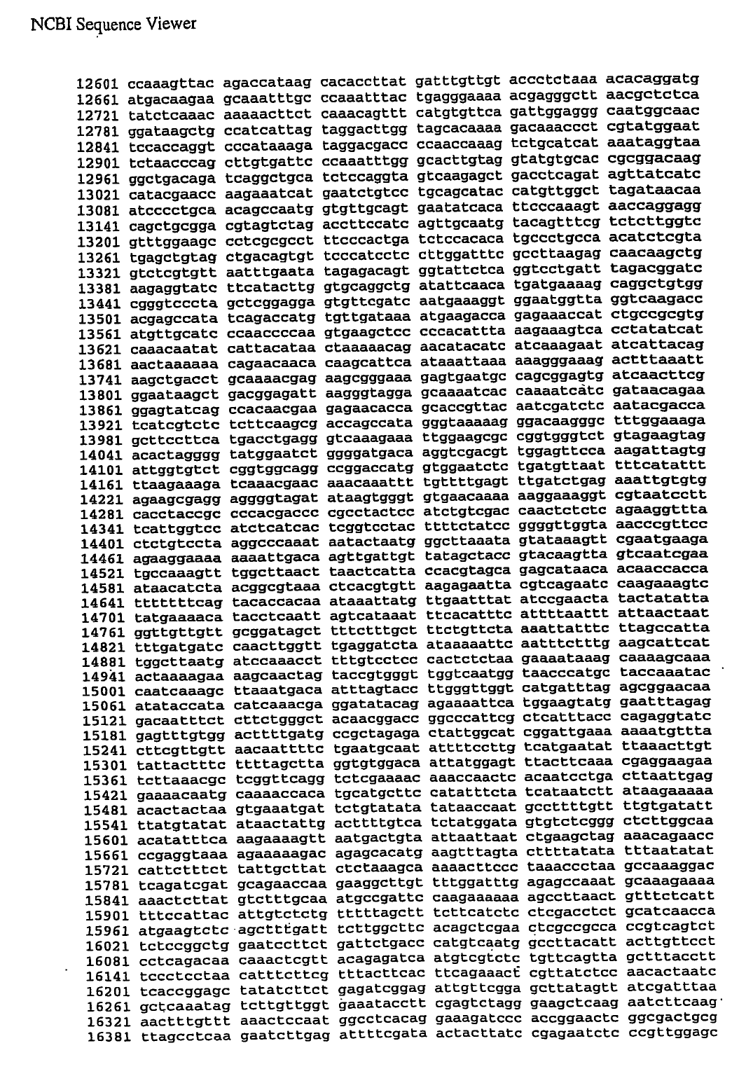

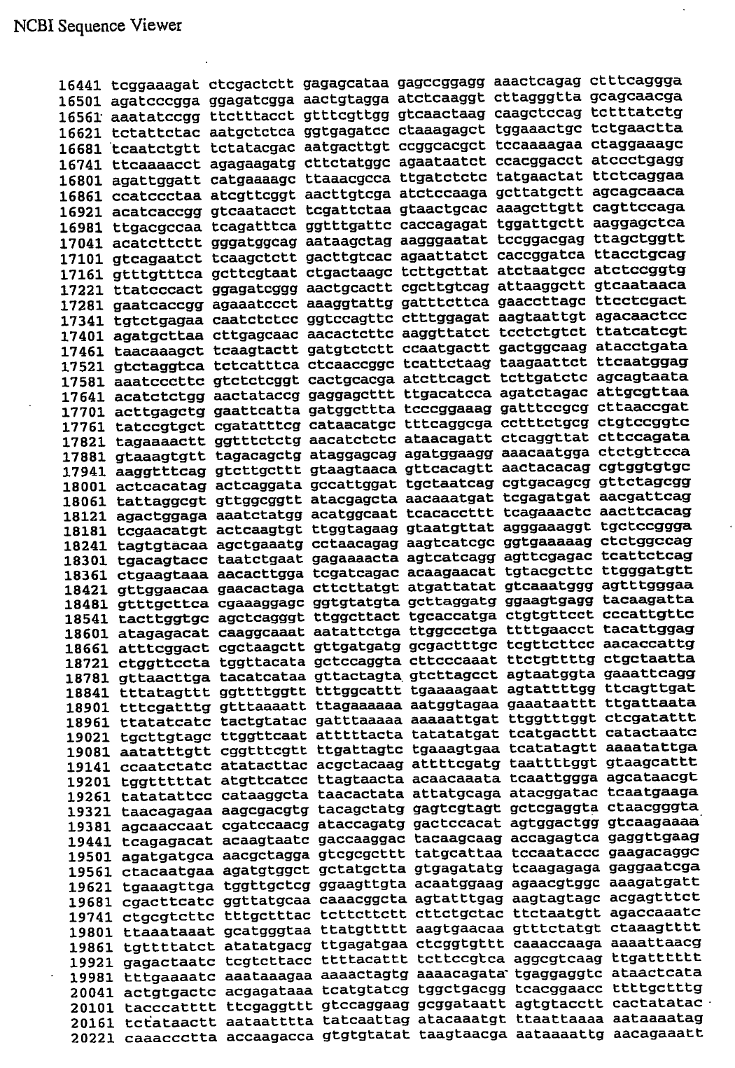

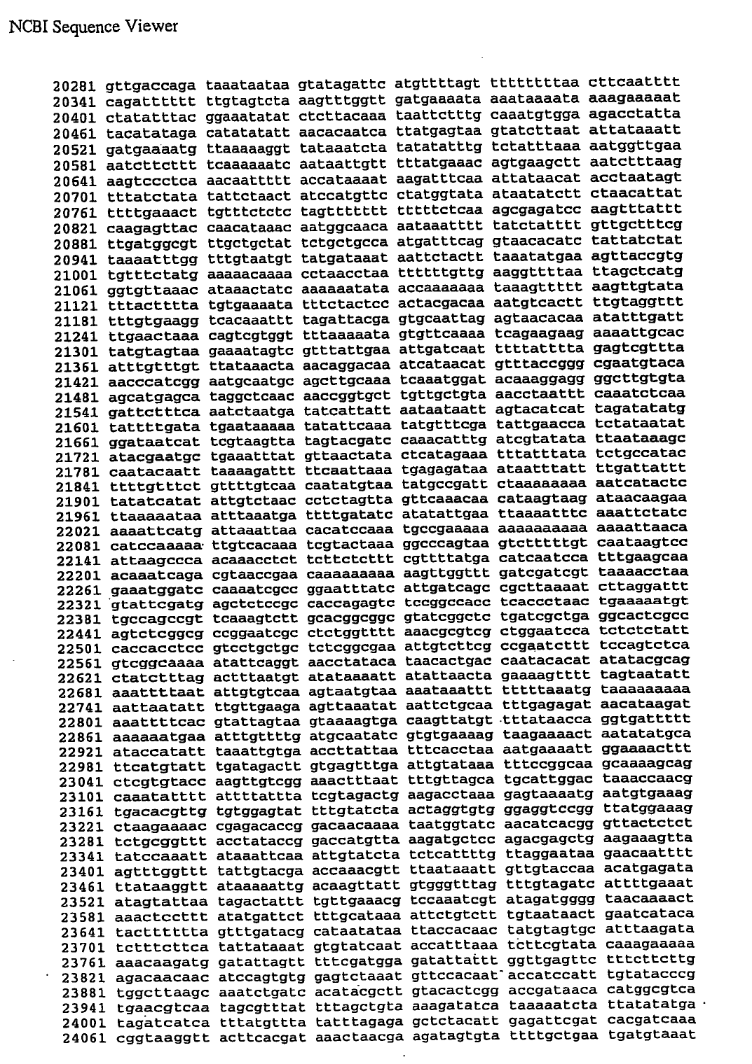

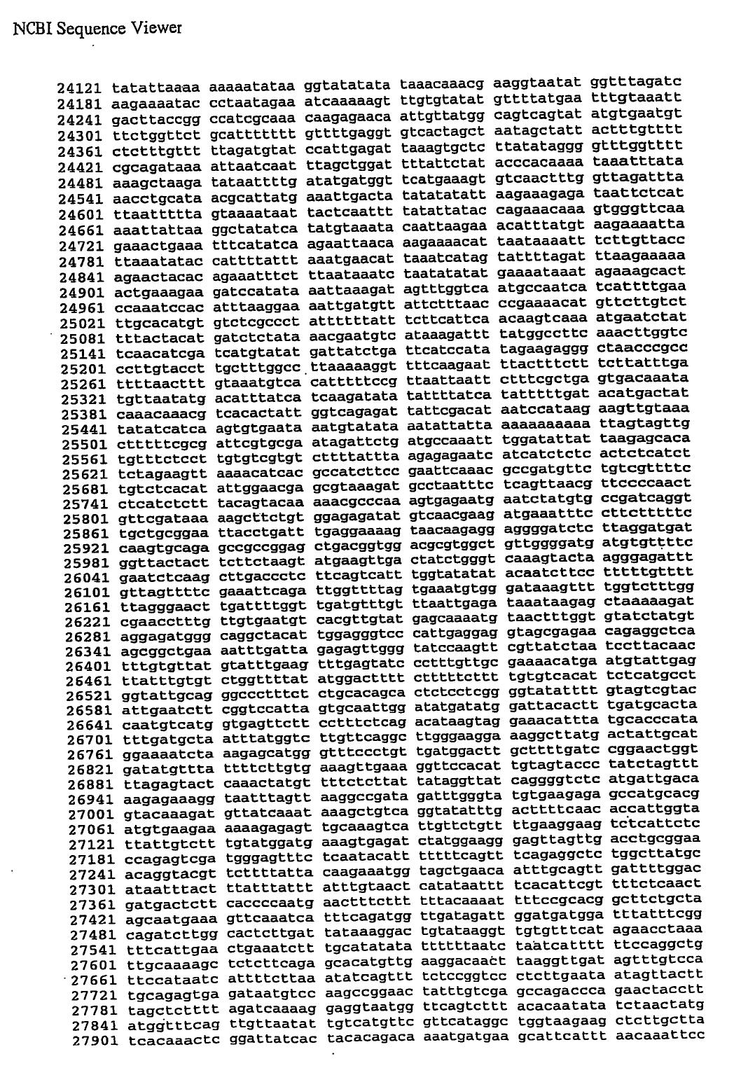

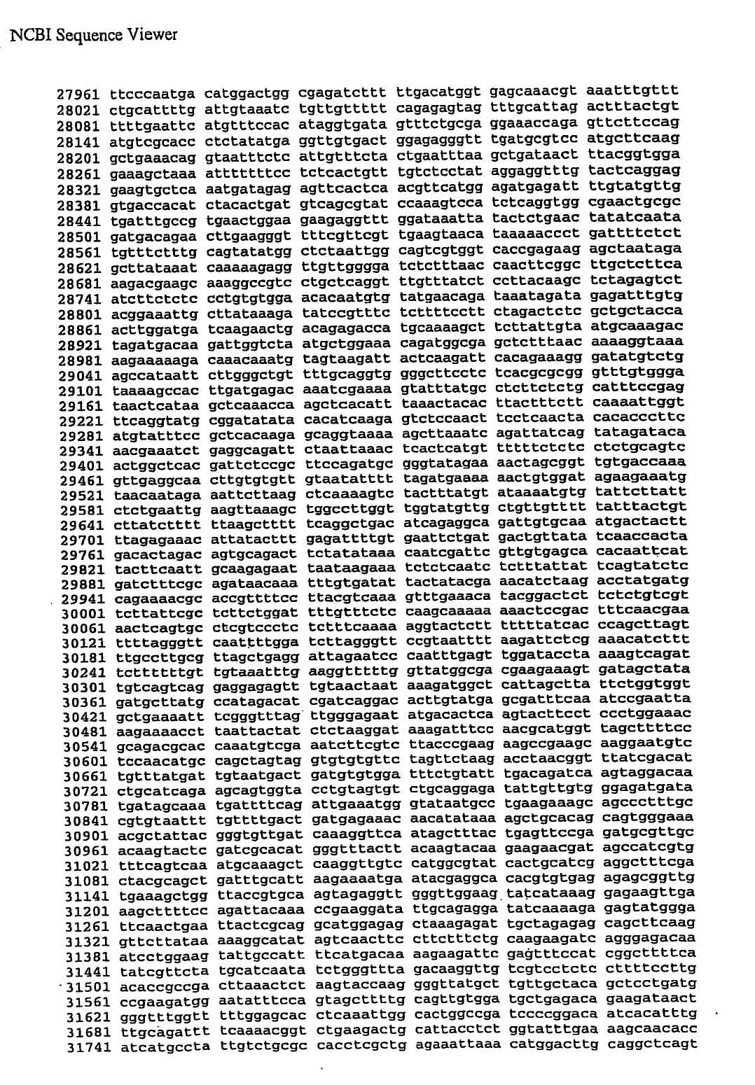

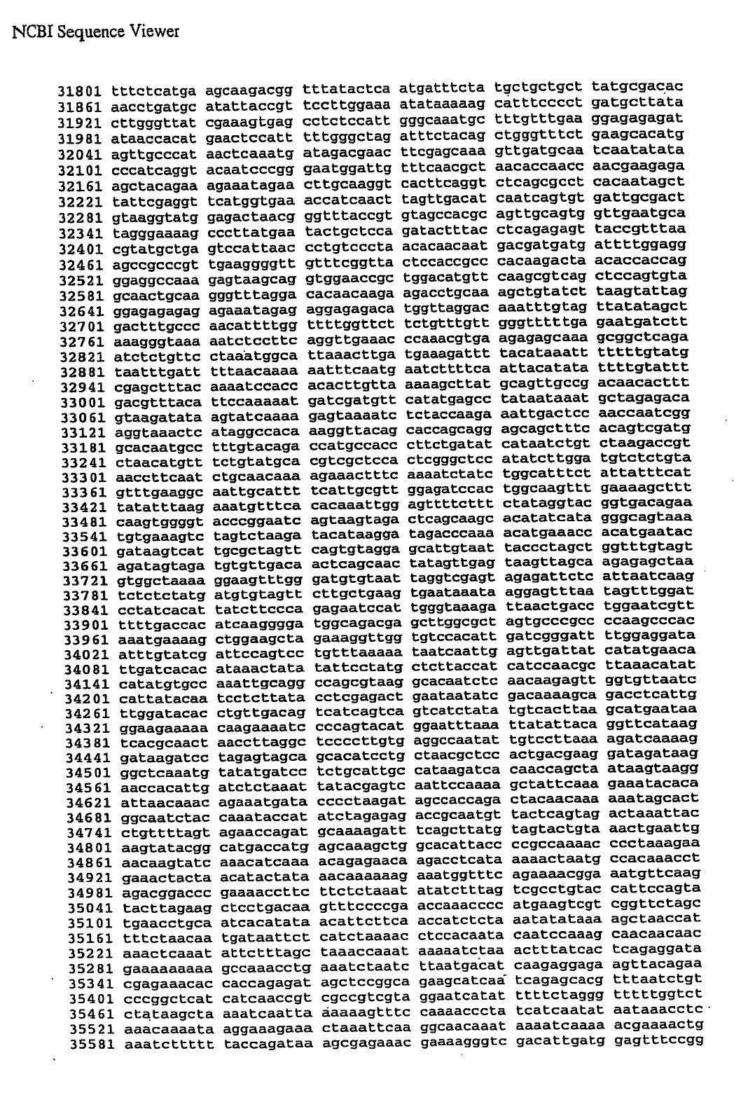

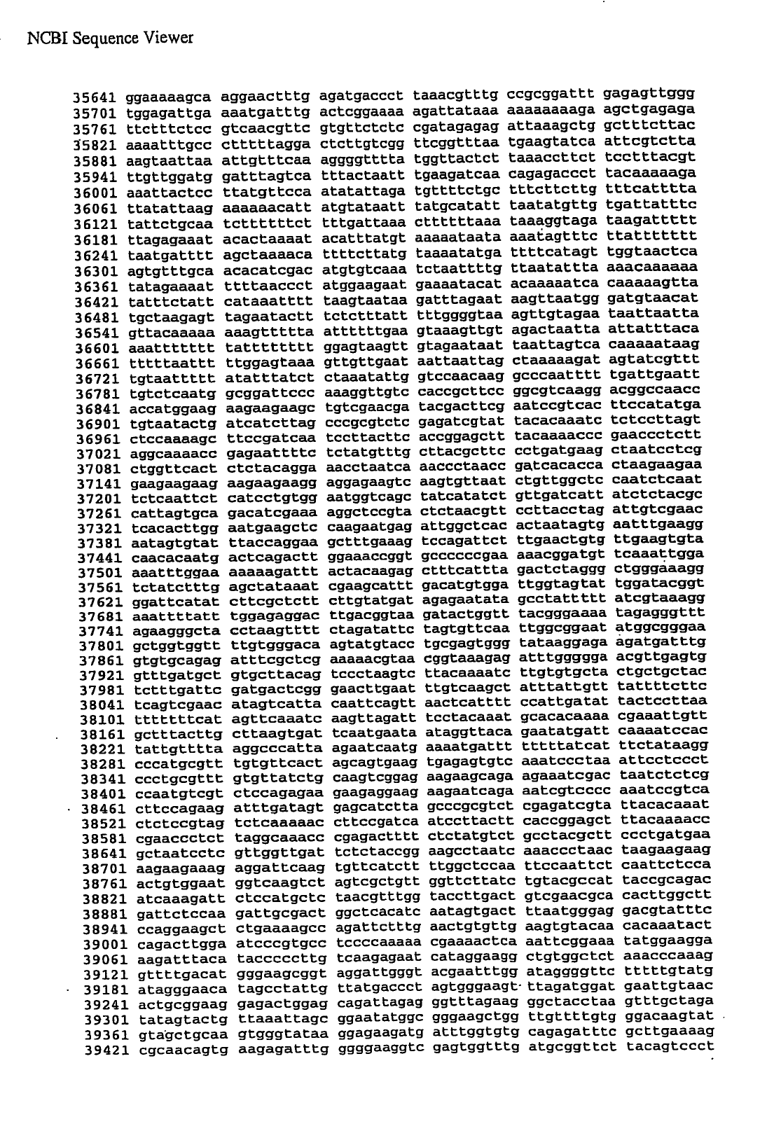

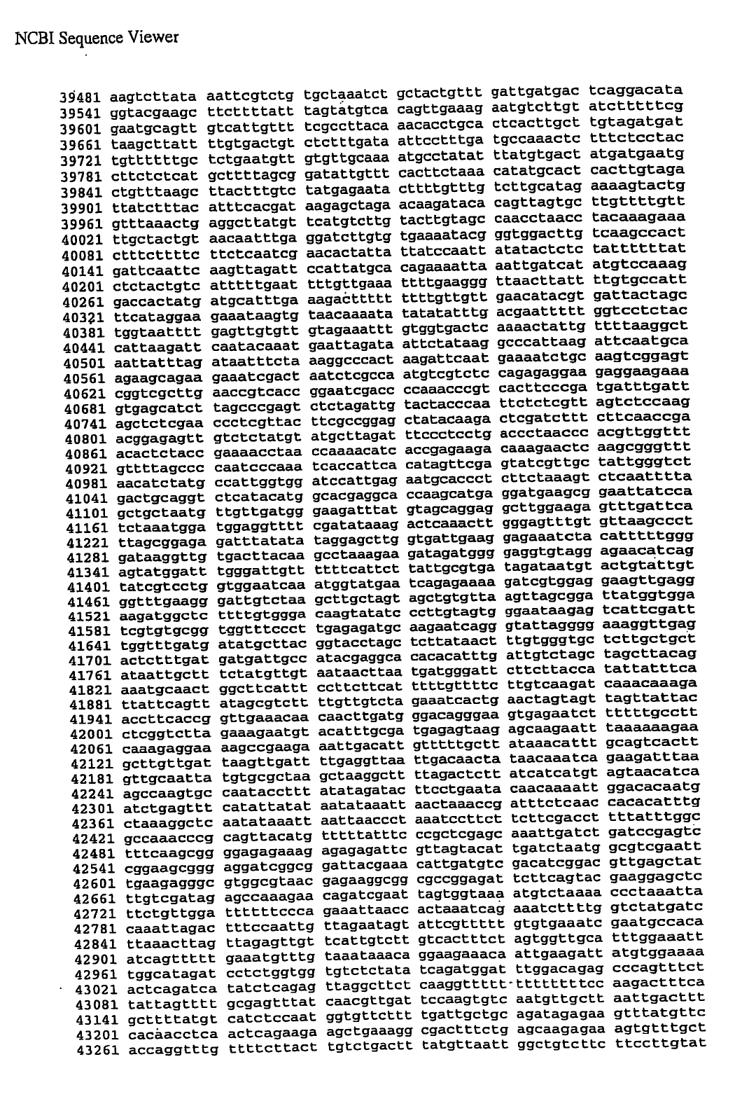

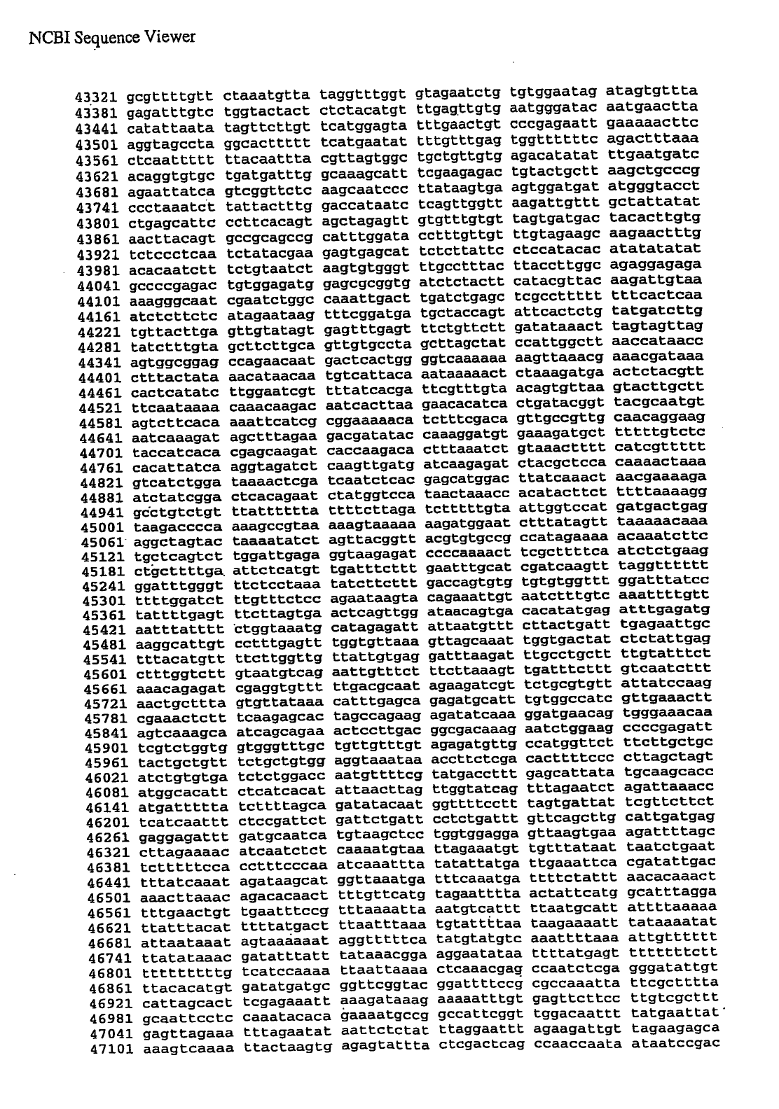

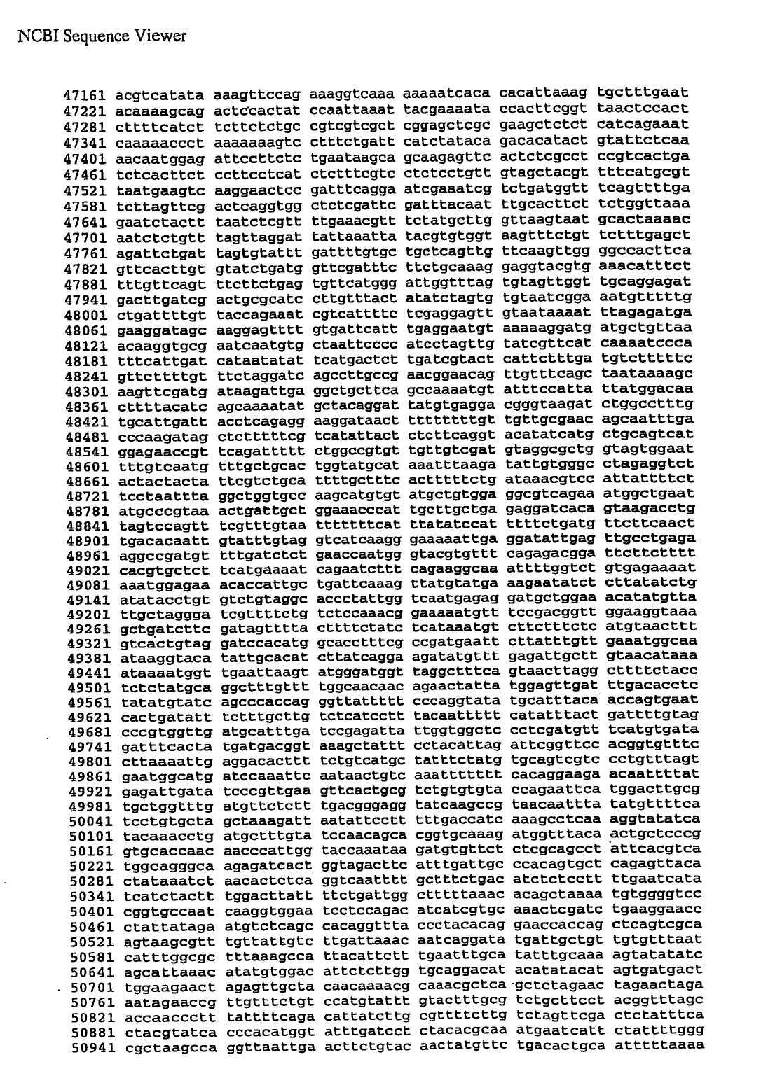

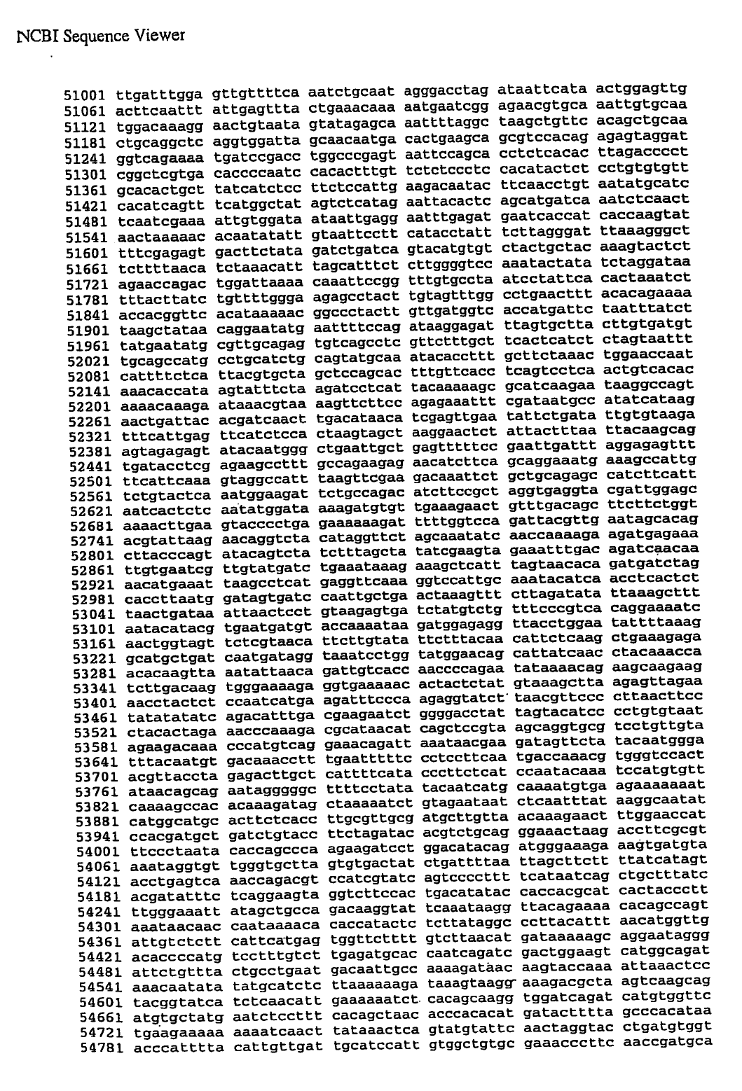

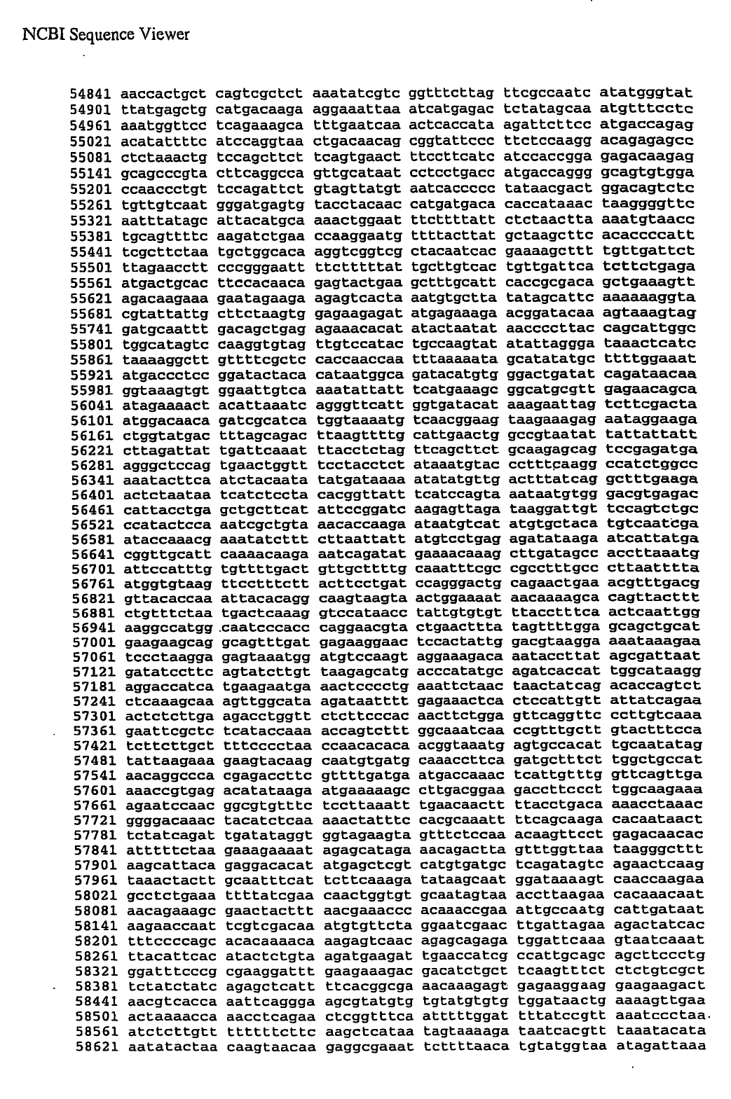

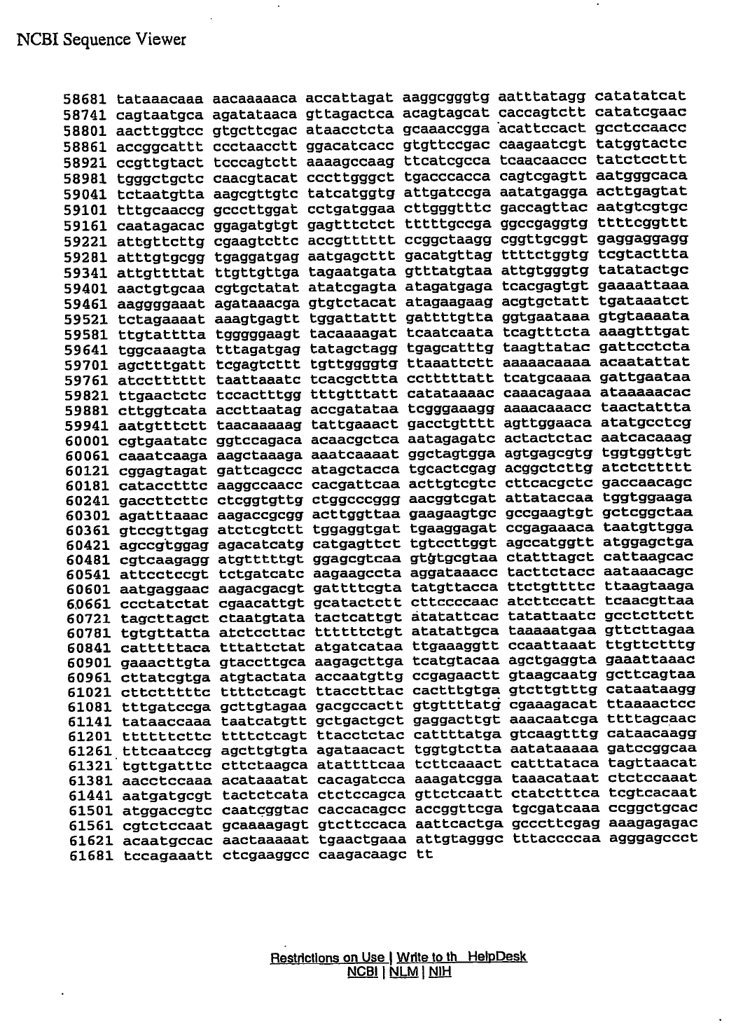

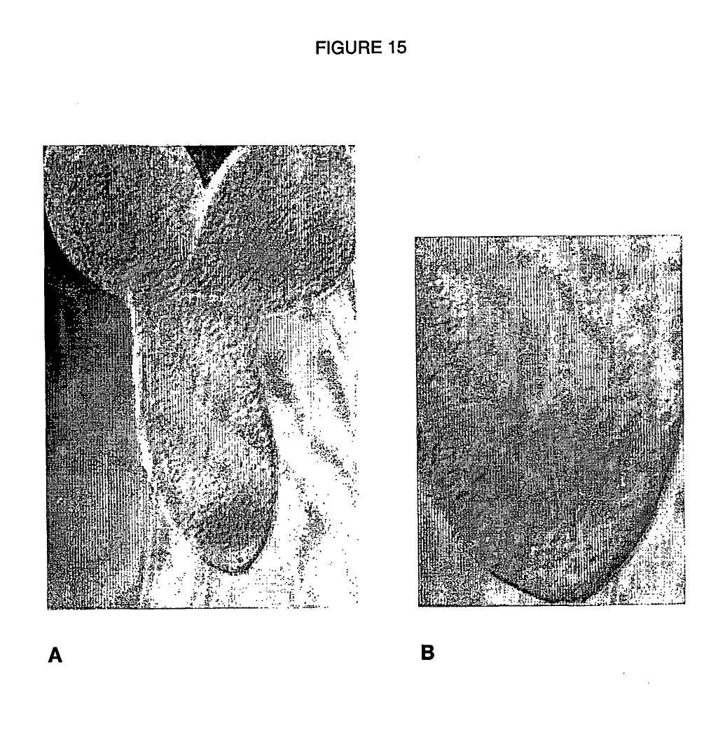

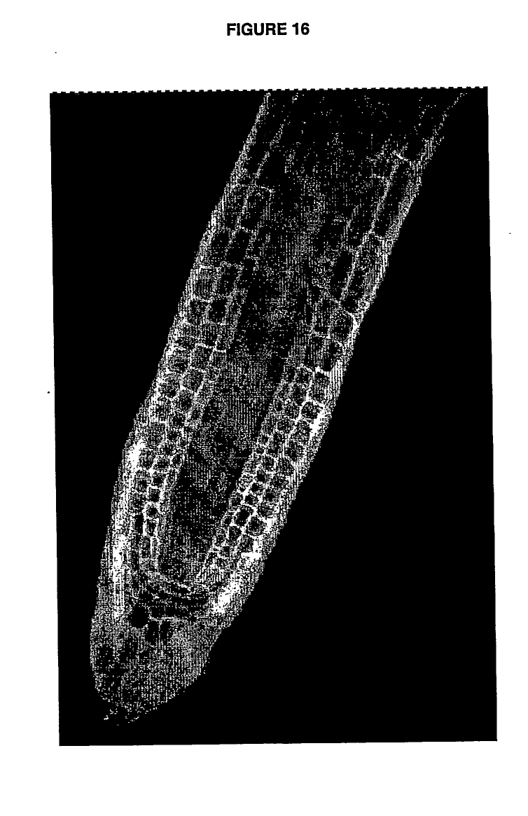

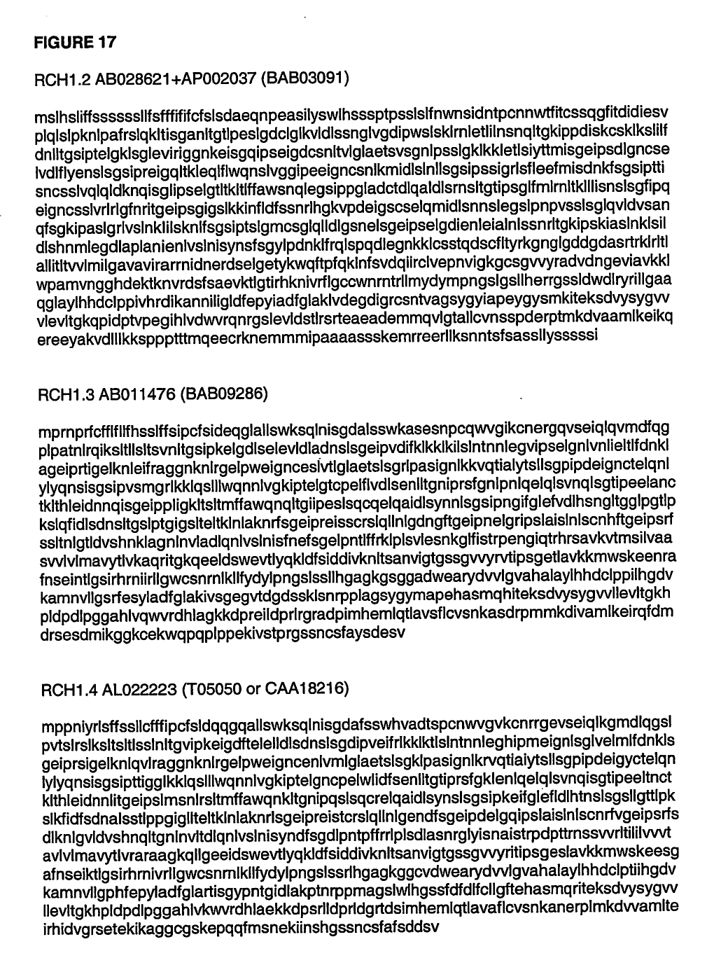

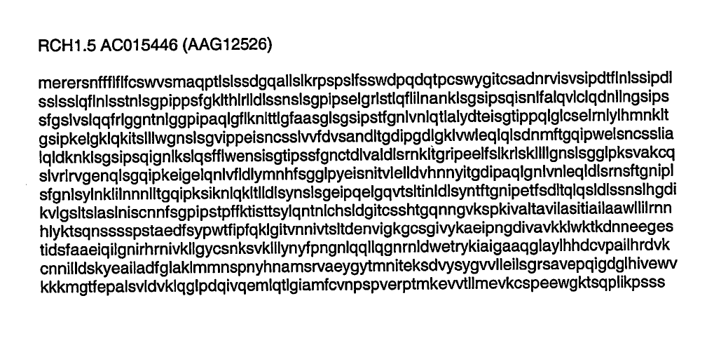

(a) In the root meristem, the quiescent center (QC) functions as an organizing center. Those cells in immediate contact with the QC are stem cells (dark shading), which are initials for all cell files: endodermis (end), cortex (cort), epidermis (epi), lateral root cap (Irc) and columella (col). (b) in the shoot meristem, WUS is functionally equivalent to the QC in the root meristem in maintaining the presence of stem cells (dark shading). Long-range signaling for stem cell maintenance is also postulated for ZLL, which is expressed in the vascular cells. CLV3 is expressed in the presumptive stem cells that the initials for the L1 (epidermis), L2 (subepidermal tissues, i.e. mesophyll cells) and L3 (subepidermal tissues, i.e. vascular cells) tissue layers (Schnittger, Schellmann, & Hulskamp 1999). CLV1 is found in the L3 tunica layer of the central zone.

FIG. 2. List of the degenerative primers used for RT-PCR of root-specific members of the LRR receptor kinase family.

FIG. 3. Differential RT-PCR on hypocotyl/cotyledon (C) and roottip (R)RNA.

The PCR fragments obtained upon amplification of R and C cDNAs with degenerate primers are separated on agarose gel. DNA fragments of different size can be detected in the R₂₂ compared to C₂₂ PCRs.

M represents the 100 bp marker (Boehringer). C and R indicated the origin of the cDNA sample used in the PCR and the numbers below indicate the primer combination used, e.g. C₂₃ represents hypocotyl/cotyledon cDNA amplified with primers LRR2 and KIN3.

FIG. 4. RT—PCR analysis of RCH1 expression.

Total RNA was isolated from flower (F), hypocotyl/cotyledon (C) and roottips (R) and RCH1 mRNA was amplified for 26 cycles with RT-PCR using the RCH1F and RCH1R primers. As a control for cDNA quality and quantity as well as relative expression level, ubiquitin (Ubi) mRNA was amplified for 20 cycles under the same conditions using the UBIF and UBIR primers.

FIG. 5. The nucleotide sequence of the RCH1 operon.

This figure shows het nucleotide sequence of SEQ ID NO 1 that contains the genomic RCH1 clone.

FIG. 6. The nucleotide sequence of the RCH1 promoter region.

This fragment of the genomic RCH1 clone is about 3498 base pairs and is identified as SEQ ID NO 2. Two putative ATG start codons of the RCH1 protein on position 3479-3481 and on position 3491-3493 could be identified and are represented in bold. The underlined sequence corresponds to SEQ ID NO 18.

It is also known that there is a gene in the reverse orientation in this promoter region.

This gene's open reading frame starts at the position 1759-1757 with the ATG (bold), contains two introns, and ends at position 350-348 with a TGA codon (bold). A homology search of the protein sequence of this antisense gene in the RCH1prom against the protein database with the program BLASTP 2.0.8 (Altschul et al. 1997) produced a hypothetical protein (Accession number AC002392, clone T20K24.8) that is a putative anthranilate N-hydroxycinnamoyl/benzoyltransferase from Arabidopsis thaliana. Both sequences show 38% identity and 54% of their amino acids are homologous.

FIG. 7. Promoter activity of the RCH1 promoter—GUS fusion construct in the Arabidopsis thaliana roots.

Seedlings of different age were grown and stained with Glucuronidase (GUS). The expression of the GUS reporter gene can be seen in the root meristem of the main and the lateral roots, as well as in the vascular tissue of the root and the lateral root primordia. (A) 1 day old seeding stained over night, (B) higher magnification of A, (C) 2 days old seedling stained for 5 hours, (D) main root from 1 day old seedling stained for 1 hour, (E) lateral root primordia from 1 week old seedling stained for 5 hours, (F, G, H) lateral roots from an 8 days old seedling stained for 5 hours.

FIG. 8. The nucleotide sequence of the RCH1 gene.

This fragment of the genomic RCH1 clone is about 3960 base pairs long and is identified as SEQ ID NO 3. Two putative ATG start codons of the RCH1 protein on position 1-3 and on position 13-15 could be identified and are represented in bold. Also the TMAA stopcodon on position 3958-3960 is represented in bold. There is a putative intron estimated to range from position 2900 (CAG\hat{ }GTA) to position 3452 (CAG\hat{ }AAT) with the program http://www.CBS.DTU.DK/services/NetGene2. The putative splice-sites are underlined.

FIG. 9. The amino acid sequence of the open reading frame encoding RCH1 protein.

Two putative start methionins of the RCH1 protein on position 1 and on position 4 could be identified. An ORF of 1135 amino acids is identified and represented in SEQ ID NO 4. The ORF of 1131 amino acids and starting at the methionine on position 4

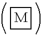

corresponds with SEQ ID NO 19.

The boxed peptide

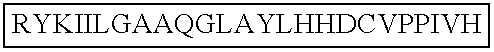

corresponds with SEQ ID NO 5.

FIG. 10. Amino acid alignment of the RCH1 protein the Arabidopsis thaliana CLV1 receptor kinase like protein.

The amino acid sequences of the RCH1 and of CLV1 (Genbank accession number AAB58929) were aligned using the program GAP as described in example 3. Match display thresholds for the alignment(s) are: |=IDENTITY,:=2,.=1.

FIG. 11. Phylogenetic tree of CLV1 homologues.

The novel RCH1 sequence was aligned with the CLV1 (Genbank accession number U96879.1), the known CLV1 homologues BRI1 (Genbank accession number AF017056), HAE (GenPept accession number S27756) and ER (Genbank accession number D83257) as well as with 4 other related proteins that were retrieved from the public database searched with the RCH1 sequence using the BlastP program. The multiple alignment and phylogenetic tree of said sequences were calculated with the Clustal V method as described in example 4. Based on the sequence, RCH1 is more homologue to CLV1 than other known CLV-homologues.

FIG. 12. Phylogenetic tree of LRR receptor like kinases

The novel RCH1 sequence was aligned with CLV1, its homologues BRI, HAE, ER and other LRR receptor like kinases retrieved from the public database. Also plant disease resistance genes, which comprise a leucine-rich repeat and which are receptor-like were included in the alignment (RPS5, RPS2, RPP8, RPM1, RPS4, N, RPP5, Meyers et al, 1999, Plant J;, November20(3): 317-32). The multiple alignment and phylogenetic tree of said proteins were calculated with the Clustal V method as described in example 4. Because of the close proximity of RCH1 to CLV1 in the phylogenetic tree, there is more evolutionary relationship between RCH1 and clavata, than with other kinds of LRR receptor like proteins such as plant disease resistance genes.

FIG. 13. The nucleotide sequence of a minimal promoter sequence of RCH1

This figure shows the nucleotide sequence of SEQ ID NO. 18 that contains a minimal promoter sequence of the RCH1 clone. This fragment of the genomic RCH1 clone is about 842 base pairs. This sequence is deducted from the large promoter region Identified as SEQ ID NO 2 (see example 7). The minimal promoter region correlates with the base pairs ranging from −1 of the first ATG defined in SEQ ID NO. 2 to 842 of the first ATG defined in SEQ ID NO. 2.

FIG. 14. Outprint of GenBank accession number AB017061 on Dec. 4, 2000.

FIG. 15. RCH1prom::GUS expression in embryo's

Arabidopsis embryo transformed with RCH1prom::GUS. Embryo's were stained with beta glucuronidase. The RCH1 promoter is active very early in plant development and is only active in the root in the embryonic phase (A). On a stronger magnification (B) it is visible that the root specific expression is limited to the meristematic zone of the embryonic root.

FIG. 16. RCH1::GFP expression in roots

RCH1 promoter is strongly active in endodermis, cortex, epidermis, lateral root cap, in short: in the “division zone”. The RCH1 promoter is also active but in a low manner, in the quiescent center and in the vascular tissue.

FIG. 17. Amino acid sequence of RCH1 homologues of Table 4

### EXAMPLES

Unless stated otherwise in the Examples, all recombinant DNA techniques are performed according to protocols as described in Sambrook et al. (1989), Molecular Cloning: A Laboratory Manual. Cold Spring Harbor Laboratory Press, NY or in Volumes 1 and 2 of Ausubel et al., (1994), Current Protocols in Molecular Biology, Current Protocols. Standard materials and methods for plant molecular work are described in Plant Molecular Biology Labfase (1993) by R. D. D. Croy, jointly published by BIOS Scientific Publications Ltd. (UK) and Blackwell Scientific Publications (UK).

### Example 1

### Isolation of the RCH1 Operon From Root Tissue of Arabidopsis thaliana

Plant organ development depends on the presence of distally positioned groups of continuously dividing cells, the shoot and root apical meristems. Laser ablation studies suggest that a balance between signals for proper differentiation and short range signals from the mitotically inactive quiescent centre (QC), required to keep cells less differentiated, is important to maintain the root meristem (van den Berg et al. 1995; van den Berg et al. 1997). In the shoot meristem a balance between the WUSCHEL and CLAVATA (CLV) genes has been implicated to play an essential role in regulation cell division and differentiation (Clark et al. 1993; Fletcher et al. 1999; Schoof et al. 2000). The CLV1 gene encodes a leucine rich repeat (LRR) receptor kinase (Clark, Williams, & Meyerowitz 1997), a member of a large gene family (Lease, Ingham, & Walker 1998). Other members are ERECTA (ER) (Torii et al. 1996), BRASSINOLIDE INSENSITVE (BRI) (Li et al. 1997) and HAESA (HAE) (Jinn et al. 2000), genes which have a function in plant development

We investigated whether root specific CLV1 homologs exist using a differential RT-PCR based approach. PCR products were generated from root tip and hypocotyl/cotyledon cDNA pools using degenerate primers, resulting in the isolation of the root specific ROOT CLAVATA HOMOLOG 1 (RCH1). The promoter region of this gene was translationally fused to β-glucoronidase-green fluorescent protein (GUS/GFP) conferring root specific expression of these reporter genes (see Example 2).

Designing Degenerate Primers

To RT-PCR root specific members of the LRR receptor kinase gene family we compared the sequences of the CLV1, ER and HAE genes and their encoded proteins. The degenerate LRR primers were designed against the NxLxGxIP encoding region of the xLxxNxLxGxIPxxLxxLxxLxxL consensus for the LRR receptor-like kinases. Degenerate kinase primers were disigned for different homologous regions of the kinase domain. The primers are listed in FIG. 2.

RNA Isolation

For each RNA isolation the roottips and the hypocotyl/cotyledon part including the shoot apical meristem of Arabidopsis thaliana (CoI0) plants, 4 days after germination, were collected.

Total RNA was isolated (Pawlowski et al. 1994) and chromosomal DNA contamination was removed upon Dnase I (Promega, Madison, Wis.) treatment, following the instructions of the suppliers. The amount and quality of RNA was determined using spectrophotometer and agarose gel electrophoresis (data not shown).

Differential RT-PCR

Reverse transcription (RT) was performed using 5 μg of root tip (R) and hypocotyl/cotyledon (C) total RNA in a 20 μl reaction volume with 0.5 μg oligodT₁₂₋₁₈ (Pharmacia, N.J., USA) and 200 U Superscript II (LifeTechnologies, Paisley, UK), according to the instructions of the supplier. Following first strand cDNA synthesis the cDNA samples were treated with RNase H (LifeTechnologies) as instructed by the supplier and subsequently diluted to a total volume of 100 μl with water.

Amplification reactions were performed with all degenerate LRR and KIN primer pairs (FIG. 2). For the polymerase chain reaction (PCR) 2.5 μl of the R or C cDNA sample was amplified in a total volume of 50 μl reaction volume containing 100 μM dNTPs, 100 ng LRR and KIN primer, 1 U Taq polymerase (Boehringer, Ingelheim, Germany) and its accompanying buffer. The cDNAs were amplified during 40 cycles (1 min at 94° C., 1 min at 45-55° C., 3 min at 72° C.).

Cloning and Sequencing

The amplified R and C cDNAs were separated and compared with agarose gel electrophoresis (FIG. 3). The root specific fragments, e.g. fragments present in R₂₂ and not in C₂₂, were isolated from gel, cloned into the pGEM-T vector (Promega) and transformed into E. coli (strain DH5α), using standard cloning procedures.

Several PCR fragments of the same size but from different members of the LRR receptor kinase gene family may be generated using degenerate primer pairs. Therefore, 24 randomly picked colonies obtained after transformation were used in a “colony PCR”. Aliquots of the amplified inserts were separated on agarose gel followed by Southern transfer to a Supercharge N+ membrane (Schleicher & Schuell, Dassel, Germany) to create a “colony blot”. An aliquot of one insert was labeled with ³²P-dCTP and hybridized to the colony blot to determine the number of clones it represented in the pool of 24. This procedure was repeated with other labeled inserts until all clones were identified.

A single representative of each pool of clones was sequenced and the sequence was analyzed using the BLAST program. One of the clones sequenced was named RCH1 and showed strong homology to plant LRR receptor kinases.

RT-PCR

The relative expression levels of RCH1 in root tip (R), hypocotyl/cotyledon (C) and flowers (F) were determined using RT-PCR. Primers specific for the RCH1 sequence were designed (RCH1 F and RCH1 R, FIG. 2). The PCR samples were analyzed with agarose gel electrophoresis. Ubiquitin (Ubi) mRNA was used as an internal control (Horvath et al. 1993). Polyubiquitin genes consist of multiple units (Callis et al. 1995) and the Ubi primers hybridize with the ends of each single unit.

Independently isolated total RNA was reverse transcribed to obtain cDNA and for the PCR reaction 2 μl of the cDNA sample was amplified in a total volume of 50 μl as described above for the differential RT-PCR. The RCH1 and Ubi cDNAs were amplified during 26 or 20 cycles (30 sec at 94° C., 30 sec at 56° C., 30 sec at 72° C.), respectively (FIG. 4). RCH1 expression was only observed in root tip (R).

### Example 2

### Promoter Isolation and Promoter Fusion Construct

To determine if the RCH1 promoter region is sufficient to confer root specific expression of a “gene of interest” we decided to fuse this promoter to the Glucuronidase/Green Fluorescent Protein (GUS/GFP) reporter gene. The RCH1 clone was used to isolate a phage containing the corresponding genomic region from a λGEM (Promega, Madison, Wis.) based genomic library. The λ-GEM11 phage containing an insert of about 14550 nucleotides comprising the Arabidopsis thaliana RCH1 promoter and RCH1 gene and also the surrounding genomic sequences was, deposited on Oct. 25, 2000 at the BCCM-LMBP plasmid collection and was named “lambda phage RCH1 genomic”. This deposit was given the accession number LMBP 5582CB by the international depositary authority. A restriction map was constructed of the genomic clone and a 7 kb XhoI fragment, containing 3.5 kb promoter and part of the RCH1 coding sequence, was subcloned into the expression vector pBS (Stratagene, La Jolla, Calif.). The 3.5 kb promoter region including the first amino acids of the putative RCH1 ORF is set forth in FIG. 5 and is identified as SEQ ID NO 2. This promoter region was amplified using the primers M13F and PRCH1 R and Pfu Taq polymerase (Stratagene, La Jolla, Calif.) in the manufacturer specified conditions. The promoter region was subsequently cloned into the binary pCAMBIA3383Xb vector (Roberts et al. 2000) creating a translational fusion with the GUS/GFP reporter gene.

GUS histochemical Assay.

Arabidopsis thaliana plants were transformed with this construct according to the vacuum transformation method (Bechtold & Pelletier 1998). Transformants were selected for phosphinothricin-tripeptide (PUT, Duchefa, NL) resistance.

Histochemical localization of β-Glucuronidase activity (GUS) was performed using the substrate 5-bromo-4-chloro-3-indolyl glucuronide (X-gluc, Biosynth A G, Staad, Switzerland). X-gluc was dissolved in n-dimethyl-formamide and diluted to 0.5 mg/ml in 50 mM NaPO4, pH 7.2 with 0.1% Triton X-100. Oxidative dimerization of the produced indoxyl derivative was enhanced by adding the oxidation catalyst K⁺ ferricyanide/ferrocyanide to a final concentration of 0.5 mM (0.5 mM K4Fe(CN)₆.H₂O/0.5 mM K3Fe(CN)₆). The GUS reaction was incubated at 37° C. over several time periods, and pictures were taken from the cloured plant-tissues (FIGS. 7 and 15). In FIG. 7 the very specific expression pattern of the RCH1promoter is visualized by the GUS reporter gene activity. Persons skilled in the art will also agree to the fact that the expression level of the RCH 1 promoter is very high, since after one hour the saturation level of staining is reached in the meristem of the main root from a 1 week old seedling (FIG. 7D).

RCH1::GFP expression in roots is shown in FIG. 16.

### Example 3

### Alignment

Sequence alignment of one complete register sequence against a target sequence as in FIG. 10 was done using the program GAP of the GCG package. The algorithm of Needleman and Wunsch is applied here to find the alignment of two complete sequences (Needleman and Wunsch, JMB 48(3): 443-453, 1970). The used parameters during the alignment were Gap Weight: 8, Average Match: 2.912, Length Weight: 2, Average Mismatch: 2.003, Quality: 1388, Length: 1149, Ratio: 1.416, and Gaps: 25.

### Example 4

### Phylogenetic Tree

For sequence comparison of proteins as in FIGS. 11 and 12, the Clustal V method to align all the sequences was used (Higgins D G & Sharp P M, Comput Appl Biosci. 1989 April;5(2):151-3). The Clustal Method groups sequences into clusters by examining sequence distances between all pairs. Clusters are aligned as pairs then collectively as sequence groups to produce the overall alignment. After the multiple alignment is completed, a neighbor-joining method is employed to reconstruct phylogeny for the putative alignment.

The Clustal method uses weight tables to construct multiple alignments. Residue weight tables are used in scoring protein and nucleotide alignments so that slightly mismatched residues, such as lie vs. Val amino acids or G vs. R nucleotides, are not scored the same as total mismatches. For the alignment and pylogenetic trees in FIGS. 11 and 12 the PAM250 weight table was used. Other method parameters for pairwise alignment were K-tuple: 1, Gap Penalty: 3, Window: 5 and Diagonals: 5. Other method parameters for multiple alignment include Gap Penalty: 10 and Gap Length Penalty: 10.

To view phylogenetic relationships compatible with multiple sequence alignments a Phylogenetic Tree was calculated. The length of each pair of branches represents the distance between sequence pairs, while the units at the bottom of the tree indicate the number of substitution events. The phylogenetic tree gives a first estimate of the relationship between homologous sequences. A cladogram presentation of the phylogenetic tree is shown in FIGS. 11 and 12.

### Example 5

### Root-Specific Report r Gene Expression in Rice Mediated by the Arabidopsis thaliana RCH 1 Promoter

CLAVATA homologs are found in many other plant species from both the groups of monocots and dicots. Therefore one can assume that also the promoter of RCH1 is functional in those other plant species, and possibly exerts the same specific expression-pattern. To investigate the feasibility of root meristem-specific activation of the Arabidopsis thaliana RCH1 promoter in monocots, the aforementioned promoter-reporter gene construct, wherein the GUS/GFP chimeric gene is operably linked to the RCH1 promoter, will be transformed to rice using the standard transformation procedures well known to the persons skilled in the art and outlined in the following paragraph. After several time periods ranging from 1 day to 1 or more weeks, the seedling will be checked for the expression of the GUS reporter gene. This will be done by growing the seedlings in organogenesis medium, and staining them with glucuronidase for several time periods ranging from 1 hour to 5 hours.

Agrobacterium-Mediated Rice Transformation

The Promoter-GUS hybrid gene outlined in Example 2 can be transformed to Agrobacterium tumefaciens strain LBA4404 or C₅₈ by means of electroporation and subsequently transformed bacterial cells can be selected on a solid agar medium containing the appropriate antibiotics.

For demonstration of root-specific GUS expression with the promoter of the current invention, 309 mature dry seeds of the rice japonica cultivars Nipponbare or Taipei are dehusked, sterilised and germinated on a medium containing 2,4-dichlorophenoxyacetic acid (2,4-D). After incubation in the dark for four weeks, embryogenic, scutellum-derived calli are excised and propagated on the same medium. Selected embryogenic callus is then co-cultivated with Agrobacterium. Co-cultivated callus is grown on 2,4-D-containing medium for 4 to 5 weeks in the dark in the presence of a suitable concentration of the appropriate selective agent. During this period, rapidly growing resistant callus islands develop. After transfer of this material to a medium with a reduced concentration of 2,4-D and incubation in the light, the embryogenic potential is released and shoots develop in the next four to five weeks. Shoots are excised from the callus and incubated for one week on an auxin-containing medium from which they can be transferred to the soil. Hardened shoots are grown under high humidity and short days in a phytotron. Seeds can be harvested three to five months after transplanting. The method yields single locus transformants at a rate of over 50% (Chan et al. 1993; Hiei et al. 1994)

### Example 6

### Expression of a Cytokinine Oxidase cDNA Under the Root-Specific RCH1 Promoter in Transgenic Tobacco and Rice Leads to increased Root Production

The AtCKX1 gene (located in the database with the accession number: AC002510, Arabidosis thaliana chromosome II section 225 of 255 of the complete sequence, sequence from clone T32G6) can be cloned under the control of the RCH1 promoter of Arabidopsis, in a binary vector. Also the ZmCKX1 from maize (accession number AF044603, (Morris et al. 1999) can be operably linked to the RCH1 promoter a binary vector. These genes can be transfected into Tobacco or Rice plants, using agrobacterium-mediated gene transfer. The transgenic plants expressing the ATCKX1 or the ZmCKX1 specifically in roots are expected to show increased root production without negatively affecting shoot development and without inducing premature leaf senescence.

### Example 7

### Minimal Promoter Sequence

The length of the RCH1 promoter region (SEQ ID NO 2), which confers to root specific expression patterns, can be reduced to a “minimal promoter” without altering the root specific expression patterns or other features of the promoter such as constitutive and strong activity. As an example, the length of the promoter region was reduced to a minimal promoter sequence ranging from the −1 base pair just before the start codon at position 3479-3481 of SEQ ID NO. 2 to −842 base pairs before said start codon. Said minimal promoter is represented in SEQ ID NO. 18. This minimal promoter was PCR amplified and translationally fused to the GUS reporter gene. With a GUS histochemical assay (example 2) the same expression pattern as the large promoter region was demonstrated (results not shown). The difference is that the sequence before the HindIII site at the beginning of the minimal promoter is not necessary for the root specific expression pattern.

### Example 8

### RCH1 prom::GUS Expression in Embryo's

Siliques of Arabidopsis plants transformed with RCH1prom::GUS were opened at different stages of development and the embryo's were surgically taken out of the ovules and transferred to X-gluc staining solution. Embryo's were stained for 0.5h-4h before pictures were taken using light microscopy, FIGS. 15A and B are at 25× and 40× magnification respectively, stained for 0.5 h. Longer staining did not reveal expression outside the root promeristem region.

RCH1 is active very early: RCH1 expression was first observed in torpedo stage embryo's in the root promeristem and was maintained in this region during the later stages of development. This figure shows that as soon as a meristem is formed RCH1 is expressed and provides further evidence that RCH1 expression is correlated with the appearance and presence of a root meristem from the embryonic stage onwards.

### Example 9

### Isolation of cDNA of Other Arabidopsis RCH1 Homologues

TABLE 4
% identity with RCH1

| | | | % sequence |
|---|---|---|---|
| | Database entry | Database entry | identity with |
| | genomic DNA | protein | RCH1 on aa level |
| RCH1.2 | AB028621 + AP002037 | BAB03091 | 59.8% |
| RCH1.3 | AB011476 | BAB09286 | 40.1% |
| RCH1.4 | AL022223 | CAA1826 or | 38.9% |
| | | T05050 | |
| RCH1.5 | AC015446 | AAG12526 | 42.1% |

To discover the exact function of RCH1, the inventors want to circumvent the redundancy of the gene.

AB028621 (see FIG. 12, phylogenetic tree) is determined to be RCH1.2 protein and is also used by the inventors for further experiments. Double knock outs of RCH1 and RCH1.2 were made and also other closely related sequences (AB011476.pro, T05050.pro and AC015446.pro) are used in knock out experiments or in antisens expression.

Ectopic Expression of RCH1

The expression of RCH1 in normal cells correlates with the zone of rapidly dividing cells s and is involved in the maintenance of meristem cells and dividing cells. Improved activity of RCH1 in a host cell results in the maintenance of cell division in that host cell and thus works against the differentiation of that cell. On the contrary diminished RCH1 activity in a host cell results in a diminished cell division in that host cell and thus in the loss of the amount of dividing cells in the meristem. Particular examples to influence these processes by altering the expression level of the RCH1 gene are described in the following paragraphs:

Overexpression

In order to obtain higher activity of the RCH1 protein in the host cell the coding region of the RCH1 gene is fused to a strong promoters that is inducible or constitutive and/or ubiquitous or tissue specific.

To influence the RCH1 activity in a particular set of host cells, the RCH1 coding region is cloned under control of other meristemic promoters such as the promoters of WUSCHEL, CLV1, CLV2, CLV3, STM. The obtained effect is the control of the expression of RCH1 in a particular area of the meristem or in a particular time period of the development of the plant. Also the promoters of other RCH genes (such as AB028621.pro, AB011476.pro, T05050.pro and AC015446.pro) are used for this purpose.

In a particular application of the present invention, the RCH1 gene and the RKN gene (=the rice homologue of RCH1 as described in WO/04761) are operationally linked to the root-specific promoter PYK10, a constitutive ubiquitin or GOS 2 promoter and Cdc2a promoter and expressed in Arabidopsis or in rice.

Downregulation

In order to obtain lower activity of the RCH1 protein in the host cell, the coding region of the RCH1 gene cloned in a sense and antisense orientation in order to form a hairpin that induces gene-silencing in the host cell.

Co-Expression

Downregulation or upregulation of the RCH1 in combination with downregulation or upregulation of other genes e.g.:

With WUSHEL:

In the shoot meristem a balance between the WUSCHEL and CLAVATA (CLV) genes has been implicated to play an essential role in regulation cell division and differentiation (Clark et al. 1993; Fletcher et al. 1999; Schoof et al. 2000). The whole meristem maintenance is controlled by these genes and they seem to have an opposite function keeping the balance between cell division and differentiation. Therefore, WUSCHEL or a root WUSCHEL homologue is a preferred partner to be co-expressed with RCH1.

In an application of the invention RCH1 is upregulated in combination with the downregulation of WUSCHEL in a root specific manner. The obtained effect of this co-expression is a better control on the meristem maintenance.

With Other RCH1 Family Members

Possibly the function of RCH1 is redundant and other closely related genes can take over its function. Therefore the RCH1 gene ectopically expressed in combination with closely related genes in order to downregulate or upregulate the activity of the encoded protein. The RCH1 homologues used for co-expression are for example AB028621.pro, AB011476.pro, T05050.pro and AC015446 (see FIG. 12 and table 4). The obtained effect is a better effect as described for the ectopic expression of RCH1 alone.

With Other Clavata Homologues:

Clavata 1 is a receptor that is involved in a signal pathway probably through the binding of a ligand (e.g. Clavata 3). A particular useful application of the present invention is the co-expression of RCH1 with the root Clavata 3 homologue RCH3. The obtained effect is a better control on meristem maintenance.

### Example 10

### Domain Swapping

The N-terminal LRR domain of RCH1 functions as an extracellular perception domain and a C-terminal kinase domain functions in an intracellular signal transduction pathway. Previously it was described by He et al. (Science 2000, 288(5475):2360-3: Perception of brassinosteroids by the extracellular domain of the receptor kinase BRI1) that such domains can be swapped with the domains of other proteins. Following this methodology in a particular application of the present invention, the domains of RCH1 are swapped with the domains of another protein. In one example the LRR domain of RCH1 is replaced by the LRR domain of BRI. The effect obtained is the activation of a different pathway, which is normally not activated by RCH1, because the kinase domain of RCH1 now responds to another external signal that normally is not recognized by RCH1.

It will be clear that the invention may be practiced otherwise than as particularly described in the forgoing description and examples. Numerous modifications and variations of the present invention are possible in light of the above teaching and, therefore, are within the scope of the appended claims.

### REFERENCES

Alberts, B. et al. (1994) Molecular Biology of the Cell, Garland Publishing Inc.

Altschul S. F. et al. (1997) Gapped BLAST and PSI-BLAST: a new generation of protein database search programs. Nucleic Acids Res. 25, 3389-3402.

An G. et al. (1985) New cloning vehicles for transformation of higher plants. EMBO J. 4, 277-284.

Armstrong C. L. et al. (1990) Factors affecting PEG-mediated stable transformation of maize protoplasts. Plant Cell Reports 9, 335-339.

Banerjee A. et al. (1996) Omega amino acids in peptide design: incorporation into helices. Biopolymers 39, 769-777.

Baron M. H. & Baltimore D. (1982) Antibodies against the chemically synthesized genome-linked protein of poliovirus react with native virus-specific proteins. Cell 28, 395-404.

Bartel, P. L. & Fields, S. (1997) The Yeast Two-Hybrid System, Oxford University Press.

Baszczynski, C. L., Fallis, L., Bellemare, G., and Boivin, R. Brassica regulatory sequence for root-specific or root-abundant gene expression. WO9007001. 2000.

Bechtold N. & Pelletier G. (1998) In planta Agrobacterium-mediated transformation of adult Arabidopsis thaliana plants by vacuum infiltration. Methods Mol. Biol. 82, 259-266.

Bell M. H. et al. (1993) Tobacco plants transformed with cdc25, a mitotic inducer gene from fission yeast. Plant Mol. Biol 23, 445-451.

Benkirane N. et al. (1996) Exploration of requirements for peptidomimetic immune recognition. Antigenic and immunogenic properties of reduced peptide bond pseudopeptide analogues of a histone hexapeptide. J. Biol. Chem. 271, 33218-33224.

Berry A. & Brenner S. E. (1994) A prototype computer system for de novo protein design. Biochem. Soc. Trans. 22, 1033-1036.

Binarova P. et al. (1998) Treatment of Vicia faba root tip cells with specific inhibitors to cyclin-dependent kinases leads to abnormal spindle formation. Plant J. 16, 697-707.

Bögre L. et al. (1997) The cdc2Ms kinase is differentially regulated in the cytoplasm and in the nucleus. Plant Physiol. 113, 841-852.

Bögre L. et al. (1999) A MAP kinase is activated late in plant mitosis and becomes localized to the plane of cell division. Plant Cell 11, 101-113.

Borg S. et al. (1997) Identification of new protein species among 33 different small GTP-binding proteins ncoded by cDNAs from Lotus japonicus, and expression of corresponding mRNAs in developing root nodules. Plant J. 11, 237-250.

Calderini O. et al. (1998) A cell cycle regulated MAP kinase with a possible role in cytokinesis in tobacco cells. J. Cell Sci. 111 (Pt 20), 3091-3100.

Callis J. et al. (1995) Structure and evolution of genes encoding polyubiquitin and ubiquitin like proteins in Arabidopsis thaliana ecotype Columbia. Genetics 139, 921 939.

Chan M. T. et al. (1993) Agrobacterium-mediated production of transgenic rice plants expressing a chimeric alpha-amylase promoter/beta-glucuronidase gene. Plant Mol. Biol. 22, 491-506.

Chaubet N. et al. (1996) Identification of cis-elements regulating the expression of an Arabidopsis histone H4 gene. Plant J. 10, 425-435.

Christou P., McCabe D. E., & Swain W. F. (1988) Stable transformation of soybean callus by DNA-coated gold particles. Plant Physiol. 87, 671-674.

Clark S. E., Running M. P., & Meyerowitz E. M. (1993) CLAVATA1, a regulator of meristem and flower development in Arabidopsis. Development 119, 397-418.

Clark S. E., Williams R. W., & Meyerowitz E. M. (1997) The CLAVATA1 gene encodes a putative receptor kinase that controls shoot and floral meristem size in Arabidopsis. Cell 89, 575-585.

Cohen-Fix O. & Koshland D. (1997) The metaphase-to-anaphase transition: avoiding a mid-life crisis. Curr. Opin. Cell Biol 9, 800-806.

Colasanti J., Tyers M., & Sundaresan V. (1991) Isolation and characterization of cDNA clones encoding a functional p34cdc2 homologue from Zea mays. Proc. Natl. Acad. Sci. U.S.A 88, 3377-3381.

Crossway A. et al. (1986) Integration of foreign DNA following microinjection of tobacco mesophyll protoplasts. Mol. Gen. Genet 202,179-185.

Dale E. C. & Ow D. W. (1990) Intra- and intermolecular site-specific recombination in plant cells mediated by bacteriophage P1 recombinase. Gene 91, 79-85.

de Pater B. S. & Schilperoort R. A. (1992) Structure and expression of a root-specific rice gene. Plant Mol. Biol. 18, 161-164.

De Veyider L. et al. (1997) The Arabidopsis Cks1At protein binds the cyclin-dependent kinases Cdc2aAt and Cdc2bAt. FEBS Lett. 412, 446-452.

Dodds, J. H. (1985) Plant genetic engineering, Cambridge University Press.

Doemer P. et al. (1996) Control of root growth and development by cyclin expression. Nature 380, 520-523.

Dolferus R. et al. (1994) Differential interactions of promoter elements in stress responses of the Arabidopsis Adh gene. Plant Physiol 105, 1075-1087.

Dorner B. et al. (1996) The synthesis of peptidomimetic combinatorial libraries through successive amide alkylations. Bioorg. Med. Chem. 4, 709-715.

Elledge S. J. (1996) Cell cycle checkpoints: preventing an identity crisis. Science 274, 1664-1672.

Ellis J. G. et al. (1987) Maize Adh-1 promoter sequences control anaerobic regulation: addition of upstream promoter elements from constitutive genes is necessary for expression in tobacco. EMBO J. 6, 11-16.

Ely, S., Evans, I. J., and Schuch, W. W. Root-specific promotor. 288630. 2000. 8-10-1994. Ref Type: Patent

Evans T. et al. (1983) Cyclin: a protein specified by maternal mRNA in sea urchin eggs that is destroyed at each cleavage division. Cell 33, 389-396.

Fantes P. (1989) Yeast cell cycle. Curr. Opin. Cell Biol 1, 250-255.

Fassina G. & Melli M. (1994) Identification of interactive sites of proteins and protein receptors by computer-assisted searches for complementary peptide sequences. Immuno methods. 5, 114-120.

Fedoroff N. V. & Smith D. L. (1993) A versatile system for detecting transposition in Arabidopsis. Plant J. 3, 273-289.

Feiler H. S. & Jacobs T. W. (1990) Cell division in higher plants: a cdc2 gene, its 34-kDa product, and histone H1 kinase activity in pea. Proc. Natl. Acad. Sci. U.S.A 87, 5397-5401.

Fesquet D. et al. (1993) The MO15 gene encodes the catalytic subunit of a protein kinase that activates cdc2 and other cyclin-dependent kinases (CDKs) through phosphorylation of Thr161 and its homologues. EMBO J. 12, 3111-3121.

Fletcher J. C. et al. (1999) Signaling of cell fate decisions by CLAVATA3 in Arabidopsis shoot meristems. Science 283, 1911-1914.

Fowler T. J., Bernhardt C., & Tierney M. L. (1999) Characterization and expression of four proline-rich cell wall protein genes in Arabidopsis encoding two distinct subsets of multiple domain proteins. Plant Physiol 121, 1081-1092.

Francis D. & Halford N. G. (1995) The plant cell cycle. Physiol. Plant. 93, 365-374.

Francis, D., Dudits, D., & Inzé, D. (1998) Plant Cell Division, Portland Press London.

Fromm M., Taylor L. P., & Walbot V. (1985) Expression of genes transferred into monocot and dicot plant cells by electroporation. Proc. Natl. Acad. Sci. U.S.A 82, 5824-5828.

Giritch A. et al. (1997) A root-specific iron-regulated gene of tomato encodes a lysyl-tRNA-synthetase-like protein. Eur. J. Biochem. 244, 310-317.

Goddemeier M. L., Wulff D., & Feix G. (1998) Root-specific expression of a Zea mays gene encoding a novel glycine rich protein, zmGRP3. Plant Mol. Biol. 36, 799-802.

Gorthy P. & Pudukottah M. G. (1999) Production of silicon carbide from rice husks. J. Am. Ceramic Soc. 82.

Gubler F. & Jacobsen J. V. (1992) Gibberellin-responsive elements in the promoter of a barley high-pl alpha-amylase gene. Plant Cell 4, 1435-1441.

Hansen G. & Chilton M. D. (1996) “Agrolistic” transformation of plant cells: integration of T-strands generated in planta. Proc. Natl. Acad. Sci. U.S.A 93, 14978-14983.

Hansen G., Shillito R. D., & Chilton M. D. (1997) T-strand integration in maize protoplasts after codelivery of a T-DNA substrate and virulence genes. Proc. Natl. Acad. Sci. U.S.A 94,11726-11730.

Hansen G., Shillito R. D., & Chilton M. D. (1997) T-strand integration in maize protoplasts after codelivery of a T-DNA substrate and virulence genes. Proc. Natl. Acad. Sci. U.S.A 94,11726-11730.

Hanson B. et al. (1999) A simple method to enrich an Agrobacterium-transformed population for plants containing only T-DNA sequences. Plant J. 19, 727-734.

Harlow, E. & Lane, D. (1988) Antibodies: A Laboratory Manual, Cold Spring Harbor Laboratory Press.

Haseloff J. et al. (1997) Removal of a cryptic intron and subcellular localization of green fluorescent protein are required to mark transgenic Arabidopsis plants brightly. Proc. Natl. Acad. Sci. U.S.A 94, 2122-2127.

Hayles J. & Nurse P. (1986) Cell cycle regulation in yeast. J. Cell Sci. Suppl 4, 155-170.

Helariutta Y. et al. (2000) The SHORT-ROOT gene controls radial patterning of the Arabidopsis root through radial signaling [In Process Citation]. Cell 101, 555-567.

Hemerly A. S. et al. (1993) cdc2a expression in Arabidopsis is linked with competence for cell division. Plant Cell 5, 1711-1723.

Herrera-Estrella L. et al. (1983) Chimeric genes as dominant selectable markers in plant cells. EMBO J. 2, 987-995.

Herrera-Estrella L. et al. (1983) Expression of chimaeric genes transferred into plant cells using a Ti-plasmid-derived vector. Nature 303, 209-213.

Hiei Y. et al. (1994) Efficient transformation of rice (Oryza sativa L.) mediated by Agrobacterium and sequence analysis of the boundaries of the T-DNA. Plant J. 6, 271-282.

Hirt H. et al. (1991) Complementation of a yeast cell cycle mutant by an alfalfa cDNA encoding a protein kinase homologous to p34cdc2. Proc. Natl. Acad. Sci. U.S.A 88, 1636-1640.

Hirt H. et al. (1993) cdc2MsB, a cognate cdc2 gene from alfalfa, complements the G1/S but not the G2/M transition of budding yeast cdc28 mutants. Plant J. 4, 61-69.

Hochstrasser M. (1998) There's the rub: a novel ubiquitin-like modification linked to cell cycle regulation. Genes Dev. 12, 901-907.

Hoffman D. L. et al. (1995) Rapid protein structure classification using one-dimensional structure profiles on the bioSCAN parallel computer. Comput. Appl. Biosci. 11, 675-679.

Horvath B. et al. (1993) Lipo-oligosaccharides of Rhizobium induce infection-related early nodulin gene expression in pea root hairs. Plant J. 4, 727-733.

Huntley R. et al. (1998) The maize retinoblastoma protein homologue ZmRb-1 is regulated during leaf development and displays conserved interactions with G1/S regulators and plant cyclin D (CycD) proteins. Plant Mol. Biol. 37, 155-169.

Hwang I. & Goodman H. M. (1995) An Arabidopsis thaliana root-specific kinase homolog is induced by dehydration, ABA, and NaCl. Plant J. 8, 37-43.

Jaglo-Ottosen K. R. et al. (1998) Arabidopsis CBF1 overexpression induces COR genes and enhances freezing tolerance. Science 280, 104-106.

Jeong S., Trotochaud A. E., & Clark S. E. (1999) The Arabidopsis CLAVATA2 gene encodes a receptor-like protein required for the stability of the CLAVATA1 receptor-like kinase. Plant Cell 11,1925-1934.

Jinn T. L., Stone J. M., & Walker J. C. (2000) HAESA, an Arabidopsis leucine-rich repeat receptor kinase, controls floral organ abscission. Genes Dev. 14, 108-117.

John P. C., Sek F. J., & Lee M. G. (1989) A homolog of the cell cycle control protein p34cdc2 participates in the division cycle of Chlrnydomonas, and a similar protein is detectable in higher plants and remote taxa. Plant Cell 1, 1185-1193.

John, P. C. L. (1981) The cell cycle, Cambridge University Press.

Joung J. K., Ramm E. I., & Pabo C. O. (2000) A bacterial two-hybrid selection system for studying protein-DNA and protein-protein interactions. Proc. Natl. Acad. Sci. U.S.A 97, 7382-7387.

Keller B. & Lamb C. J. (1989) Specific expression of a novel cell wall hydroxyproline-rich glycoprotein gene in lateral root initiation. Genes Dev. 3, 1639-1646.

Kikuchi H. et al. (1999) Molecular characterization of a gene for alanine aminotransferase from rice (Oryza sativa). Plant Mol. Biol. 39, 149-159.

Krek W. (1998) Proteolysis and the G1/S transition: the SCF connection. Curr. Opin. Genet. Dev. 8, 36-42.

Krens F. A. et al. (1982) In vitro transformation of plant protoplasts with Ti-plasmid DNA. Nature 296, 72-74.

Kumagai A. & Dunphy W. G. (1991) The cdc25 protein controls tyrosine dephosphorylation of the cdc2 protein in a cell-free system. Cell 64, 903-914.

Labbe J. C. et al. (1989) MPF from starfish oocytes at first meiotic metaphase is a heterodimer containing one molecule of cdc2 and one molecule of cyclin B. EMBO J. 8, 3053-3058.

Lagarde D. et al. (1996) Tissue-specific expression of Arabidopsis AKT1 gene is consistent with a role in K+ nutrition. Plant J. 9, 195-203.

Lake R. S. & Salzman N. P. (1972) Occurrence and properties of a chromatin-associated F1-histone phosphokinase in mitotic Chinese hamster cells. Biochemistry 11, 4817-4826.

Langan T. A. (1978) Methods for the assessment of site-specific histone phosphorylation. Methods Cell Biol 19, 127-142.

Lauter F. R. (1996) Root-specific expression of the LeRse-1 gene in tomato is induced by exposure of the shoot to light. Mol. Gen. Genet. 252, 751-754.

Lease K., Ingham E., & Walker J. C. (1998) Challenges in understanding RLK function. Curr. Opin. Plant Biol. 1, 388-392.

Lee M. G. & Nurse P. (1987) Complementation used to clone a human homologue of the fission yeast cell cycle control gene cdc2. Nature 327, 31-35.

Lerner R. A. (1982) Tapping the immunological repertoire to produce antibodies of predetermined specificity. Nature 299, 593-596.

Lerber R. A. et al. (1981) Chemically synthesized peptides predicted from the nucleotide sequence of the hepatitis B virus genome elicit antibodies reactive with the native envelope protein of Dane particles. Proc. Natl. Acad. Sci. U.S.A 78, 3403-3407.

Li J. & Chory J. (1997) A putative leucine-rich repeat receptor kinase involved in brassinosteroid signal transduction. Cell 90, 929-938.

Liddle, J. E. & Cryer, A. (1991) A Practical Guide to Monoclonal Antibodies, Wiley N.Y.

Lisztwan J. et al. (1998) Association of human CUL-1 and ubiquitin-conjugating enzyme CDC34 with the F-box protein p45(SKP2): evidence for evolutionary conservation in the subunit composition of the CDC34-SCF pathway. EMBO J. 17, 368-383.

Llewellyn D. J. et al. (1987) Structure and expression of an alcohol dehydrogenase 1 gene from Pisum sativum (cv. “Greenfeast”). J. Mol. Biol. 195, 115-123.

Loffer F. D. (1994) The need to monitor intrauterine pressure—myth or necessity? [editorial; comment]. J. Am. Assoc. Gynecol. Laparosc. 2,1-2.

Long J. A. & Barton M. K. (1998) The development of apical embryonic pattern in Arabidopsis. Development 125, 3027-3035.

Lundgren K. et al. (1991) mik1 and wee1 cooperate in the inhibitory tyrosine phosphorylation of cdc2. Cell 64, 1111-1122.

Magyar A. & Mihalik R. (1997) Characterisation of the swine swC1 antigen. Acta Vet. Hung. 45, 17-31.

Malamy J. E. & Benfey P. N. (1997) Analysis of SCARECROW expression using a rapid system for assessing transgene expression in Arabidopsis roots. Plant J. 12, 957-963.

Masuda S., Sakuta C., & Satoh S. (1999) cDNA cloning of a novel lectin-like xylem sap protein and its root specific expression in cucumber. Plant Cell Physiol 40, 1177-1181.

Merrifield R. B. (1963) Solid phase peptide synthesis. I. The synthesis of a tetrapeptide. J. Amer. Chem. Soc. 85, 2149-2154.

Mironov V. et al. (1999) Cyclin-dependent kinases and cell division in plants—the nexus. Plant Cell 11, 509-522.

Monge A. et al. (1995) Computer modeling of protein folding: conformational and energetic analysis of reduced and detailed protein models. J. Mol. Biol 247, 995-1012.

Morris R. O. et al. (1999) Isolation of a gene encoding a glycosylated cytokinin oxidase from maize. Biochem. Biophys. Res. Commun. 255, 328-333.

Murakami T., Simonds W. F., & Spiegel A. M. (1992) Site-specific antibodies directed against G protein beta and gamma subunits: effects on alpha and beta gamma subunit interaction. Biochemistry 31, 2905-2911.

Murray A. W. & Kirschner M. W. (1989) Dominoes and clocks: the union of two views of the cell cycle. Science 246, 614-621.

Nasmyth K. (1993) Control of the yeast cell cycle by the Cdc28 protein kinase. Curr. Opin. Cell Biol 5, 166-179.

Norbury C. & Nurse P. (1992) Animal cell cycles and their control. Annu. Rev. Biochem. 61, 441-470.

Nurse P. & Bissett Y. (1981) Gene required in G1 for commitment to cell cycle and in G2 for control of mitosis in fission yeast. Nature 292, 558-560.

Nurse P. (1990) Universal control mechanism regulating onset of M-phase. Nature 344, 503-508.

Okada K. et al. (2000) Five geranylgeranyl diphosphate synthases expressed in different organs are localized into three subcellular compartments in arabidopsis [In Process Citation]. Plant Physiol 122, 1045-1056.

Okumura N. et al. (1994) A dioxygenase gene (Ids2) expressed under iron deficiency conditions in the roots of Hordeum vulgare. Plant Mol. Biol. 25, 705-719.

Olson D. C., Oetiker J. H., & Yang S. F. (1995) Analysis of LE-ACS3, a 1-aminocyclopropane-1-carboxylic acid synthase gene expressed during flooding in the roots of tomato plants. J. Biol. Chem. 270,14056-14061.

Olszewski K. A., Kolinski A., & Skolnick J. (1996) Folding simulations and computer redesign of protein A three-helix bundle motifs. Proteins 25, 286-299.

Onouchi H. et al. (1991) Operation of an efficient site-specific recombination system of Zygosaccharomyces rouxii in tobacco cells. Nucleic Acids Res. 19, 6373-6378.

Onouchi H. et al. (1995) Visualization of site-specific recombination catalyzed by a recombinase from Zygosaccharomyces rouxii in Arabidopsis thaliana. Mol. Gen. Genet. 247, 653-660.

Ormrod, J. C. & Francis, D. (1993) Molecular and Cell Biology of the Plant Cell Cycle, Kluwer Academic Publishers.

Osborne B. I., Wirtz U., & Baker B. (1995) A system for insertional mutagenesis and chromosomal rearrangement using the Ds transposon and Cre-lox. Plant J. 7, 687-701.

Ostresh J. M. et al. (1996) Generation and use of nonsupport-bound peptide and peptidomimetic combinatorial libraries. Methods Enzymol. 267, 220-234.

Pabo C. O. & Suchanek E. G. (1986) Computer-aided model-building strategies for protein design. Biochemistry 25, 5987-5991.

Pawlowski K. et al. (1994) Isolation of total, poly(A) and polysomal RNA from plant tissue. In: Plant Molecular Biology Manual (eds S. B. Gelvin & R. A. Schilperoort), pp. 1-13. Kluwer academic Publisher, dordrecht.

Peralta E. G., Hellmiss R., & Ream W. (1986) Overdrive, a T-DNA transmission enhancer on the A. tumefaciens tumour-inducing plasmid. EMBO J. 5, 1137-1142.

Pines J. (1995) Cell cycle. Confirmational change [news; comment]. Nature 376, 294-295.

Poon R. Y. et al. (1993) The cdc2-related protein p40MO15 is the catalytic subunit of a protein kinase that can activate p33cdk2 and p34cdc2. EMBO J. 12, 3123-3132:

Pysh L. D. et al. (1999) The GRAS gene family in Arabidopsis: sequence characterization and basic expression analysis of the SCARECROW-LIKE genes. Plant J. 18, 111-119.

Reed S. I., Hadwiger J. A., & Lorincz A. T. (1985) Protein kinase activity associated with the product of the yeast cell division cycle gene CDC28. Proc. Natl. Acad. Sci. U.S.A 82, 4055-4059.

Renaudin J. P. et al. (1996) Plant cyclins: a unified nomenclature for plant A-, B- and D-type cyclins based on sequence organization. Plant Mol. Biol. 32, 1003-1018.

Renouf D. V. & Hounsell E. F. (1995) Molecular modelling of glycoproteins by homology with nonglycosylated protein domains, computer simulated glycosylation and molecular dynamics. Adv. Exp. Med. Biol 376, 37-45.

Roberts, C. S., Rajagopal, S., Smith, L. A., Nguyen, T. A., Yang, W., Nugroho, S., Ravi, K. S., Cao, M-L., Vijayachandra, K., Patell, V., Harcourt, R. L., Dransfield, L., Desamero, N., Slamet, I., Keese, P., Kilian, A., and Jefferson, R. A. A comprehensive set of modular vectors for advanced manipulations and efficient transformation of plants by both Agrobacterium and direct DNA uptake methods. 2000. Rose R. B. et al. (1996) Three-dimensional structures of HIV-1 and SIV protease product complexes. Biochemistry 35, 12933-12944.

Russell P. & Nurse P. (1986) cdc25+ functions as an inducer in the mitotic control of fission yeast. Cell 45,145-153.

Russell P. & Nurse P. (1987) Negative regulation of mitosis by wee1+, a gene encoding a protein kinase homolog. Cell 49, 559-567.

Russell P. & Nurse P. (1987) The mitotic inducer nim1+functions in a regulatory network of protein kinase homologs controlling the initiation of mitosis. Cell 49, 569-576.

Rutenber E. E. et al. (1996) A new class of HIV-1 protease inhibitor: the crystallographic structure, inhibition and chemical synthesis of an aminimide peptide isoster Bioorg. Med. Chem. 4, 1545-1558.

Sambrook, J., Fritsch, E. F., & Maniatis, T. (1989) Molecular Cloning: A Laboratory Manual, Cold Spring Harbor Laboratory Press.

Schlappi M., Smith D., & Fedoroff N. (1993) TnpA trans-activates methylated maize Suppressor-mutator transposable elements in transgenic tobacco. Genetics 133, 1009-1021.

Schneider K. et al. (1998) The ROOT HAIRLESS 1 gene encodes a nuclear protein required for root hair initiation in Arabidopsis. Genes Dev. 12, 2013-2021.

Schnittger A., Schellmann S., & Hulskamp M. (1999) Plant cells—young at heart? Curr. Opin. Plant Biol. 2, 508-512.

Schoof H. et al. (2000) The stem cell population of Arabidopsis shoot meristems in maintained by a regulatory loop between the CLAVATA and WUSCHEL genes. Cell 100, 635-644.

Semler B. L. et al. (1982) A membrane-associated precursor to poliovirus VPg identified by immunoprecipitation with antibodies directed against a synthetic heptapeptide. Cell 28, 405-412.

Shioda T. et al. (2000) A green fluorescent protein-reporter mammalian two-hybrid system with extrachromosomal maintenance of a prey expression plasmid: Application to interaction screening. Proc. Natl. Acad. Sci. U.S.A 97, 5220-5224.

Soni R. et al. (1995) A family of cyclin D homologs from plants differentially controlled by growth regulators and containing the conserved retinoblastoma protein interaction motif. Plant Cell 7, 85-103.

Sorrell D. A. et al. (1999) Distinct cyclin D genes show mitotic accumulation or constant levels of transcripts in tobacco bright yellow-2 cells. Plant Physiol 119, 343-352.

Sugita K. et al. (2000) Technical advance: A transformation vector for the production of marker free transgenic plants containing a single copy transgene at high frequency [In Process Citation]. Plant J. 22, 461-469.

Sun Y. et al. (1999) Characterization of maize (Zea mays L.) Wee1 and its activity in developing endosperm. Proc. Natl. Acad. Sci. U.S.A 96,4180-4185.

Suzuki H., Fowler T. J., & Tierney M. L. (1993) Deletion analysis and localization of SbPRP1, a soybean cell wall protein gene, in roots of transgenic tobacco and cowpea. Plant Mol. Biol. 21, 109-119.

Swenson K. I., Farrell K. M., & Ruderman J. V. (1986) The clam embryo protein cyclin A Induces entry into M phase and the resumption of meiosis in Xenopus oocytes. Cell 47, 861-870.

Tamura R. N. et al. (1991) Cell type-specific integrin variants with alternative alpha chain cytoplasmic domains. Proc. Natl. Acad. Sci. U.S.A 88, 10183-10187.

Torii K. U. et al. (1996) The Arabidopsis ERECTA gene encodes a putative receptor protein kinase with extracellular leucine-rich repeats. Plant Cell 8, 735-746.

Trieu A. T. et al. (2000) Technical Advance: Transformation of Medicago truncatula via infiltration of seedlings or flowering plants with Agrobacterium. Plant J. 22, 531-541.

Uribe X. et al. (1998) Maize alpha-tubulin genes are expressed according to specific patterns of cell differentiation. Plant Mol. Biol. 37, 1069-1078.

van den Berg C. et al. (1995) Cell fate in the Arabidopsis root meristem determined by directional signalling: Nature 378, 62-65.

van den Berg C. et al. (1997) Short-range control of cell differentiation in the Arabidopsis root meristem. Nature 390, 287-289.

van der Zaal E. J. et al. (1991) Promoters of auxin-induced genes from tobacco can lead to auxin inducible and root tip-specific expression. Plant Mol. Biol. 16, 983-998.

van Haaren M. J. et al. (1987) Overdrive is a T-region transfer enhancer which stimulates T-strand production in Agrobacterium tumefaciens. Nucleic Acids Res. 15, 8983-8997.

Van Sluys M. A., Tempe J., & Fedoroff N. (1987) Studies on the introduction and mobility of the maize Activator element in Arabidopsis thaliana and Daucus carota. EMBO J. 6, 3881-3889.

Vidmar J. J. et al. (2000) Cloning and characterization of a root specific high-affinity sulfate transporter from arabidopsis thaliana(1) [In Process Citation]. FEBS Lett. 475, 65-69.

Wanapu C. & Shinmyo A. (1996) cis-regulatory elements of the peroxidase gene in Arabidopsis thaliana involved in root-specific expression and responsiveness to high-salt stress. Ann. N.Y. Acad. Sci. 782,107-114.

Wang H., Fowke L. C., & Crosby W. L. (1997) A plant cyclin-dependent kinase inhibitor gene [letter]. Nature 386, 451-452.

Wang K. et al. (1987) Sequence context of the T-DNA border repeat element determines its relative activity during T-DNA transfer to plant cells. Mol. Gen. Genet. 210, 338-346.

Webb K. J. et al. (2000) Mesorhizobium loti increases root-specific expression of a calcium binding protein homologue identified by promoter tagging in Lotus japonicus [In Process Citation]. Mol. Plant Microbe Interact. 13, 606-616.

Wilson A. et al. (1999) Evidence for the activation of a MAP kinase upon phosphate-induced cell cycle re-entry in tobacco cells. Physiol. Plant. 102, 532-538.

Winicov I. & Bastola D. R. (1999) Transgenic overexpression of the transcription factor alfin1 enhances expression of the endogenous MsPRP2 gene in alfalfa and improves salinity tolerance of the plants. Plant Physiol 120, 473-480.

Winicov I. (2000) Alfin1 transcription factor overexpression enhances plant root growth under normal and saline conditions and improves salt tolerance in alfalfa. Planta 210, 416-422.

Wodak S. J. (1987) Computer-aided design in protein engineering. Ann. N.Y. Acad. Sci. 501,1-13.

Woo H. H. & Hawes M. C. (1997) Cloning of genes whose expression is correlated with mitosis and localized in dividing cells in root caps of Pisum sativum L. Plant Mol. Biol. 35,1045-1051.

Woulfe J. et al. (1994) Post-translational processing of the neurotensin/neuromedin N precursor in the central nervous system of the rat—II. Immunohistochemical localization of maturation products. Neuroscience 60, 167-181.

Xie Q. et al. (1996) Plant cells contain a novel member of the retinoblastoma family of growth regulatory proteins. EMBO J. 15, 4900-4908.

Xu Y. et al. (1995) Characterization of a rice gene family encoding root-specific proteins [published erratum appears in Plant Mol Biol 1995 March;27(5):1060]. Plant Mol. Biol. 27, 237-248.

Yamamoto E., Karakaya H. C., & Knap H. T. (2000) Molecular characterization of two soybean homologs of Arabidopsis thaliana CLAVATA1 from the wild type and fasciation mutant. Biochim. Biophys. Acta 1491, 333-340.

Yamamoto Y. T. et al. (1991) Characterization of cis-acting sequences regulating root-specific gene expression in tobacco. Plant Cell 3, 371-382.

Zeng Y. et al. (1998) Replication checkpoint requires phosphorylation of the phosphatase Cdc25 by Cds1 or Chk1. Nature 395, 507-510.

Zhang Y. L. et al. (1996) A superactive peptidomimetic analog of a farnesylated dodecapeptide yeast pheromone. Biochem. Biophys. Res. Commun. 224, 327-331.

1. An isolated nucleic acid comprising a transcriptional regulatory root promoter or a functional fragment of such a promoter and/or comprising a nucleic acid sequence encoding a novel LRR receptor-like kinase (protein) or an immunologically active and/or functional fragment of such a protein (kinase) selected from the group consisting of:
(a) a nucleic acid comprising the DNA sequence as given in any of SEQ ID NOs 1, 2, 3 or 18, or the complement thereof,
(b) a nucleic acid comprising the RNA sequences corresponding to any of SEQ ID NOs 1, 2, 3 or 18, or the complement thereof,
(c) a nucleic acid specifically hybridizing with the nucleotide sequence as defined in (a) or (b),
(d) a nucleic acid encoding a protein comprising the amino acid sequence as given in SEQ ID NO 4, SEQ ID NO 5 or SEQ ID NO 19,
(e) a nucleic acid which is degenerated, as a result of the genetic code, to a nucleotide sequence as defined in any of (a) to (d), and which sequence codes for a protein having the amino acid sequence as given in SEQ ID NO 4, SEQ ID NO 19,
(f) a nucleic acid which is diverging, due to the differences in codon usage between the organisms, to a nucleotide sequence as defined in any of (a) to (d), and which sequence codes for a protein having the amino acid sequence as given in SEQ ID NO 4, SEQ ID NO 19,
(g) a nucleic acid which is diverging, due to the differences between alleles, to a nucleotide sequence as defined in any of (a) to (d), and which sequence codes for a protein having the amino acid sequence as given in SEQ ID NO 4, SEQ ID NO 19,
(h) a nucleic acid encoding a fragment of a protein encoded by a DNA sequence as given in SEQ ID NO 1 or SEQ ID NO 3 or encoding a fragment of a protein encoded by a nucleic acid as defined in any one of (a) to (g), wherein said fragment comprises the sequence as represented in SEQ ID NO 5, and,
(k) a nucleic acid encoding a protein as defined in SEQ ID NO 4, SEQ ID NO 19 or a nucleic acid as defined in any one of (a) to (h), said nucleic acid interrupted by intervening DNA,
provided that said nucleic acid is not one of the nucleic acids as deposited under the GenBank Accession numbers ABO17061, AV538507, AV520681 or AQ966419.

2. An isolated nucleic acid comprising a transcriptional regulatory root promoter comprising the sequence as given in any of SEQ ID NOs 1, 2 or 18.

3. An isolated nucleic acid comprising a transcriptional regulatory root promoter consisting of the sequence as given in SEQ ID NO 2 or 18, or a functional part of said sequence which is able to regulate gene expression in a root specific manner, provided that said nucleic acid is not one of the nucleic acids as deposited under the GenBank Accession numbers AV538507 or AV520681.

4. An isolated nucleic acid comprising a transcriptional regulatory root specific promoter comprising:
(a) at least a functional part of the DNA sequence as given in any of SEQ ID NO 1, 2 or 18, and,
(b) a second transcriptional regulatory sequence, optionally a regulatory promoter sequence not normally exhibiting root-specificity.

5. An isolated nucleic acid according to claim 4 wherein said second transcriptional regulatory promoter sequence is the ubiquitin promoter.

6. An isolated nucleic acid selected from the group consisting of:
(a) a nucleic acid consisting of at least part of the DNA sequence as given in SEQ ID NO 1 or 3, or the complement thereof,
(b) a nucleic acid encoding a protein as given in SEQ ID NO 4 or 19 or encoding a fragment of said protein, wherein said fragment comprises the sequence as represented in SEQ ID NO 5, and
(c) a nucleic acid encoding a protein with an amino acid sequence which is at least 65% identical to the protein as given in SEQ ID NO 4 or 9, wherein said amino acid sequence comprises the sequence as represented in SEQ ID NO 5, characterised in that said nucleic acid encodes a novel LRR receptor-like kinase protein or an immunologically active and/or functional fragment of such a protein, and further provided that said nucleic acid is not one of the nucleic acids as deposited under the GenBank Accession numbers ABO17061 or AQ966419.

7. An isolated nucleic acid according to any of claims 1 to 6 which is DNA, cDNA, genomic DNA, synthetic DNA, or RNA wherein T is replaced by U.

8. A nucleic acid molecule of at least 15 nucleotides in length hybridizing specifically with a nucleic acid of any of claims 2 to 7.

9. A nucleic acid molecule of at least 15 nucleotides in length specifically amplifying a nucleic acid of any of claims 2 to 7.

10. A vector comprising a nucleic acid sequence according to any of claims 1 to 7.

11. A vector according to claim 10 which is an expression vector wherein said nucleic acid sequence encoding a novel LRR receptor-like kinase or an immunologically active and/or functional fragment thereof, is operably linked to one or more control sequences allowing the expression in prokaryotic and/or eukaryotic host cells.

12. A vector according to claim 10 wherein said vector comprises at least part of a nucleic acid according to any of claims 2 to 7 and wherein said transcriptional regulatory root promoter or functional parts thereof is operably linked to one or more genes of interest.

13. A host cell containing a nucleic acid molecule according to any of claim 1 to 7 or a vector according to any of claims 10 to 12.

14. The host cell according to claim 13, wherein said host cell is a bacterial, insect, fungal, plant or animal cell.

15. An isolated LRR receptor-like kinase comprising one of the polypeptides selected from the group consisting of:
(a) a polypeptide as given in SEQ ID NO 4 or SEQ ID NO 19,
(b) a polypeptide comprising the amino acid sequence as given in SEQ ID NO 5, and,
(c) a polypeptide encoded by a nucleic acid as given in SEQ ID NO 1 or 3,
or a homologue or a derivative of said protein, or an immunologically active and/or functional fragment thereof.

16. A polypeptide as defined in claim 15 being a root CLAVATA homologue or a functional homologue thereof

17. An isolated polypeptide comprising the sequence represented in SEQ ID NO 5 encodable by a nucleic acid of any of claims 1 to 7, or a homologue or a derivative thereof, or an immunologically active and/or functional fragment thereof.

18. A method for producing a polypeptide according to any of claims 15 to 17 comprising culturing a host cell of claim 13 or 14 under conditions allowing the expression of the polypeptide and recovering the produced polypeptide from the culture.

19. An antibody specifically recognizing an LRR receptor-like kinase (protein) of any of claims 15 to 17 or recognizing immunologically active parts or specific epitopes thereof.

20. A method for the production of transgenic plants, plant cells or plant tissues comprising the introduction of a nucleic acid molecule according to any of claims 1 to 7 in an expressible format or a vector according to any of claims 10 to 12 in said plant, plant cell or plant tissue.

21. The method of claim 20 further comprising regenerating a plant from said plant cell.

22. A transgenic plant cell comprising a nucleic acid sequence of any of claims 1 to 7 which is operably linked to regulatory elements allowing transcription and/or expression of said nucleic acid in plant cells or obtainable by a method of claim 20 or 21.

23. The transgenic plant cell of claim 22 wherein said nucleic acid of any of claims 1 to 7 is stably integrated into the genome of said plant cell.

24. A transgenic plant or plant tissue comprising plant cells of claim 22 or 23.

25. A harvestable part of a plant of claim 24.

26. The harvestable part of claim 25 which is selected from the group consisting of seeds, leaves, fruits, stem cultures, rhizomes, tubers and bulbs.

27. The progeny derived from any of the plants or plant parts of any of claims 24 to 26.

28. A method for conferring root-specificity, root-meristem-specificity, root-vascular-tissue-specificity, root-endodermis-specificity, root-cortex-specificity, root-epidermis-specificity, root-quiescent-center-specificity, and/or abundant expression in root and/or early expression in root to other promoter sequences comprising the fusion at least a functional part of the DNA sequence as given in SEQ ID NO 1, 2 or 18 to a second transcriptional regulatory promoter sequence normally not exhibiting root-specificity.

29. A method for root-specific expression of a gene(s) of interest comprising operably linking of said gene(s) of interest to a transcriptional regulatory root-specific promoter as defined in any of claims 2 to 5.

30. A method for modifying cell fate and/or plant development and/or plant morphology and/or plant biochemistry and/or plant physiology comprising the modification of expression in the meristem of the main and the lateral roots, in the vascular tissue of the root or in the lateral root primordia of a gene(s) of interest operably linked to a transcriptional regulatory root-specific promoter as defined in any of claims 2 to 5.

31. A method for phytoremediation comprising the expression of a gene(s) of Interest under the control of a transcriptional regulatory root-specific promoter as defined in any of claims 2 to 5.

32. A method for environmental remediation comprising the expression of a gene(s) of interest under the control of a transcriptional regulatory root-specific promoter as defined in any of claims 2 to 5.

33. A method for enhancing plant growth and crop yield comprising the expression of a gene(s) of interest under the control of a a transcriptional regulatory root-specific promoter as defined in any of claims 2 to 5.

34. A method for conferring enhanced resistance to pathogens which attack the belowground plant tissue comprising the expression of a gene(s) of interest under the control of a transcriptional regulatory root-specific promoter as defined in any of claims 2 to 5.

35. A method for enhancing freezing tolerance in plants comprising the expression of a gene(s) of interest under the control of a transcriptional regulatory root-specific promoter as defined in any of claims 2 to 5.

36. A method for enhancing resistance of a plant to drought and/or high salt comprising the expression of a gene(s) of interest under the control of a transcriptional regulatory root-specific promoter as defined in any of claims 2 to 5.

37. A method for the production of light-sensitive proteins comprising the expression of a gene encoding said light-sensitive protein under the control of a transcriptional regulatory root-specific promoter as defined in any of claims 2 to 5.

38. A method for root specific gene silencing comprising the expression of an antisense nucleotide sequence to the gene of interest, a gene silencing construct or a ribozyme under the control of a transcriptional regulatory root-specific promoter as defined in any of claims 2 to 5.

39. A method for stimulating root meristem formation comprising the expression of a gene that influences the proliferation and/or differentiation of the root meristem under the control of a transcriptional regulatory root-specific promoter as defined in any of claims 2 to 5.

40. A method for stimulating root meristem formation comprising the expression in particular cells, tissues or organs of a plant, of a nucleic acid encoding an LRR receptor-like kinase operably linked to a plant-operable promoter sequence.

41. A method stimulating root meristem formation comprising the expression in particular cells, tissues or organs of a plant, of a nucleic acid encoding an LRR receptor-like kinase as defined in any of claims 15 to 17 operably linked to a plant-operable promoter sequence.

42. A method stimulating root meristem formation comprising the expression in particular cells, tissues or organs of a plant, of a nucleic acid encoding an LRR receptor-like kinase under the control of a transcriptional regulatory root promoter as defined in any of claims 2 to 5.

43. A method for stimulating root meristem formation comprising the expression in particular cells, tissues or organs of a plant, of a nucleic acid encoding a polypeptide as defined in any of claims 15 to 17 under the control of a transcriptional regulatory root promoter as defined in any of claims 2 to 5.

44. A method for root-meristem maintenance comprising the expression of a gene that influences the proliferation and/or differentiation of the root meristem under the control of a transcriptional regulatory root-specific promoter as defined in any of claims 2 to 5.

45. A method for root-meristem maintenance comprising the expression in particular cells, tissues or organs of a plant, of a nucleic acid encoding an LRR receptor-like kinase operably linked to a plant-operable promoter sequence.

46. A method for root-meristem maintenance comprising the expression in particular cells, tissues or organs of a plant, of a nucleic acid encoding an LRR receptor-like kinase as defined in any of claims 15 to 17 operably linked to a plant-operable promoter.

47. A method for root-meristem maintenance comprising the expression in particular cells, tissues or organs of a plant, of a nucleic acid encoding an LRR receptor-like kinase under the control of transcriptional regulatory root promoter as defined in any of claims 2 to 5.

48. A method for root-meristem maintenance comprising the expression in particular cells, tissues or organs of a plant, of a nucleic acid encoding a polypeptide as defined in any of claims 15 to 17 under the control of a transcriptional regulatory root promoter as defined in any of claims 2 to 5.

49. A method for enhancing root formation and/or root growth comprising the expression in particular cells, tissues or organs of a plant, of a nucleic acid encoding an LRR receptor-like kinase operably linked to a plant-operable promoter sequence.

50. A method for enhancing root formation and/or root growth comprising the expression in particular cells, tissues or organs of a plant, of a nucleic acid encoding an LRR receptor-like kinase as defined in any of claims 15 to 17 under the control of a plant operable promoter sequence.

51. A method for enhancing root formation and/or root growth comprising the expression in particular cells, tissues or organs of a plant, of a nucleic acid encoding an LRR receptor-like kinase under the control of a transcriptional regulatory root promoter as defined in any of claims 2 to 5.

52. A method for enhancing root-formation and/or root growth comprising the expression in particular cells, tissues or organs of a plant, of a nucleic acid encoding a polypeptide as defined in any of claims 15 to 17 under the control of a transcriptional regulatory root promoter as defined in any of claims 2 to 5.

53. A method for enhancing overall growth and yield comprising the expression in particular cells, tissues or organs of a plant, of a nucleic acid encoding an LRR receptor-like kinase operably linked to a plant-operable promoter sequence.

54. A method for enhancing overall growth and yield comprising the expression in particular cells, tissues or organs of a plant, of a nucleic acid encoding an LRR receptor-like kinase as defined in any of claims 15 to 17 operably linked to a plant operable promoter sequence.

55. A method for enhancing overall growth and yield comprising the expression in particular cells, tissues or organs of a plant, of a nucleic acid encoding an LRR receptor-like kinase under the control of a transcriptional regulatory root promoter as defined in any of claims 2 to 5.

56. A method for enhancing overall growth and yield comprising the expression in particular cells, tissues or organs of a plant, of a nucleic acid encoding a polypeptide as defined in any of claims 15 to 17 under the control of a transcriptional regulatory root promoter as defined in any of claims 2 to 5.

57. A method for modifying cell fate and/or plant development and/or plant morphology and/or plant biochemistry and/or plant physiology comprising the modification of expression in particular cells, tissues or organs of a plant, of a nucleic acid encoding an LRR receptor-like kinase operably linked to a plant-operable promoter sequence.

58. A method for modifying cell fate and/or plant development and/or plant morphology and/or plant biochemistry and/or plant physiology comprising the modification of expression in particular cells, tissues or organs of a plant, of a nucleic acid encoding an LRR receptor-like kinase as defined in any of claims 15 to 17 operably linked to a plant-operable promoter sequence.

59. A method for modifying cell fate and/or plant development and/or plant morphology and/or plant biochemistry and/or plant physiology comprising the modification of expression in particular cells, tissues or organs of a plant, of a nucleic acid encoding an LRR receptor-like kinase under the control of a transcriptional regulatory root promoter as defined in any of claims 2 to 5.

60. A method for modifying cell fate and/or plant development and/or plant morphology and/or plant biochemistry and/or plant physiology comprising the modification of expression in particular cells, tissues or organs of a plant, of a nucleic acid encoding a polypeptide as defined in any of claims 15 to 17 under the control of a transcriptional regulatory root promoter as defined in any of claims 2 to 5.

61. A method to confer pathogen resistance to a plant comprising the expression in particular cells, tissues or organs of a plant, of a nucleic acid encoding an LRR receptor-like kinase operably linked to a plant-operable promoter sequence.

62. A method to confer pathogen resistance to a plant comprising the expression in particular cells, tissues or organs of a plant, of a nucleic acid encoding an LRR receptor-like kinase as defined in any of claims 15 to 17 operably linked to a plant-operable promoter sequence.

63. A method to confer pathogen resistance to a plant comprising the expression in particular cells, tissues or organs of a plant, of a nucleic acid encoding an LRR receptor-like kinase under the control of a transcriptional regulatory root promoter as defined in any of claims 2 to 5.

64. A method to confer pathogen resistance to a plant comprising the expression in particular cells, tissues or organs of a plant, of a nucleic acid encoding a polypeptide as defined in any of claims 15 to 17 under the control of a transcriptional regulatory root promoter as defined in any of claims 2 to 5.

65. A method for identifying and obtaining proteins interacting with a (LRR receptor-like kinase) polypeptide of any of claims 15 to 17 comprising a screening assay wherein a polypeptide of any of claims 15 to 17 is used or expressed.

66. A method according to claim 65 comprising a two-hybrid screening system wherein a nucleic acid encoding a polypeptide of any of claims 15 to 17 as a bait and a cDNA library as prey are expressed.

67. A method for modulating the interaction between an LRR receptor-like kinase (protein) as defined in any of claims 15 to 17 and interacting proteins obtainable by a method according to claim 65 or 66.

68. A method for identifying and obtaining compounds interacting with an LRR receptor-like kinase (protein) comprising the steps of:
(a) providing a two-hybrid screening system wherein a polypeptide of any of claims 15 to 17 and a protein interacting with said LRR receptor-like kinase (protein) or an interacting protein obtainable by a method of claim 65 or 66 are expressed,
(b) interacting said compound with the complex formed by the expressed proteins as defined in a),
(c) detecting a second complex, wherein the presence of said second complex identifies a compound which specifically binds to one of said polypeptides or said second complex, and
(d) identifying the compound.

69. A method for identifying compounds or mixtures of compounds which specifically bind to a polypeptide of any of claims 15 to 17, comprising:
(a) combining a polypeptide of any of claims 15 to 17 with said compound or mixtures of compounds under conditions suitable to allow complex formation, and,
(b) detecting complex formation, wherein the presence of a complex identifies a molecule which specifically binds said polypeptide.

70. Use of a molecule identifiable by means of a method of claim 68 or 69 as a plant growth regulator or herbicide.

71. A method for the production of a plant growth regulator or herbicide composition comprising the steps of the method of claim 68 or 69 and formulating the compounds obtained from said steps in a suitable form for the application in agriculture or plant cell or tissue culture

72. Use of a nucleic acid molecule of any of claims 1 to 7, a vector of claim 10 to 12, a polypeptide of claim 15 to 17 or an antibody of claim 19 for modifying cell fate and/or plant development and/or plant morphology and/or plant biochemistry and/or plant physiology.

73. Diagnostic composition comprising at least one nucleic acid molecule of any of claims 1 to 7, vector of claim 10 to 12, polypeptide of claim 15 to 17 or antibody of claim 19.


SEQUENCE LISTING (19 sequences)

SEQ ID NO: 1 - DNA, 7438 bp, Arabidopsis thaliana
ctcgagtttcagatgtttctattaaataagtaatgaaagcagctctaattctcccaaaattcacaagtatagaaagatagaaagatagaaacagagttattgcatggaagctgaaaatcatacagcatggtggatataaccaatgagacacaaaacaatcacggcatgtgttatagaacaacacttatcatctgtattaacataaagcaaaagttcctatacgacatagtccaaagttacagaccataagcacaccttatgatttgttgtaccctctaaaacacaggatgatgacaagaagcaaatttgcccaaatttactgagggaaaaacgagggcttaacgctctcatatctcaaacaaaaacttctcaaacagtttcatgtgttcagattggagggcaatggcaacggataagctgccatcattagtaggacttggtagcacaaaagacaaaccctcgtatggaattccaccaggtcccataaagataggacgaccccaaccaaagtctgcatcataaataggtaatctaacccagcttgtgattcccaaatttgggcacttgtaggtatgtgcaccgcggacaagggctgacagatcaggctgcatctccaggtagtcaagagctgacctcagatagttatcatccatacgaaccaagaaatcatgaatctgtcctgcagcataccatgttggcttagataacaaatcccctgcaacagccaatggtgttgcagtgaatatcacattcccaaagtaaccaggaggcagctgcggacgtagtctagaccttccatcagttgcaatgtacagtttcgtctcttggtcgtttggaagccctcgcgcctttcccactgatctccacacatgccctgccaacatctcgtatgagctgtagctgacagtgttcccatcctccttggatttcgccttaagagcaacaagctggtctcgtgttaatttgaatatagagacagtggtattctcaggtcctgatttagacggatcaagaggtatcttcatacttggtgcaggctgatattcaacatgatgaaaagcaggctgtggcgggtccctagctcggaggagtgttcgatcaatgaaaggtggaatggttaggtcaagaccacgagccatatcagaccatgtgttgataaaatgaagaccagagaaaccatctgccgcgtgatgttgcatcccaaccccaagtgaagctcccccacatttaaagaaagtcacctatatcatcaaacaatatcattacataactaaaaacagaacatacatcatcaaagaatatcattacagaactaaaaaacagaacaacacaagcattcaataaattaaaaaagggaaagactttaaattaagctgacctgcaaaacgagaagcgggaaagagtgaatgccagcggagtgatcaacttcgggaataagctgacggagattaagggtaggagcaaaatcaccaaaatcatcgataacagaaggagtatcagccacaacgaagagaacaccagcaccgttacaatcgatctcaatacgaccatcatcgtctctcttcaagcgaccagccatagggtaaaaagggacaagggctttggaaagagcttccttcatgacctgagggtcaaagaaattggaagcgccggtgggtctgtagaagtagacactaggggtatggaatctggggatgacaaggtcgacgttggagttccaaagattagtgattggtgtctcggtggcaggccggaccatggtggaatctctgatgttaattttcatatttttaagaaagatcaaacgaacaaacaaattttgttttgagtttgatctgagaaattgtgtgagaagcgaggaggggtagatataagtgggtgtgaacaaaaaaggaaaggtcgtaatccttcacctaccgccccacgaccccgcctactccatctgtcgaccaactctctcagaaggtttatcattggtccatctcatcactcggtcctacttttctatccggggttggtaaacccgttccctctgtcctaaggcccaaataatactaatgggcttaaatagtataaagttcgaatgaagaagaaggaaaaaaaattgacaagttgattgttatagctaccgtacaagttagtcaatcgaatgccaaagtttggcttaacttaactcattaccacgtagcagagcataacaacaaccaccaataacatctaacggcgtaaactcacgtgttaagagaattacgtcagaatccaagaaagtctttttttcagtacaccacaaataaattatgttgaatttatatccgaactatactatattatatgaaaacatacctcaattagtcataaatttcacatttcattttaatttattaactaatggttgttgttgcggatagcttttctttgctttctgttctaaaattatttcttagccattatttgatgatccaacttggtttgaggatctaataaaaattcaatttctttgaagcattcattggcttaatgatccaaaccttttgtcctcccactctctaagaaaataaagcaaaagcaaaactaaaagaaaagcaactagtaccgtgggttggtcaatggtaacccatgctaccaaataccaatcaaagcttaaatgacaatttagtaccttgggttggtcatgatttagagcggaacaaatataccatacatcaaacgaggatatacagagaaaattcatggaagtatggaatttagaggacaatttctcttctgggctacaacggaccggcccattcgctcatttacccagaggtatcgagtttgtggacttttgatgccgctagagactattggcatcggattgaaaaaaatgtttacttcgttgttaacaattttctgaatgcaatattttccttgtcatgaatatttaaacttgttattactttcttttagcttaggtgtggacaattatggagtttacttcaaacgaggaagaatcttaaacgctcggttcaggtctcgaaaacaaaccaactcacaatcctgacttaattgaggaaaacaatgcaaaaccacatgcatgcttccatatttctatcataatcttataagaaaaaacactactaagtgaaatgattctgtatatatataaccaatgccttttgttttgtgatattttatgtatatataactattgacttttgtcatctatggatagtgtctcgggctcttggcaaacatatttcaaagaaaagttaatgactgtaattaattaatctgaagctagaaacagaaccccgaggtaaaagaaaaagacagagcacatgaagtttagtacttttatatatttaatatatcattctttcttattgcttatctctaaagcaaaaacttccctaaaccctaagccaaaggactcagatcgatgcagaaccaagaaggcttgttttggatttgagagccaaatgcaaagaaaaaaactcttatgtctttgcaaatgccgattccaagaaaaaaagccttaactgtttctcatttttccattacattgtctctgtttttagctttcttcatctcctcgacctctgcatcaaccaatgaagtctcagctttgatttcttggcttcacagctcgaactcgccgccaccgtcagtcttctccggctggaatccttctgattctgacccatgtcaatggccttacattacttgttcctcctcagacaacaaactcgttacagagatcaatgtcgtctctgttcagttagctttacctttccctcctaacatttcttcgtttacttcacttcagaaactcgttatctccaacactaatctcaccggagctatatcttctgagatcggagattgttcggagcttatagttatcgatttaagctcaaatagtcttgttggtgaaataccttcgagtctagggaagctcaagaatcttcaagaactttgtttaaactccaatggcctcacaggaaagatcccaccggaactcggcgactgcgttagcctcaagaatcttgagattttcgataactacttatccgagaatctcccgttggagctcggaaagatctcgactcttgagagcataagagccggaggaaactcagagctttcagggaagatcccggaggagatcggaaactgtaggaatctcaaggtcttagggttagcagcaacgaaaatatccggttctttacctgtttcgttgggtcaactaagcaagctccagtctttatctgtctattctacaatgctctcaggtgagatccctaaagagcttggaaactgctctgaacttatcaatctgtttctatacgacaatgacttgtccggcacgcttccaaaagaactaggaaagcttcaaaacctagagaagatgcttctatggcagaataatctccacggacctatccctgaggagattggattcatgaaaagcttaaacgccattgatctctctatgaactatttctcaggaaccatccctaaatcgttcggtaacttgtcgaatctccaagagcttatgcttagcagcaacaacatcaccgggtcaataccttcgattctaagtaactgcacaaagcttgttcagttccagattgacgccaatcagatttcaggtttgattccaccagagattggattgcttaaggagctcaacatcttcttgggatggcagaataagctagaagggaatattccggacgagttagctggttgtcagaatcttcaagctcttgacttgtcacagaattatctcaccggatcattacctgcaggtttgtttcagcttcgtaatctgactaagctcttgcttatatctaatgccatctccggtgttatcccactggagatcgggaactgcacttcgcttgtcagattaaggcttgtcaataacagaatcaccggagaaatccctaaaggtattggatttcttcagaaccttagcttcctcgacttgtctgagaacaatctctccggtccagttcctttggagataagtaattgtagacaactccagatgcttaacttgagcaacaacactcttcaaggttatcttcctctgtctttatcatcgttaacaaagcttcaagtacttgatgtctcttccaatgacttgactggcaagatacctgatagtctaggtcatctcatttcactcaaccggctcattctaagtaagaattctttcaatggagaaatcccttcgtctctcggtcactgcacgaatcttcagcttcttgatctcagcagtaataacatctctggaactataccggaggagctttttgacatccaagatctagacattgcgttaaacttgagctggaattcattagatggctttatcccggaaaggatttccgcgcttaaccgattatccgtgctcgatatttcgcataacatgctttcaggcgacctttctgcgctgtccggtctagaaaacttggtttctctgaacatctctcataacagattctcaggttatcttccagatagtaaagtgtttagacagctgataggagcagagatggaaggaaacaatggactctgttccaaaggtttcaggtcttgctttgtaagtaacagttcacagttaactacacagcgtggtgtgcactcacatagactcaggatagccattggattgctaatcagcgtgacagcggttctagcggtattaggcgtgttggcggttatacgagctaaacaaatgattcgagatgataacgattcagagactggagaaaatctatggacatggcaattcacaccttttcagaaactcaacttcacagtcgaacatgtactcaagtgtttggtagaaggtaatgttatagggaaaggttgctccgggatagtgtacaaagctgaaatgcctaacagagaagtcatcgcggtgaaaaagctctggccagtgacagtacctaatctgaatgagaaaactaagtcatcaggagttcgagactcattctcagctgaagtaaaaacacttggatcgatcagacacaagaacattgtacgcttcttgggatgttgttggaacaagaacactagacttcttatgtatgattatatgtcaaatgggagtttgggaagtttgcttcacgaaaggagcggtgtatgtagcttaggatgggaagtgaggtacaagattatacttggtgcagctcagggtttggcttacttgcaccatgactgtgttcctcccattgttcatagagacatcaaggcaaataatattctgattggccctgattttgaaccttacattggagatttcggactcgctaagcttgttgatgatggcgactttgctcgttcttccaacaccattgctggttcctatggttacatagctccaggtacttcccaaatttctgttttgctgctaattagttaacttgatacatcataagttactagtagtcttagcctagtaatggtagaaattcaggtttatagtttggttttggtttttggcattttgaaaagaatagtattttggttcagttgattttcgatttggtttaaaattttagaaaaaaaatggtagaagaaataatttttgattaatattatatcatctactgtatacgatttaaaaaaaaaattgatttggtttggtctcgatattttgcttgtagcttggttcaatatttttactatatatatgattcatgactttcatactaatcaatatttgttcggtttcgttttgattagtctgaaagtgaatcatatagttaaaatattgaccaatctatcatatacttacacgctacaagattttcgatgtaattttggtgtaagcattttggtttttatatgttcatccttagtaactaacaacaaatatcaattgggaagcataacgttatatattcccataaggctataacactataattatgcagaatacggatactcaatgaagataacagagaaaagcgacgtgtacagctatggagtcgtagtgctcgaggtactaacgggtaagcaaccaatcgatccaacgataccagatggactccacatagtggactgggtcaagaaaatcagagacatacaagtaatcgaccaaggactacaagcaagaccagagtcagaggttgaagagatgatgcaaacgctaggagtcgcgcttttatgcattaatccaatacccgaagacaggcctacaatgaaagatgtggctgctatgcttagtgagatatgtcaagagagagaggaatcgatgaaagttgatggttgctcgggaagttgtaacaatggaagagaacgtggcaaagatgattcgacttcatcggttatgcaacaaacggctaagtatttgagaagtagtagcacgagtttctctgcgtcttctttgctttactcttcttcttcttctgctacttctaatgttagaccaaatcttaaataa

SEQ ID NO: 2 - DNA, 3499 bp, Arabidopsis thaliana
ctcgagtttcagatgtttctattaaataagtaatgaaagcagctctaattctcccaaaattcacaagtatagaaagatagaaagatagaaacagagttattgcatggaagctgaaaatcatacagcatggtggatataaccaatgagacacaaaacaatcacggcatgtgttatagaacaacacttatcatctgtattaacataaagcaaaagttcctatacgacatagtccaaagttacagaccataagcacaccttatgatttgttgtaccctctaaaacacaggatgatgacaagaagcaaatttgcccaaatttactgagggaaaaacgagggcttaacgctctcatatctcaaacaaaaacttctcaaacagtttcatgtgttcagattggagggcaatggcaacggataagctgccatcattagtaggacttggtagcacaaaagacaaaccctcgtatggaattccaccaggtcccataaagataggacgaccccaaccaaagtctgcatcataaataggtaatctaacccagcttgtgattcccaaatttgggcacttgtaggtatgtgcaccgcggacaagggctgacagatcaggctgcatctccaggtagtcaagagctgacctcagatagttatcatccatacgaaccaagaaatcatgaatctgtcctgcagcataccatgttggcttagataacaaatcccctgcaacagccaatggtgttgcagtgaatatcacattcccaaagtaaccaggaggcagctgcggacgtagtctagaccttccatcagttgcaatgtacagtttcgtctcttggtcgtttggaagccctcgcgcctttcccactgatctccacacatgccctgccaacatctcgtatgagctgtagctgacagtgttcccatcctccttggatttcgccttaagagcaacaagctggtctcgtgttaatttgaatatagagacagtggtattctcaggtcctgatttagacggatcaagaggtatcttcatacttggtgcaggctgatattcaacatgatgaaaagcaggctgtggcgggtccctagctcggaggagtgttcgatcaatgaaaggtggaatggttaggtcaagaccacgagccatatcagaccatgtgttgataaaatgaagaccagagaaaccatctgccgcgtgatgttgcatcccaaccccaagtgaagctcccccacatttaaagaaagtcacctatatcatcaaacaatatcattacataactaaaaacagaacatacatcatcaaagaatatcattacagaactaaaaaacagaacaacacaagcattcaataaattaaaaaagggaaagactttaaattaagctgacctgcaaaacgagaagcgggaaagagtgaatgccagcggagtgatcaacttcgggaataagctgacggagattaagggtaggagcaaaatcaccaaaatcatcgataacagaaggagtatcagccacaacgaagagaacaccagcaccgttacaatcgatctcaatacgaccatcatcgtctctcttcaagcgaccagccatagggtaaaaagggacaagggctttggaaagagcttccttcatgacctgagggtcaaagaaattggaagcgccggtgggtctgtagaagtagacactaggggtatggaatctggggatgacaaggtcgacgttggagttccaaagattagtgattggtgtctcggtggcaggccggaccatggtggaatctctgatgttaattttcatatttttaagaaagatcaaacgaacaaacaaattttgttttgagtttgatctgagaaattgtgtgagaagcgaggaggggtagatataagtgggtgtgaacaaaaaaggaaaggtcgtaatccttcacctaccgccccacgaccccgcctactccatctgtcgaccaactctctcagaaggtttatcattggtccatctcatcactcggtcctacttttctatccggggttggtaaacccgttccctctgtcctaaggcccaaataatactaatgggcttaaatagtataaagttcgaatgaagaagaaggaaaaaaaattgacaagttgattgttatagctaccgtacaagttagtcaatcgaatgccaaagtttggcttaacttaactcattaccacgtagcagagcataacaacaaccaccaataacatctaacggcgtaaactcacgtgttaagagaattacgtcagaatccaagaaagtctttttttcagtacaccacaaataaattatgttgaatttatatccgaactatactatattatatgaaaacatacctcaattagtcataaatttcacatttcattttaatttattaactaatggttgttgttgcggatagcttttctttgctttctgttctaaaattatttcttagccattatttgatgatccaacttggtttgaggatctaataaaaattcaatttctttgaagcattcattggcttaatgatccaaaccttttgtcctcccactctctaagaaaataaagcaaaagcaaaactaaaagaaaagcaactagtaccgtgggttggtcaatggtaacccatgctaccaaataccaatcaaagcttaaatgacaatttagtaccttgggttggtcatgatttagagcggaacaaatataccatacatcaaacgaggatatacagagaaaattcatggaagtatggaatttagaggacaatttctcttctgggctacaacggaccggcccattcgctcatttacccagaggtatcgagtttgtggacttttgatgccgctagagactattggcatcggattgaaaaaaatgtttacttcgttgttaacaattttctgaatgcaatattttccttgtcatgaatatttaaacttgttattactttcttttagcttaggtgtggacaattatggagtttacttcaaacgaggaagaatcttaaacgctcggttcaggtctcgaaaacaaaccaactcacaatcctgacttaattgaggaaaacaatgcaaaaccacatgcatgcttccatatttctatcataatcttataagaaaaaacactactaagtgaaatgattctgtatatatataaccaatgccttttgttttgtgatattttatgtatatataactattgacttttgtcatctatggatagtgtctcgggctcttggcaaacatatttcaaagaaaagttaatgactgtaattaattaatctgaagctagaaacagaaccccgaggtaaaagaaaaagacagagcacatgaagtttagtacttttatatatttaatatatcattctttcttattgcttatctctaaagcaaaaacttccctaaaccctaagccaaaggactcagatcgatgcagaaccaagaaggcttgttttggatttgagagccaaatgcaaagaaaaaaactcttatgtctttgcaaatgccgatt

SEQ ID NO: 3 - DNA, 3960 bp, Arabidopsis thaliana
atgtctttgcaaatgccgattccaagaaaaaaagccttaactgtttctcatttttccattacattgtctctgtttttagctttcttcatctcctcgacctctgcatcaaccaatgaagtctcagctttgatttcttggcttcacagctcgaactcgccgccaccgtcagtcttctccggctggaatccttctgattctgacccatgtcaatggccttacattacttgttcctcctcagacaacaaactcgttacagagatcaatgtcgtctctgttcagttagctttacctttccctcctaacatttcttcgtttacttcacttcagaaactcgttatctccaacactaatctcaccggagctatatcttctgagatcggagattgttcggagcttatagttatcgatttaagctcaaatagtcttgttggtgaaataccttcgagtctagggaagctcaagaatcttcaagaactttgtttaaactccaatggcctcacaggaaagatcccaccggaactcggcgactgcgttagcctcaagaatcttgagattttcgataactacttatccgagaatctcccgttggagctcggaaagatctcgactcttgagagcataagagccggaggaaactcagagctttcagggaagatcccggaggagatcggaaactgtaggaatctcaaggtcttagggttagcagcaacgaaaatatccggttctttacctgtttcgttgggtcaactaagcaagctccagtctttatctgtctattctacaatgctctcaggtgagatccctaaagagcttggaaactgctctgaacttatcaatctgtttctatacgacaatgacttgtccggcacgcttccaaaagaactaggaaagcttcaaaacctagagaagatgcttctatggcagaataatctccacggacctatccctgaggagattggattcatgaaaagcttaaacgccattgatctctctatgaactatttctcaggaaccatccctaaatcgttcggtaacttgtcgaatctccaagagcttatgcttagcagcaacaacatcaccgggtcaataccttcgattctaagtaactgcacaaagcttgttcagttccagattgacgccaatcagatttcaggtttgattccaccagagattggattgcttaaggagctcaacatcttcttgggatggcagaataagctagaagggaatattccggacgagttagctggttgtcagaatcttcaagctcttgacttgtcacagaattatctcaccggatcattacctgcaggtttgtttcagcttcgtaatctgactaagctcttgcttatatctaatgccatctccggtgttatcccactggagatcgggaactgcacttcgcttgtcagattaaggcttgtcaataacagaatcaccggagaaatccctaaaggtattggatttcttcagaaccttagcttcctcgacttgtctgagaacaatctctccggtccagttcctttggagataagtaattgtagacaactccagatgcttaacttgagcaacaacactcttcaaggttatcttcctctgtctttatcatcgttaacaaagcttcaagtacttgatgtctcttccaatgacttgactggcaagatacctgatagtctaggtcatctcatttcactcaaccggctcattctaagtaagaattctttcaatggagaaatcccttcgtctctcggtcactgcacgaatcttcagcttcttgatctcagcagtaataacatctctggaactataccggaggagctttttgacatccaagatctagacattgcgttaaacttgagctggaattcattagatggctttatcccggaaaggatttccgcgcttaaccgattatccgtgctcgatatttcgcataacatgctttcaggcgacctttctgcgctgtccggtctagaaaacttggtttctctgaacatctctcataacagattctcaggttatcttccagatagtaaagtgtttagacagctgataggagcagagatggaaggaaacaatggactctgttccaaaggtttcaggtcttgctttgtaagtaacagttcacagttaactacacagcgtggtgtgcactcacatagactcaggatagccattggattgctaatcagcgtgacagcggttctagcggtattaggcgtgttggcggttatacgagctaaacaaatgattcgagatgataacgattcagagactggagaaaatctatggacatggcaattcacaccttttcagaaactcaacttcacagtcgaacatgtactcaagtgtttggtagaaggtaatgttatagggaaaggttgctccgggatagtgtacaaagctgaaatgcctaacagagaagtcatcgcggtgaaaaagctctggccagtgacagtacctaatctgaatgagaaaactaagtcatcaggagttcgagactcattctcagctgaagtaaaaacacttggatcgatcagacacaagaacattgtacgcttcttgggatgttgttggaacaagaacactagacttcttatgtatgattatatgtcaaatgggagtttgggaagtttgcttcacgaaaggagcggtgtatgtagcttaggatgggaagtgaggtacaagattatacttggtgcagctcagggtttggcttacttgcaccatgactgtgttcctcccattgttcatagagacatcaaggcaaataatattctgattggccctgattttgaaccttacattggagatttcggactcgctaagcttgttgatgatggcgactttgctcgttcttccaacaccattgctggttcctatggttacatagctccaggtacttcccaaatttctgttttgctgctaattagttaacttgatacatcataagttactagtagtcttagcctagtaatggtagaaattcaggtttatagtttggttttggtttttggcattttgaaaagaatagtattttggttcagttgattttcgatttggtttaaaattttagaaaaaaaatggtagaagaaataatttttgattaatattatatcatctactgtatacgatttaaaaaaaaaattgatttggtttggtctcgatattttgcttgtagcttggttcaatatttttactatatatatgattcatgactttcatactaatcaatatttgttcggtttcgttttgattagtctgaaagtgaatcatatagttaaaatattgaccaatctatcatatacttacacgctacaagattttcgatgtaattttggtgtaagcattttggtttttatatgttcatccttagtaactaacaacaaatatcaattgggaagcataacgttatatattcccataaggctataacactataattatgcagaatacggatactcaatgaagataacagagaaaagcgacgtgtacagctatggagtcgtagtgctcgaggtactaacgggtaagcaaccaatcgatccaacgataccagatggactccacatagtggactgggtcaagaaaatcagagacatacaagtaatcgaccaaggactacaagcaagaccagagtcagaggttgaagagatgatgcaaacgctaggagtcgcgcttttatgcattaatccaatacccgaagacaggcctacaatgaaagatgtggctgctatgcttagtgagatatgtcaagagagagaggaatcgatgaaagttgatggttgctcgggaagttgtaacaatggaagagaacgtggcaaagatgattcgacttcatcggttatgcaacaaacggctaagtatttgagaagtagtagcacgagtttctctgcgtcttctttgctttactcttcttcttcttctgctacttctaatgttagaccaaatcttaaataa

SEQ ID NO: 4 - protein, 1135 aa, Arabidopsis thaliana
MSLQMPIPRKKALTVSHFSITLSLFLAFFISSTSASTNEVSALISWLHSSNSPPPSVFSGWNPSDSDPCQWPYITCSSPDNKLVTEINVVSVQLALPFPPNISSFTSLQKLVISNTNLTGAISSEIGDCSELIVIDLSSNSLVGEIPSSLGKLKNLQELCLNSNGLTGKIPPELGDCVSLKNLEIFDNYLSENLPLELGKISTLESIRAGGNSELSGKIPEEIGNCRNLKVLGLAATKISGSLPVSLGQLSKLQSLFVYSTMLSGEIPKELGNCSELINLFLYDNDLSGTLPKELGKLQNLEKMLLWQNNLHGPIPEEIGFMKSLNAIDLSMNYFSGTIPKSFGNLSNLQELMLSSNNITGSIPSILSDCTKLVQFQIDANQISGLIPPEIGLLKELNIFLGWQNKLEGNIPDELAGCQNLQALDLSQNYLTGSLPAGLFQLRNLTKLLLISNAISGVIPLETGNCTSLVRLRLVNNRITGEIPKGIGFLQNLSFLDLSENNLSGPVPLEISNCRQLQMLNLSNNTLQGYLPLSLSSLTKLQVLDVSSNDLTGKIPDSLGHLISLNRLILSKNSFNGEIPSSLGHCTNLQLLDLSSNNISGTIPEELFDIQDLDIALNLSWNSLDGFIPERISALNRLSVLDISHNMLSGDLSALSGLENLVSLNISHNRFSGYLPDSKVFRQLIGAEMEGNNGLCSKGFRSCFVSNSSQLTTQRGVHSHRLRIAIGLLISVTAVLAVLGVLAVIRAKQMIRDDNDSETGENLWTWQFTPFQKLNFTVEHVLKCLVEGNVIGKGCSGIVYKAEMPNREVIAVKKLWPVTVPNLNEKTKSSGVRDSFSAEVKTLGSIRHKNIVRFLGCCWNKNTRLLMYDYMSNGSLGSLLHERSGVCSLGWEVRYKIILGAAQGLAYLHHDCVPPIVHRDIKANNILIGPDFEPYIGDFGLAKLVDDGDFARSSNTIAGSYGYIAPEYGYSMKITEKSDVYSYGVVVLEVLTGKQPIDPTIPDGLHIVDWVKKIRDIQVIDQGLQARPESEVEEMMQTLGVALLCINPIPEDRPTMKDVAAMLSEICQEREESMKVDGCSGSCNNGRERGKDDSTSSVMQQTAKYLRSSSTSFSASSLLYSSSSSATSNVRPNLK

SEQ ID NO: 5 - protein, 25 aa, Arabidopsis thaliana
RYKIILGAAQGLAYLHHDCVPPIVH

SEQ ID NO: 6 - DNA, 23 bp, Arabidopsis thaliana
  misc_feature (3)..(3): y = c or t
aayaayttmasyggtkmdwthcc

SEQ ID NO: 7 - DNA, 20 bp, Arabidopsis thaliana
  misc_feature (3)..(3): r = g or a
carythamcggwgaaathcc

SEQ ID NO: 8 - DNA, 20 bp, Arabidopsis thaliana
  misc_feature (1)..(1): m = a or c
mayytsavyggahctathcc

SEQ ID NO: 9 - DNA, 22 bp, Arabidopsis thaliana
  misc_feature (2)..(2): h = a or c or t
thccwttwgscatgtantcata

SEQ ID NO: 10 - DNA, 19 bp, Arabidopsis thaliana
  misc_feature (2)..(2): y = c or t
aytcdggdgchahgtadcc

SEQ ID NO: 11 - DNA, 21 bp, Arabidopsis thaliana
  misc_feature (1)..(1): m = a or c
mayhccraarctrtavacatc

SEQ ID NO: 12 - DNA, 19 bp, Arabidopsis thaliana
cgatcagacacaagaacat

SEQ ID NO: 13 - DNA, 19 bp, Arabidopsis thaliana
agcaatggtgttggaagaa

SEQ ID NO: 14 - DNA, 19 bp, Arabidopsis thaliana
  misc_feature (11)..(11): y = c or t
tgcagatcttygtgaagac

SEQ ID NO: 15 - DNA, 18 bp, Arabidopsis thaliana
gactccttctggatgttg

SEQ ID NO: 16 - DNA, 18 bp, Arabidopsis thaliana
tgtaaaacgacggccagt

SEQ ID NO: 17 - DNA, 34 bp, Arabidopsis thaliana
cacacaggatccaatcggcatttgcaaagacata

SEQ ID NO: 18 - DNA, 842 bp, Arabidopsis thaliana
aagcttaaatgacaatttagtaccttgggttggtcatgatttagagcggaacaaatataccatacatcaaacgaggatatacagagaaaattcatggaagtatggaatttagaggacaatttctcttctgggctacaacggaccggcccattcgctcatttacccagaggtatcgagtttgtggacttttgatgccgctagagactattggcatcggattgaaaaaaatgtttacttcgttgttaacaattttctgaatgcaatattttccttgtcatgaatatttaaacttgttattactttcttttagcttaggtgtggacaattatggagtttacttcaaacgaggaagaatcttaaacgctcggttcaggtctcgaaaacaaaccaactcacaatcctgacttaattgaggaaaacaatgcaaaaccacatgcatgcttccatatttctatcataatcttataagaaaaaacactactaagtgaaatgattctgtatatatataaccaatgccttttgttttgtgatattttatgtatatataactattgacttttgtcatctatggatagtgtctcgggctcttggcaaacatatttcaaagaaaagttaatgactgtaattaattaatctgaagctagaaacagaaccccgaggtaaaagaaaaagacagagcacatgaagtttagtacttttatatatttaatatatcattctttcttattgcttatctctaaagcaaaaacttccctaaaccctaagccaaaggactcagatcgatgcagaaccaagaaggcttgttttggatttgagagccaaatgcaaagaaaaaaactctt

SEQ ID NO: 19 - protein, 1130 aa, Arabidopsis thaliana
MPIPRKKALTVSHFSITLSLFLAFFISSTSASTNEVSALISWLHSSNSPPPSVFSGWNPSDSDPCQWPYITCSSPDNKLVTEINVVSVQLALPFPPNISSFTSLQKLVISNTNLTGAISSEIGDCSELIVIDLSSNSLVGEIPSSLGKLKNLQELCLNSNGLTGKIPPELGDCVSLKNLEIFDNYLSENLPLELGKISTLESIRAGGNSELSGKIPEEIGNCRNLKVLGLAATKISGSLPVSLGQLSKLQSLFVYSTMLSGEIPKELGNCSELINLFLYDNDLSGTLPKELGKLQNLEKMLLWQNNLHGPIPEEIGFMKSLNAIDLSMNYFSGTIPKSFGNLSNLQELMLSSNNITGSIPSILSDCTKLVQFQIDANQISGLIPPEIGLLKELNIFGWQNKLEGNIPDELAGCQNLQALDLSQNYLTGSLPAGLFQLRNLTKLLLISNAISGVIPLETGNCTSLVRLRLVNNRITGEIPKGIGFLQNLSFLDLSENNLSGPVPLEISNCRQLQMLNLSNNTLQGYLPLSLSSLTKLQVLDVSSNDLTGKIPDSLGHLISLNRLILSKNSFNGEIPSSLGHCTNLQLLDLSSNNISGTIPEELFDIQDLDIALNLSWNSLDGFIPERISALNRLSVLDISHNMLSGDLSALSGLENLVSLNISHNRFSGYLPDSKVFRQLIGAEMEGNNGLCSKGFRSCFVSNSSQLTTQRGVHSHRLRIAIGLLISVTAVLAVLGVLAVIRAKQMIRDDNDSETGENLWTWQFTPFQKLNFTVEHVLKCLVEGNVIGKGCSGIVYKAEMPNREVIAVKKLWPVTVPNLNEKTKSSGVRDSFSAEVKTLGSIRHKNIVRFLGCCWNKNTRLLMYDYMSNGSLGSLLHERSGVCSLGWEVRYKIILGAAQGLAYLHHDCVPPIVHRDIKANNILIGPDFEPYIGDFGLAKLVDDGDFARSSNTIAGSYGYIAPEYGYSMKITEKSDVYSYGVVVLEVLTGKQPIDPTIPDGLHIVDWVKKIRDIQVIDQGLQARPESEVEEMMQTLGVALLCINPIPEDRPTMKDVAAMLSEICQEREESMKVDGCSGSCNNGRERGKDDSTSSVMQQTAKYLRSSSTSFSASSLLYSSSSSATSNVRPNLK